(19) United States
(12) Patent Application Publication
(10) Pub. No.: US 2024/0060079 A1
(43) Pub. Date: Feb. 22, 2024

(54) METHODS FOR PRODUCING VANILLA PLANTS WITH IMPROVED FLAVOR AND AGRONOMIC PRODUCTION

(71) Applicants: Elo Life Systems, Durham, NC (US); University of Florida Research Foundation Incorporated, Gainesville, FL (US)

(72) Inventors: Fayaz Khazi, Chapel Hill, NC (US); Tengfang Huang, Cary, NC (US); Haibao Tang, Mountain View, CA (US); Tomas Hasing, Durham, NC (US); Alan Chambers, Homestead, FL (US)

(21) Appl. No.: 18/033,170

(22) PCT Filed: Oct. 25, 2021
(86) PCT No.: PCT/US2021/056474

§ 371 Date: Apr. 21, 2023

Related U.S. Application Data
(60) Provisional application No. 63/104,891, filed on Oct. 23, 2020.

(51) Int. Cl. C12N 15/82; C07K 14/415; C12N 9/50; C12N 9/88

(52) U.S. Cl. CPC C12N 15/8243; C07K 14/415; C12N 9/63; C12N 9/88; C12N 15/8266; C12N 15/8282; C12Y 403/01024

(57) ABSTRACT

Provided herein are methods for improving various traits in plants, such as in Vanilla sp. plants, by genetically-modifying the genome of plants, introducing heterologous sequences into the plants, or breeding plants with selected alleles. Such traits that can be improved by the methods and compositions of the present invention include an increase in levels of vanillin or one or more precursors thereof, reducing dehiscence, reducing the size of a rostellum or eliminating its presence, and increasing fungal resistance. Also disclosed herein are genetically-modified plants, extract from such plants or plant parts thereof, and methods of producing such plants or progeny thereof.

### CROSS-REFERENCE TO RELATED APPLICATIONS

This application claims priority to U.S. Provisional Application No. 63/104,891, filed Oct. 23, 2020, which is incorporated by reference herein in its entirety.

### STATEMENT REGARDING THE SEQUENCE LISTING

The Sequence Listing associated with this application is provided in ASCII format in lieu of a paper copy, and is hereby incorporated by reference into the specification. The ASCII copy named P89339_1140WO_0123_9_SL.txt is 125,386 bytes in size, was created on Oct. 25, 2021, and is being submitted electronically via EFS-Web.

### FIELD OF THE INVENTION

The invention relates to the fields of genetics, molecular biology, and botany. In particular, the invention relates to the production of plants, particularly Vanilla sp. plants, through genetic modification or breeding of plants with selected alleles.

### BACKGROUND OF THE INVENTION

Vanilla, the world's most popular flavor, is sourced from the tropical orchid species Vanilla planifolia. Vanilla was clonally propagated, cultivated, and globally distributed from wild plants as part of the early spice trade. Today, the industry struggles to meet global demand and is inhibited by inefficient and unsustainable practices due to the lack of genomic and technical resources that enable germplasm improvement.

High consumer demand for natural ingredients has led to increased use of natural vanilla extract in food, beverage, pharmaceutical, and cosmetic industries around the world. The market for vanilla products obtained from the two commercial species (V. planifolia and V. x tahitensis) is projected to exceed $4.3B by 2025 (Acumen. Vanilla Beans and Extract Market Worth US$4.3 Bn by 2025. Acumen Research and Consulting, Los Angeles, 2019) with the United States and Europe being the two largest importers of vanilla beans. Vanilla plants were taken from North and Central America by Spanish conquistadores as part of the early spice trade in the early 16th century and distributed globally (Childers. (1948) Vanilla culture in Puerto Rico, US Department of Agriculture 28). Vanilla thrived in new geographies that now include today's major vanilla growing areas of Madagascar, Indonesia, Uganda, India, and others (Medina et al., (2009) Vanilla: Post-harvest operations. Food and Agriculture Organization of the United Nations). Today, vanilla is still primarily propagated asexually by cuttings, and vines in commercial production are identical to the original wild and undomesticated clones. As a consequence, there is limited genetic diversity within and across growing regions due to the mass propagation of a few foundational clones and the lack of sexual recombination. Conversely, minor vanilla cultivation in North and Central America, which are near the center of diversity for V. planifolia, often rely on uncharacterized wild populations with visible morphological variation. Understanding and leveraging vanilla genetic diversity and the mechanisms responsible for resilience and quality are key to strengthening and stabilizing the vanilla supply chain.

Vanilla seed capsules (commonly called beans or pods) are collected from remote growing locations and laboriously transported to centralized curing facilities. Curing involves multiple steps of defined heat treatments while gradually reducing bean moisture content which results in development of the full vanilla aroma and stabilizes the beans for shipping. The long, cured, aromatic, and unsplit beans are highly desired in the marketplace. This artisanal approach to vanilla production introduces multiple threats impacting the productivity and sustainability of the vanilla supply chain. The majority of the limitations impacting the supply chain are directly influenced by genetics and could be improved through plant breeding or genome editing (Chambers, Advances in Plant Breeding Strategies: Industrial and Food Crops, Ch. 18 (Springer, 2019); Chambers et al., (2019) Vanilla Cultivation in Southern Florida, EDIS; Sasikumar (2010) Vanilla breeding—a review. Agric. Rev. 31; Lepers-Andrzejewski et al. (2012) Crop Sci 52:795-806). For example, vanillin, a key phenolic flavor compound, is often measured as a proxy representing overall extract quality, though vanillin is only one of over one hundred aroma volatiles in vanilla extract. Higher vanillin content is desirable, but the natural biosynthetic pathway of vanillin has yet to be fully elucidated (Yang et al. (2017) Phytochem. 139:33-46). Multiple genes likely to impact vanillin abundance and extract quality are yet to be identified and characterized.

Vanilla beans achieve higher quality the longer they mature on the vine, however, they are also more likely to split (bean dehiscence) as a part of their natural seed dispersal mechanism at maturity. Since split beans are undesirable in commercial operations for many species, one of the first steps in crop domestication is selection against bean dehiscence or seed shattering. The coordinated formation of an abscission layer in vanilla beans shares common processes with diverse crops from cereals to legumes and many genes have been found to interrupt this process and reduce yield loss (Dong and Wang (2015) Front. Plant Sci. 6, doi:10.3389/fpls.201500476). In contrast to V. planifolia, V. x tahitensis beans are indehiscent and ripen on the vine without splitting (Lapeyre-Montes et al. in Vanilla Medicinal and Aromatic Plants—Industrial Profiles (ed. Eric Odoux and Michel Grisoni) Ch. 10 (CRC Press, 2010)). Understanding the genes involved in regulating bean dehiscence can allow for disruption of this pathway via genome editing, for example, to produce genetically-modified unsplit vanilla beans.

Other traits impact vanilla production. For example, due to the absence of natural pollinators in major vanilla growing regions, nearly all vanilla flowers must be hand pollinated for bean development to circumvent the flap-like organ (the rostellum) that physically separates the male and female parts of the flower (FIG. 1) (Soto-Arenas et al. (2003) Genera Orchidacearum 3:321-334). This introduces a significant human and economic burden into the food system. Understanding the genetic basis for developmental differences between V. planifolia (commercial quality with a rostellum) and V. mexicana (non-aromatic without a rostellum), for example, could identify specific gene targets for genome modification or breeding efforts focused on eliminating the rostellum organ and improvement of production economics (Chambers, Advances in Plant Breeding Strategies: Industrial and Food Crops, Ch. 18 (Springer, 2019); Gigant et al. in Microsatellite Markers (InTech, 2016)). Also, the vast monoculture of clonally-related vanilla plants portends catastrophic disease epidemics such as those currently threatening the citrus and banana industries (National Academies of Sciences, E. & Medicine. A review of the citrus greening research and development efforts supported by the Citrus Research and Development Foundation: Fighting a ravaging disease. (National Academies Press, 2018); Ploetz (2015) Phytopathol. 105:1512-1521). While all commercial vanilla is susceptible to a fungal pathogen (Fusarium oxysporum f. sp. vanillae), related species such as V. pompona are resistant to the pathogen and could provide a genetic route to creating disease resistant V. planifolia (Childers. (1948) Vanilla culture in Puerto Rico, US Department of Agriculture 28; Delassus (1963) L'Agronomie Tropicale. Série 2, Agronomie Générale. Etudes Techniques 18:245-246) via classical breeding or new breeding technologies such as genome editing.

### SUMMARY OF THE INVENTION

Provided herein are methods for improving various traits in plants, such as in Vanilla sp. plants, by breeding or genetically-modifying the genome of plants or introducing heterologous sequences into the plants. Such traits that can be improved by the methods and compositions of the present invention include an increase in levels of vanillin or one or more precursors thereof, reducing dehiscence (i.e., seed shattering), reducing the size of a rostellum or eliminating its presence, and increasing fungal resistance. Also disclosed herein are genetically-modified plant cells, plant parts, or plants (such as a Vanilla sp. plant), extract from such plants, or plant parts (such as beans) from such plants, methods of producing such plants or progeny of such plants or a population of such plants or progeny thereof.

Accordingly, in one aspect, the invention provides a method for increasing the expression of a phenylalanine ammonia lyase (PAL) in a Vanilla sp. plant cell by introducing into said plant cell a nucleic acid molecule comprising a nucleotide sequence encoding a PAL or by genetically-modifying the endogenous promoter of a gene encoding PAL.

In some embodiments of the method, the nucleic acid molecule further comprises a heterologous promoter operably linked to said nucleotide sequence encoding PAL. In some of these embodiments, the promoter is a pod, mesocarp, placenta, or seed-specific promoter.

In certain embodiments of the method, genetically-modifying the endogenous promoter comprises replacing said endogenous promoter with a heterologous promoter.

In certain embodiments, the method comprises introducing at least one copy of a gene encoding a phenylalanine ammonia lyase (PAL) polypeptide into the genome of a Vanilla sp. plant cell by genetically-modifying the genome of the Vanilla sp. plant cell to comprise at least two copies of the gene to generate a genetically-modified Vanilla sp. plant cell.

In some embodiments of the method, two copies of the gene are within the endogenous genomic locus. In particular embodiments, the Vanilla sp. plant cell has a diploid genome.

The PAL polypeptide that is encoded by the gene can comprise an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1. In some of these embodiments, the PAL polypeptide retains one or more of the amino acid residues that are present in SEQ ID NO: 1, but not SEQ ID NO: 3, 5, and 7, as shown in the alignment provided in FIG. 16, respectively. In certain embodiments, the gene encoding PAL has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 2.

In certain embodiments of the method, the genetically-modified Vanilla sp. plant cell has increased levels of PAL compared to a non-genetically modified Vanilla sp. plant cell. In particular embodiments, the genetically-modified Vanilla sp. plant cell produces increased levels of cinnamic acid compared to a non-genetically-modified Vanilla sp. plant cell.

In particular embodiments of the method, the Vanilla sp. plant cell is within a seed or a seed capsule. In certain embodiments, the Vanilla sp. plant cell is a Vanilla planifolia, Vanilla x tahitensis, or Vanilla pompona plant cell. A Vanilla sp. plant or plant part comprising the genetically-modified Vanilla sp. plant cell can have increased levels of vanillin or one or more precursors thereof compared to a Vanilla sp. plant or plant part not comprising the genetically-modified Vanilla sp. plant cell. In some of these embodiments, the Vanilla sp. plant part comprises a bean.

In another aspect, the invention provides a method for producing a Vanilla sp. plant having increased expression of a phenylalanine ammonia lyase (PAL) in a Vanilla sp. plant cell or plant part by introducing into said plant cell a nucleic acid molecule comprising a nucleotide sequence encoding a PAL or by genetically-modifying the endogenous promoter of a gene encoding PAL, which generates a genetically-modified Vanilla sp. plant cell or plant part, followed by growing a Vanilla sp. plant from the genetically-modified Vanilla sp. plant cell or plant part. The produced Vanilla sp. plant has increased expression of PAL as compared to a suitable control plant, such as one that has not been genetically modified according to the method.

In some embodiments of the method, the nucleic acid molecule further comprises a heterologous promoter operably linked to said nucleotide sequence encoding PAL. In some of these embodiments, the promoter is a pod, mesocarp, placenta, or seed-specific promoter.

In certain embodiments of the method, genetically-modifying the endogenous promoter comprises replacing said endogenous promoter with a heterologous promoter.

In some embodiments, the method comprises producing a Vanilla sp. plant having at least two copies of a gene encoding a PAL polypeptide by genetically-modifying the genome of a Vanilla sp. plant cell or plant part to comprise at least two copies of the gene, which generates a genetically-modified Vanilla sp. plant cell or plant part, followed by growing a Vanilla sp. plant from the genetically-modified Vanilla sp. plant cell or plant part. The produced Vanilla sp. plant has at least two copies of the gene.

In some embodiments of the method, the Vanilla sp. plant has increased levels of PAL compared to a control plant. In certain embodiments, the genetically-modified Vanilla sp. plant cell or plant part produces increased levels of cinnamic acid compared to a non-genetically-modified Vanilla sp. plant cell or plant part.

In particular embodiments of the method, two copies of the gene are within the endogenous genomic locus. In some embodiments, the Vanilla sp. plant cell has a diploid genome.

The PAL polypeptide that is encoded by the gene can comprise an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1. In some of these embodiments, the PAL polypeptide retains one or more of the amino acid residues that are present in SEQ ID NO: 1, but not SEQ ID NO: 3, 5, and 7, as shown in the alignment provided in FIG. 16. In certain embodiments, the gene encoding PAL has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 2.

In particular embodiments of the method, the Vanilla sp. plant is a Vanilla planifolia, Vanilla x tahitensis, or Vanilla pompona plant. A bean of the Vanilla sp. plant can have increased levels of vanillin or one or more precursors thereof compared to a control plant.

In still another aspect, the invention provides a genetically-modified Vanilla sp. plant cell having increased expression of a PAL as compared to a suitable control plant, such as one that has not been genetically modified.

In some embodiments, the genetically-modified Vanilla sp. plant cell is one in which a nucleic acid molecule comprising a nucleotide sequence encoding a PAL polypeptide has been stably integrated into the genome of the Vanilla sp. plant cell or the endogenous promoter of a gene encoding PAL of the Vanilla sp. plant cell has been genetically-modified to increase expression.

In some embodiments, the nucleic acid molecule further comprises a heterologous promoter operably linked to said nucleotide sequence encoding PAL. In some of these embodiments, the promoter is a pod, mesocarp, placenta, or seed-specific promoter.

In certain embodiments, the endogenous promoter of the gene encoding PAL has been replaced by a heterologous promoter.

In certain embodiments, the genetically-modified Vanilla sp. plant cell has at least two copies of a gene encoding a PAL, wherein the genome of the genetically-modified Vanilla sp. plant cell comprises a genetic modification such that the genetically-modified Vanilla sp. plant cell comprises the at least two copies of the gene.

In particular embodiments of the composition, two copies of the gene are within the endogenous genomic locus. In some embodiments, the genetically-modified Vanilla sp. plant cell has a diploid genome.

The PAL polypeptide that is encoded by the gene can comprise an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1. In some of these embodiments, the PAL polypeptide retains one or more of the amino acid residues that are present in SEQ ID NO: 1, but not SEQ ID NO: 3, 5, and 7, as shown in the alignment provided in FIG. 16. In certain embodiments, the gene encoding PAL at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 2.

In some embodiments of the composition, the genetically-modified Vanilla sp. plant cell has increased levels of PAL compared to a non-genetically-modified Vanilla sp. plant cell. In certain embodiments, the genetically-modified Vanilla sp. plant cell produces increased levels of cinnamic acid compared to a non-genetically-modified Vanilla sp. plant cell.

In particular embodiments of the composition, the genetically-modified Vanilla sp. plant cell is within a seed or a seed capsule. In certain embodiments, the genetically-modified Vanilla sp. plant cell is a Vanilla planifolia, Vanilla x tahitensis, or Vanilla pompona genetically-modified plant cell.

A Vanilla sp. plant or plant part comprising a genetically-modified Vanilla sp. plant cell is provided. The Vanilla sp. plant or plant part can have increased levels of vanillin or one or more precursors thereof compared to a control plant or plant part. In some of these embodiments, the Vanilla sp. plant part comprises a bean. An extract from the Vanilla sp. plant or plant part comprising the genetically-modified Vanilla sp. plant cell is also provided.

In yet another aspect, the invention provides a method for increasing the expression of a cysteine protease-like protein (CPLP) in a Vanilla sp. plant cell by introducing into said plant cell a nucleic acid molecule comprising a nucleotide sequence encoding a CPLP or by genetically-modifying the endogenous promoter of a gene encoding CPLP.

In some embodiments of the method, the nucleic acid molecule further comprises a heterologous promoter operably linked to said nucleotide sequence encoding CPLP. In some of these embodiments, the promoter is a pod, mesocarp, placenta, or seed-specific promoter.

In certain embodiments of the method, genetically-modifying the endogenous promoter comprises replacing said endogenous promoter with a heterologous promoter.

In certain embodiments, the method comprises introducing at least one copy of a gene encoding a cysteine protease-like protein (CPLP) into the genome of a Vanilla sp. plant cell by genetically-modifying the genome of the Vanilla sp. plant cell to comprise at least two copies of the gene and generating a genetically-modified Vanilla sp. plant cell.

In some embodiments of the method, two copies of the gene are within the endogenous genomic locus. In particular embodiments, the Vanilla sp. plant cell has a diploid genome.

In certain embodiments, the gene encodes a CPLP transcript comprising exons 1-3. The CPLP polypeptide that is encoded by the gene can comprise an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9 or 11. In some of these embodiments, the CPLP polypeptide retains amino acid residues 1-144 and/or a serine at a position corresponding to 151 of SEQ ID NO: 9 or 11. In certain embodiments, the gene encoding CPLP has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10 or 12.

In certain embodiments of the method, the genetically-modified Vanilla sp. plant cell has increased levels of CPLP compared to a non-genetically modified Vanilla sp. plant cell. In particular embodiments, the genetically-modified Vanilla sp. plant cell produces increased levels of at least one of 4-hydroxybenzaldehyde and vanillin compared to a non-genetically-modified Vanilla sp. plant cell.

In particular embodiments of the method, the Vanilla sp. plant cell is within a seed or a seed capsule. In certain embodiments, the Vanilla sp. plant cell is a Vanilla planifolia, Vanilla x tahitensis, or Vanilla pompona plant cell. A Vanilla sp. plant or plant part comprising the genetically-modified Vanilla sp. plant cell can have increased levels of vanillin or one or more precursors thereof compared to a Vanilla sp. plant or plant part not comprising the genetically-modified Vanilla sp. plant cell. In some of these embodiments, the Vanilla sp. plant part comprises a bean.

In another aspect, the invention provides a method for producing a Vanilla sp. plant having increased expression of a CPLP in a Vanilla sp. plant cell or plant part by introducing into said plant cell a nucleic acid molecule comprising a nucleotide sequence encoding a CPLP or by genetically-modifying the endogenous promoter of a gene encoding CPLP, which generates a genetically-modified Vanilla sp. plant cell or plant part, followed by growing a Vanilla sp. plant from the genetically-modified Vanilla sp. plant cell or plant part. The produced Vanilla sp. plant has increased expression of CPLP as compared to a suitable control plant, such as one that has not been genetically modified according to the method.

In some embodiments of the method, the nucleic acid molecule further comprises a heterologous promoter operably linked to said nucleotide sequence encoding CPLP. In some of these embodiments, the promoter is a pod, mesocarp, placenta, or seed-specific promoter.

In certain embodiments of the method, genetically-modifying the endogenous promoter comprises replacing said endogenous promoter with a heterologous promoter.

In some embodiments, the method comprises producing a Vanilla sp. plant having at least two copies of a gene encoding a CPLP polypeptide by genetically-modifying the genome of a Vanilla sp. plant cell or plant part to comprise at least two copies of the gene, which generates a genetically-modified Vanilla sp. plant cell or plant part, followed by growing a Vanilla sp. plant from the genetically-modified Vanilla sp. plant cell or plant part. The produced Vanilla sp. plant has at least two copies of the gene.

In some embodiments of the method, the Vanilla sp. plant has increased levels of CPLP compared to a control plant. In certain embodiments, the genetically-modified Vanilla sp. plant cell or plant part produces increased levels of at least one of 4-hydroxybenzaldehyde and vanillin compared to a non-genetically-modified Vanilla sp. plant cell or plant part.

In particular embodiments of the method, two copies of the gene are within the endogenous genomic locus. In some embodiments, the plant cell has a diploid genome.

In certain embodiments, the gene encodes a CPLP transcript comprising exons 1-3. The CPLP polypeptide that is encoded by the gene can comprise an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9 or 11. In some of these embodiments, the CPLP polypeptide retains amino acid residues 1-144 and/or a serine at a position corresponding to 151 of SEQ ID NO: 9 or 11. In certain embodiments, the gene encoding CPLP has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10 or 12.

In particular embodiments of the method, the plant is a Vanilla planifolia, Vanilla x tahitensis, or Vanilla pompona plant. A bean of the Vanilla sp. plant can have increased levels of vanillin or one or more precursors thereof compared to a control plant.

In still another aspect, the invention provides a genetically-modified Vanilla sp. plant cell having increased expression of a CPLP polypeptide as compared to a suitable control plant, such as one that has not been genetically modified.

In some embodiments, the genetically-modified Vanilla sp. plant cell is one in which a nucleic acid molecule comprising a nucleotide sequence encoding a CPLP polypeptide has been stably integrated into the genome of the Vanilla sp. plant cell or the endogenous promoter of a gene encoding CPLP of the Vanilla sp. plant cell has been genetically-modified to increase expression.

In some embodiments, the nucleic acid molecule further comprises a heterologous promoter operably linked to said nucleotide sequence encoding CPLP. In some of these embodiments, the promoter is a pod, mesocarp, placenta, or seed-specific promoter.

In certain embodiments, the endogenous promoter of the gene encoding CPLP has been replaced by a heterologous promoter.

In certain embodiments, the genetically-modified Vanilla sp. plant cell has at least two copies of a gene encoding a CPLP, wherein the genome of the genetically-modified Vanilla sp. plant cell comprises a genetic modification such that the genetically-modified Vanilla sp. plant cell comprises the at least two copies of the gene.

In particular embodiments of the composition, two copies of the gene are within the endogenous genomic locus. In some embodiments, the genetically-modified plant cell has a diploid genome.

In certain embodiments, the gene encodes a CPLP transcript comprising exons 1-3.

The CPLP polypeptide that is encoded by the gene can comprise an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9 or 11. In some of these embodiments, the CPLP polypeptide retains amino acid residues 1-144 and/or a serine at a position corresponding to 151 of SEQ ID NO: 9 or 11. In certain embodiments, the gene encoding CPLP has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10 or 12.

In some embodiments of the composition, the genetically-modified Vanilla sp. plant cell has increased levels of CPLP compared to a non-genetically-modified plant cell. In certain embodiments, the genetically-modified plant cell produces increased levels of at least one of 4-hydroxybenzaldehyde and vanillin compared to a non-genetically-modified Vanilla sp. plant cell.

In particular embodiments of the composition, the genetically-modified Vanilla sp. plant cell is within a seed or a seed capsule. In certain embodiments, the genetically-modified Vanilla sp. plant cell is a genetically modified Vanilla planifolia, Vanilla x tahitensis, or Vanilla pompona genetically-modified plant cell.

A Vanilla sp. plant or plant part comprising the genetically-modified Vanilla sp. plant cell is provided. The Vanilla sp. plant or plant part can have increased levels of vanillin or one or more precursors thereof compared to a control Vanilla sp. plant or plant part. In some of these embodiments, the Vanilla sp. plant part comprises a bean. An extract from the Vanilla sp. plant or plant part comprising the genetically-modified Vanilla sp. plant cell is also provided.

In another aspect, the invention provides a method for introducing at least one indehiscence-associated mutation into at least one dehiscent gene or reducing the expression of at least one dehiscent gene in a Vanilla sp. plant cell by genetically-modifying the genome of the Vanilla sp. plant cell to introduce the at least one indehiscence-associated mutation into the at least one dehiscent gene or to reduce the expression of the at least one dehiscent gene to generate a genetically-modified Vanilla sp. plant cell, wherein the dehiscent gene encodes a Shatterproof, Indehiscent, Replumless, Adpg1, or Sh1 protein.

In particular embodiments, at least one copy of at least one dehiscent gene is disrupted or knocked out. In some of these embodiments, all copies of at least one dehiscent gene is disrupted or knocked out.

In other embodiments, genetically-modifying the Vanilla sp. genome comprises mutating at least one dehiscent gene such that the activity of the encoded protein is reduced. In some of these embodiments, mutating the gene comprises introducing at least one missense mutation. In other embodiments, mutating the gene comprises introducing at least one nonsense mutation such that the dehiscent gene encodes a truncated protein.

In specific embodiments of the method, genetically-modifying the genome of the Vanilla sp. plant cell comprises introducing at least one indehiscence-associated mutation into the dehiscent gene encoding a Shatterproof protein. In some of these embodiments, the indehiscence-associated mutation is selected from one that results in: a) a leucine at a position corresponding to 149 of SEQ ID NO: 15; and b) a tyrosine at a position corresponding to 165 of SEQ ID NO: 15. In particular embodiments, genetically-modifying the genome of the Vanilla sp. plant cell comprises introducing each of the indehiscence-associated mutations of a) and b).

In other specific embodiments of the method, genetically-modifying the genome of the Vanilla sp. plant cell comprises introducing at least one indehiscence-associated mutation into the dehiscent gene encoding an Indehiscent protein. In some of these embodiments, the indehiscence-associated mutation is selected from one that results in: a) a serine inserted in between positions corresponding to 45 and 46 of SEQ ID NO: 17; and b) a proline at a position corresponding to 35 of SEQ ID NO: 17. In particular embodiments, genetically-modifying the genome of the Vanilla sp. plant cell comprises introducing each of the indehiscence-associated mutations of a) and b).

In still other specific embodiments of the method, genetically-modifying the genome of the Vanilla sp. plant cell comprises introducing at least one indehiscence-associated mutation into the dehiscent gene encoding a Replumless protein. In some of these embodiments, the indehiscence-associated mutation results in a glycine at a position corresponding to 10 of SEQ ID NO: 19.

In yet other specific embodiments of the method, genetically-modifying the genome of the Vanilla sp. plant cell comprises introducing at least one indehiscence-associated mutation into the dehiscent gene encoding an Adpg1 protein. In some of these embodiments, the indehiscence-associated mutation is selected from one that results in: a) a tryptophan at a position corresponding to 29 of SEQ ID NO: 21; b) a serine at a position corresponding to 15 of SEQ ID NO: 21; and c) an aspartic acid at a position corresponding to 12 of SEQ ID NO: 21. In particular embodiments, genetically-modifying the genome of the Vanilla sp. plant cell comprises introducing each of the indehiscence-associated mutations of a), b), and c).

In other specific embodiments of the method, genetically-modifying the genome of the Vanilla sp. plant cell comprises introducing at least one indehiscence-associated mutation into the dehiscent gene encoding a Sh1 protein. In some of these embodiments, the indehiscence-associated mutation results in a threonine at a position corresponding to 113 of SEQ ID NO: 23.

In certain embodiments, the Vanilla sp. plant cell is within a seed or a seed capsule. In some embodiments, the Vanilla sp. plant cell is Vanilla planifolia, Vanilla pompona, or Vanilla odorata. In particular embodiments of the method, a Vanilla sp. plant or bean comprising the genetically-modified Vanilla sp. plant cell has reduced dehiscence compared to a Vanilla sp. plant or bean not comprising the genetically-modified plant cell.

In another aspect, the invention provides a method for producing a Vanilla sp. plant having at least one indehiscence-associated mutation in at least one dehiscent gene or reduced expression of at least one dehiscent gene by: a) genetically-modifying the genome of a Vanilla sp. plant cell or plant part to introduce the at least one indehiscence-associated mutation into the at least one dehiscent gene or to reduce the expression of the at least one dehiscent gene to generate a genetically-modified plant cell or plant part, wherein the dehiscent gene encodes a Shatterproof, Indehiscent, Replumless, Adpg1, or Sh1 protein; and b) growing a plant from the genetically-modified plant cell or plant part, wherein the plant has the at least one indehiscence-associated mutation in the at least one dehiscent gene or reduced expression of the at least one dehiscent gene compared to a control plant.

In some embodiments of the method, the Vanilla sp. plant has reduced dehiscence compared to a control Vanilla sp. plant. In particular embodiments, at least one copy of at least one dehiscent gene is disrupted or knocked out. In some of these embodiments, all copies of at least one dehiscent gene is disrupted or knocked out.

In other embodiments, genetically-modifying the Vanilla sp. genome comprises mutating at least one dehiscent gene such that the activity of the encoded protein is reduced. In some of these embodiments, mutating the gene comprises introducing at least one missense mutation. In other embodiments, mutating the gene comprises introducing at least one nonsense mutation such that the dehiscent gene encodes a truncated protein.

In specific embodiments of the method, genetically-modifying the Vanilla sp. genome comprises introducing at least one indehiscence-associated mutation into the dehiscent gene encoding a Shatterproof protein. In some of these embodiments, the indehiscence-associated mutation is selected from one that results in: a) a leucine at a position corresponding to 149 of SEQ ID NO: 15; and b) a tyrosine at a position corresponding to 165 of SEQ ID NO: 15. In particular embodiments, genetically-modifying the Vanilla sp. genome comprises introducing each of the indehiscence-associated mutations of a) and b).

In other specific embodiments of the method, genetically-modifying the Vanilla sp. genome comprises introducing at least one indehiscence-associated mutation into the dehiscent gene encoding an Indehiscent protein. In some of these embodiments, the indehiscence-associated mutation is selected from one that results in: a) a serine inserted in between positions corresponding to 45 and 46 of SEQ ID NO: 17; and b) a proline at a position corresponding to 35 of SEQ ID NO: 17. In particular embodiments, genetically-modifying the Vanilla sp. genome comprises introducing each of the indehiscence-associated mutations of a) and b).

In still other specific embodiments of the method, genetically-modifying the Vanilla sp. genome comprises introducing at least one indehiscence-associated mutation into the dehiscent gene encoding a Replumless protein. In some of these embodiments, the indehiscence-associated mutation results in a glycine at a position corresponding to 10 of SEQ ID NO: 19.

In yet other specific embodiments of the method, genetically-modifying the Vanilla sp. genome comprises introducing at least one indehiscence-associated mutation into the dehiscent gene encoding an Adpg1 protein. In some of these embodiments, the indehiscence-associated mutation is selected from one that results in: a) a tryptophan at a position corresponding to 29 of SEQ ID NO: 21; b) a serine at a position corresponding to 15 of SEQ ID NO: 21; and c) an aspartic acid at a position corresponding to 12 of SEQ ID NO: 21. In particular embodiments, genetically-modifying the Vanilla sp. genome comprises introducing each of the indehiscence-associated mutations of a), b), and c).

In other specific embodiments of the method, genetically-modifying the Vanilla sp. genome comprises introducing at least one indehiscence-associated mutation into the dehiscent gene encoding a Sh1 protein. In some of these embodiments, the indehiscence-associated mutation results in a threonine at a position corresponding to 113 of SEQ ID NO: 23.

In some embodiments, the Vanilla sp. plant is Vanilla planifolia, Vanilla pompona, or Vanilla odorata.

In still another aspect, the invention provides a genetically-modified Vanilla sp. plant cell having at least one indehiscence-associated mutation in at least one dehiscent gene or reduced expression of at least one dehiscent gene compared to a non-genetically-modified Vanilla sp. plant cell, wherein the genome of the genetically-modified Vanilla sp. plant cell comprises the at least one indehiscence-associated mutation in the at least one dehiscent gene or at least one genetic modification that reduces the expression of the at least one dehiscent gene compared to a non-genetically-modified Vanilla sp. plant cell, wherein the dehiscent gene encodes a Shatterproof, Indehiscent, Replumless, Adpg1, or Sh1 protein.

In particular embodiments, at least one copy of at least one dehiscent gene is disrupted or knocked out. In some of these embodiments, all copies of at least one dehiscent gene is disrupted or knocked out.

In other embodiments, the genetically-modified Vanilla sp. plant cell comprises at least one mutation of at least one dehiscent gene such that the activity of the encoded protein is reduced. In some of these embodiments, the at least one mutation comprises at least one missense mutation. In other embodiments, the at least one mutation comprises at least one nonsense mutation such that at least one dehiscent gene encodes a truncated protein.

In specific embodiments of the composition, the genetically-modified Vanilla sp. plant cell comprises at least one indehiscence-associated mutation in the dehiscent gene encoding a Shatterproof protein. In some of these embodiments, the indehiscence-associated mutation is selected from one that results in: a) a 149 at a position corresponding to 149 of SEQ ID NO: 15; and b) a tyrosine at a position corresponding to 165 of SEQ ID NO: 15. In particular embodiments, the genetically-modified Vanilla sp. plant cell comprises each of the indehiscence-associated mutations of a) and b).

In other specific embodiments of the composition, the genetically-modified Vanilla sp. plant cell comprises at least one indehiscence-associated mutation in the dehiscent gene encoding an Indehiscent protein. In some of these embodiments, the indehiscence-associated mutation is selected from one that results in: a) a serine inserted in between positions corresponding to 45 and 46 of SEQ ID NO: 17; and b) a proline at a position corresponding to 35 of SEQ ID NO: 17. In particular embodiments, the genetically-modified Vanilla sp. plant cell comprises each of the indehiscence-associated mutations of a), b), and c).

In still other specific embodiments of the composition, the genetically-modified Vanilla sp. plant cell comprises at least one indehiscence-associated mutation in the dehiscent gene encoding a Replumless protein. In some of these embodiments, the indehiscence-associated mutation results in a glycine at a position corresponding to 10 of SEQ ID NO: 19

In yet other specific embodiments of the composition, the genetically-modified Vanilla sp. plant cell comprises at least one indehiscence-associated mutation in the dehiscent gene encoding an Adpg1 protein. In some of these embodiments, the indehiscence-associated mutation is selected from one that results in: a) a tryptophan at a position corresponding to 29 of SEQ ID NO: 21; b) a serine at a position corresponding to 15 of SEQ ID NO: 21; and c) an aspartic acid at a position corresponding to 12 of SEQ ID NO: 21. In particular embodiments, the genetically-modified Vanilla sp. plant cell comprises each of the indehiscence-associated mutations of a), b), and c).

In other specific embodiments of the composition, the genetically-modified Vanilla sp. plant cell comprises at least one indehiscence-associated mutation in the dehiscent gene encoding a Sh1 protein. In some of these embodiments, the indehiscence-associated mutation results in a threonine at a position corresponding to 113 of SEQ ID NO: 23.

In some embodiments, the Vanilla sp. plant cell is Vanilla planifolia, Vanilla pompona, or Vanilla odorata. A Vanilla sp. plant or plant part comprising the genetically-modified Vanilla sp. plant cell is also provided. In some embodiments, the Vanilla sp. plant part comprises a bean. In some embodiments of the composition, the Vanilla sp. plant or plant part has reduced dehiscence compared to a Vanilla sp. plant or plant part not comprising the genetically-modified Vanilla sp. plant cell. An extract from the Vanilla sp. plant or plant part comprising the genetically-modified Vanilla sp. plant cell is also provided.

In another aspect, the invention provides a method for introducing at least one mexicana-associated mutation into at least one MADS-box gene or reducing the expression of at least one MADS-box gene in a Vanilla sp. plant cell by genetically-modifying the genome of the Vanilla sp. plant cell to introduce the at least one mexicana-associated mutation into the at least one MADS-box gene or to reduce the expression of the at least one MADS-box gene to generate a genetically-modified Vanilla sp. plant cell.

In particular embodiments of the method, the MADS-box gene encodes a MADS-box protein having the sequence of any one of SEQ ID NOs: 26, 28, 30, 32, and 34. In certain embodiments, at least one copy of at least one MADS-box gene is disrupted or knocked out. In some of these embodiments, all copies of at least one MADS-box gene is disrupted or knocked out.

In certain embodiments, genetically-modifying the Vanilla sp. genome comprises mutating at least one MADS-box gene such that the activity of the encoded protein is reduced. In some of these embodiments, mutating the gene comprises introducing at least one missense mutation. In other embodiments, mutating the gene comprises introducing at least one nonsense mutation such that the MADS-box gene encodes a truncated protein.

In some embodiments of the method, the Vanilla sp. plant cell is Vanilla planifolia, Vanilla x tahitensis, or Vanilla pompona. In certain embodiments, the Vanilla sp. plant cell is within a seed or a seed capsule. In particular embodiments, a Vanilla sp. plant comprising the genetically-modified Vanilla sp. plant cell has a flower comprising a rostellum of reduced size compared to a Vanilla sp. plant not comprising the genetically-modified Vanilla sp. plant cell. In some of these embodiments, the Vanilla sp. plant lacks a rostellum. In certain embodiments, the Vanilla sp. plant is capable of self-pollination.

In another aspect, the invention provides a method for producing a Vanilla sp. plant having at least one mexicana-associated mutation in at least one MADS-box gene or reduced expression of at least one MADS-box gene by: a) genetically-modifying the genome of a Vanilla sp. plant cell or plant part to introduce the at least one mexicana-associated mutation into the at least one MADS-box gene or to reduce the expression of the at least one MADS-box gene to generate a genetically-modified Vanilla sp. plant cell or plant part; and b) growing a plant from the genetically-modified Vanilla sp. plant cell or plant part, wherein the Vanilla sp. plant has the at least one mexicana-associated mutation in the at least one MADS-box gene or reduced expression of the at least one MADS-box gene compared to a control plant.

In some embodiments of the method, a flower of the Vanilla sp. plant has a rostellum is of reduced size compared to a control plant. In other embodiments, the Vanilla sp. plant lacks a rostellum. In certain embodiments, the Vanilla sp. plant is capable of self-pollination.

In particular embodiments of the method, the MADS-box gene encodes a MADS-box protein having the sequence of any one of SEQ ID NOs: 26, 28, 30, 32, and 34. In certain embodiments, at least one copy of at least one MADS-box gene is disrupted or knocked out. In some of these embodiments, all copies of at least one MADS-box gene is disrupted or knocked out.

In certain embodiments, genetically-modifying the Vanilla sp. genome comprises mutating at least one MADS-box gene such that the activity of the encoded protein is reduced. In some of these embodiments, mutating the gene comprises introducing at least one missense mutation. In other embodiments, mutating the gene comprises introducing at least one nonsense mutation such that the MADS-box gene encodes a truncated protein.

In some embodiments of the method, the Vanilla sp. is Vanilla planifolia, Vanilla x tahitensis, or Vanilla pompona.

In still another aspect, the invention provides a genetically-modified Vanilla sp. plant cell having at least one mexicana-associated mutation in at least one MADS-box gene or reduced expression of at least one MADS-box gene compared to a non-genetically-modified Vanilla sp. plant cell, wherein the genome of the genetically-modified plant cell comprises at least one mexicana-associated mutation in at least one gene encoding a MADS-box protein or at least one genetic modification that reduces the expression of at least one gene encoding a MADS-box protein compared to a non-genetically-modified Vanilla sp. plant cell.

In particular embodiments of the composition, the MADS-box gene encodes a MADS-box protein having the sequence of any one of SEQ ID NOs: 26, 28, 30, 32, and 34.

In certain embodiments, at least one copy of at least one MADS-box gene is disrupted or knocked out. In some of these embodiments, all copies of at least one MADS-box gene is disrupted or knocked out.

In certain embodiments, the genetically-modified Vanilla sp. plant cell comprises at least one mexicana-associated mutation that reduces the activity of the encoded protein. In some of these embodiments, the at least one mexicana-associated mutation comprises at least one missense mutation. In other embodiments, the at least one mexicana-associated mutation comprises a nonsense mutation that results in the MADS-box gene encoding a truncated protein.

In some embodiments of the composition, the genetically-modified Vanilla sp. plant cell is a genetically-modified Vanilla planifolia, Vanilla x tahitensis, or Vanilla pompona.

Also provided is a Vanilla sp. plant or plant part comprising the genetically-modified Vanilla sp. plant cell. In some embodiments of the composition, a flower of the Vanilla sp. plant comprising the genetically-modified Vanilla sp. plant cell has a rostellum of reduced size compared to a control plant. In other embodiments, the Vanilla sp. plant lacks a rostellum. In certain embodiments, the Vanilla sp. plant is capable of self-pollination.

The provided Vanilla sp. plant part can comprise a bean or a seed. An extract from the Vanilla sp. plant or plant part is also provided.

In another aspect, the invention provides a method for producing a Vanilla sp. plant cell comprising at least one heterologous sequence encoding a fungal resistance protein or at least one pompona-associated mutation within at least one endogenous inactive fungal resistance gene by introducing into a Vanilla sp. plant cell the at least one heterologous sequence encoding said fungal resistance protein or genetically-modifying the genome of a Vanilla sp. plant cell to introduce the at least one pompona-associated mutation within the at least one endogenous inactive fungal resistance gene such that the introduction of said at least one pompona-associated mutation in said endogenous inactive fungal resistance gene generates an active fungal resistance gene that encodes a fungal resistance protein.

In certain embodiments of the method wherein the Vanilla sp. plant cell comprises at least one heterologous sequence encoding a fungal resistance protein, the fungal resistance protein has an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 36, 38, and 40. In some of these embodiments, the inactive fungal resistance protein is mutated to comprise at least one of the amino acid residues selected from the group consisting of: a) any one of a glycine, glutamic acid, histidine, glutamic acid, threonine, serine, lysine, histidine, leucine, isoleucine, glycine, arginine, leucine, aspartic acid, aspartic acid, glycine, asparagine, methionine, methionine, aspartic acid, glutamine, aspartic acid, asparagine, alanine, and glycine at positions corresponding to 28, 82, 91, 113, 131, 132, 147, 193, 199, 207, 227, 246, 271, 318, 324, 333, 336, 367, 379, 380, 408, 433, 443, 460, and 462, respectively of SEQ ID NO: 36; b) any one of a glutamic acid, aspartic acid, glycine, methionine, methionine, threonine, isoleucine, lysine, arginine, lysine, asparagine, phenylalanine, lysine, proline, phenylalanine, lysine, and alanine at positions corresponding to 29, 107, 216, 229, 362, 404, 547, 574, 610, 638, 695, 706, 773, 840, 860, 870, and 889, respectively of SEQ ID NO: 38; and c) any one of an alanine, glycine, isoleucine, glutamic acid, alanine, serine, tyrosine, methionine, lysine, glutamine, lysine, and serine at positions corresponding to 91, 124, 227, 333, 381, 537, 555, 703, 716, 754, 758, and 768, respectively of SEQ ID NO: 40.

In some embodiments of the method, the Vanilla sp. plant cell has increased resistance to a fungus compared to a control plant cell. In particular embodiments, the Vanilla sp. plant cell is a Vanilla planifolia or Vanilla x tahitensis plant cell.

In particular embodiments of the method, the fungus is a Fusarium sp. In some of these embodiments, the Fusarium sp. is F. oxysporum f. sp. vanilla.

In another aspect, the invention provides a method for producing a Vanilla sp. plant having at least one heterologous sequence encoding a fungal resistance protein or at least one pompona-associated mutation within at least one endogenous inactive fungal resistance gene by introducing into a Vanilla sp. plant cell or plant part the at least one heterologous sequence encoding the fungal resistance protein or genetically-modifying the genome of a Vanilla sp. plant cell or plant part to introduce the at least one pompona-associated mutation within the at least one endogenous inactive fungal resistance gene such that the introduction of said at least one pompona-associated mutation in said endogenous inactive fungal resistance gene generates an active fungal resistance gene that encodes a fungal resistance protein, and growing a Vanilla sp. plant from the Vanilla sp. plant cell or plant part, wherein the Vanilla sp. plant has the at least one heterologous sequence encoding the fungal resistance protein or the at least one pompona-associated mutation within the at least one endogenous fungal resistance gene.

In certain embodiments of the method wherein the Vanilla sp. plant cell, plant part and plant comprise at least one heterologous sequence encoding a fungal resistance protein, the fungal resistance protein has an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 36, 38, and 40. In some of these embodiments, the inactive fungal resistance protein is mutated to comprise at least one of the amino acid residues selected from the group consisting of: a) any one of a glycine, glutamic acid, histidine, glutamic acid, threonine, serine, lysine, histidine, leucine, isoleucine, glycine, arginine, leucine, aspartic acid, aspartic acid, glycine, asparagine, methionine, methionine, aspartic acid, glutamine, aspartic acid, asparagine, alanine, and glycine at positions corresponding to 28, 82, 91, 113, 131, 132, 147, 193, 199, 207, 227, 246, 271, 318, 324, 333, 336, 367, 379, 380, 408, 433, 443, 460, and 462, respectively of SEQ ID NO: 36; b) any one of a glutamic acid, aspartic acid, glycine, methionine, methionine, threonine, isoleucine, lysine, arginine, lysine, asparagine, phenylalanine, lysine, proline, phenylalanine, lysine, and alanine at positions corresponding to 29, 107, 216, 229, 362, 404, 547, 574, 610, 638, 695, 706, 773, 840, 860, 870, and 889, respectively of SEQ ID NO: 38; and c) any one of an alanine, glycine, isoleucine, glutamic acid, alanine, serine, tyrosine, methionine, lysine, glutamine, lysine, and serine at positions corresponding to 91, 124, 227, 333, 381, 537, 555, 703, 716, 754, 758, and 768, respectively of SEQ ID NO: 40.

In some embodiments of the method, the Vanilla sp. plant has increased resistance to a fungus compared to a control plant. In particular embodiments, the plant is a Vanilla planifolia or Vanilla x tahitensis plant.

In particular embodiments of the method, the fungus is a Fusarium sp. In some of these embodiments, the Fusarium sp. is F. oxysporum f. sp. vanilla.

In still another aspect, the invention provides a Vanilla sp. plant cell comprising a heterologous sequence encoding a fungal resistance gene or the genome of the Vanilla sp. plant cell is genetically-modified to comprise a pompona-associated mutation within at least one endogenous fungal resistance gene.

In certain embodiments of the composition wherein the Vanilla sp. plant cell comprises at least one heterologous sequence encoding a fungal resistance protein, the fungal resistance protein has an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 36, 38, and 40. In some of these embodiments, the inactive fungal resistance protein is mutated to comprise at least one of the amino acid residues selected from the group consisting of: a) any one of a glycine, glutamic acid, histidine, glutamic acid, threonine, serine, lysine, histidine, leucine, isoleucine, glycine, arginine, leucine, aspartic acid, aspartic acid, glycine, asparagine, methionine, methionine, aspartic acid, glutamine, aspartic acid, asparagine, alanine, and glycine at positions corresponding to 28, 82, 91, 113, 131, 132, 147, 193, 199, 207, 227, 246, 271, 318, 324, 333, 336, 367, 379, 380, 408, 433, 443, 460, and 462, respectively of SEQ ID NO: 36; b) any one of a glutamic acid, aspartic acid, glycine, methionine, methionine, threonine, isoleucine, lysine, arginine, lysine, asparagine, phenylalanine, lysine, proline, phenylalanine, lysine, and alanine at positions corresponding to 29, 107, 216, 229, 362, 404, 547, 574, 610, 638, 695, 706, 773, 840, 860, 870, and 889, respectively of SEQ ID NO: 38; and c) any one of an alanine, glycine, isoleucine, glutamic acid, alanine, serine, tyrosine, methionine, lysine, glutamine, lysine, and serine at positions corresponding to 91, 124, 227, 333, 381, 537, 555, 703, 716, 754, 758, and 768, respectively of SEQ ID NO: 40. In some embodiments of the composition, the Vanilla sp. plant cell has increased resistance to a fungus compared to a control plant cell. In particular embodiments, the Vanilla sp. plant cell is a Vanilla planifolia or Vanilla x tahitensis plant cell.

In particular embodiments of the composition, the fungus is a Fusarium sp. In some of these embodiments, the Fusarium sp. is F. oxysporum f. sp. vanilla.

A Vanilla sp. plant or plant part comprising the Vanilla sp. plant cell, wherein the Vanilla sp. plant or plant part has increased resistance to a fungus, is provided. An extract from the Vanilla sp. plant or plant part is also provided. A bean or seed of the Vanilla sp. plant comprising the genetic modification or heterologous sequence is further provided.

In yet another aspect, the invention provides a method of creating a population of Vanilla sp. plants having at least two copies of a gene encoding a phenylalanine ammonia lyase (PAL) polypeptide in its genome, wherein the method comprises: a) obtaining at least one DNA sample from at least one plant within a first population of Vanilla sp. plants; b) detecting the presence of at least two copies of a gene encoding PAL within the DNA sample; c) selecting one or more Vanilla sp. plants from the first population of Vanilla sp. plants based on the presence of the two copies of the gene encoding PAL in the DNA sample from the Vanilla sp. plant; and d) crossing the selected Vanilla sp. plant with itself or another, different Vanilla sp. plant to produce a population of offspring wherein the offspring population comprises the at least two copies of the gene encoding PAL.

In some embodiments of the method, the offspring population exhibits increased levels of PAL polypeptide compared to a Vanilla sp. plant that has less than two copies of the gene encoding PAL. In particular embodiments, a plant or plant part of the population of Vanilla sp. plants comprises increased levels of vanillin or one or more precursors thereof compared to a control plant. In some of these embodiments, the precursor of vanillin comprises cinnamic acid.

In still another aspect, the invention provides a method of creating a population of Vanilla sp. plants having at least two copies of a gene encoding a cysteine protease-like protein (CPLP), wherein the method comprises: a) detecting the presence of at least two copies of a gene encoding CPLP within a DNA sample from at least one plant within a first population of Vanilla sp. plants; b) selecting one or more Vanilla sp. plants from the first population of Vanilla sp. plants based on the presence of the two copies of the gene encoding CPLP in the DNA sample from the Vanilla sp. plant; and c) crossing the selected Vanilla sp. plant with itself or another, different Vanilla sp. plant to produce a population of offspring wherein the offspring population comprises the at least two copies of the gene encoding CPLP.

In some embodiments of the method, the offspring population exhibits increased levels of CPLP compared to a Vanilla sp. plant that has less than two copies of the gene encoding CPLP. In particular embodiments, a plant or plant part of the population of Vanilla sp. plants comprises increased levels of vanillin or one or more precursors thereof compared to a control plant. In some of these embodiments, the precursor of vanillin comprises 4-hydroxybenzaldehyde.

In another aspect, the invention provides a method of creating a population of Vanilla sp. plants having at least one indehiscence-associated mutation in at least one dehiscent gene, wherein the method comprises: a) detecting the presence of at least one indehiscence-associated mutation in at least one dehiscent gene within a DNA sample from at least one plant within a first population of Vanilla sp. plants, wherein the dehiscent gene encodes a Shatterproof, Indehiscent, Replumless, Adpg1, or Sh1 protein; b) selecting one or more Vanilla sp. plants from the first population of Vanilla sp. plants based on the presence of the at least one indehiscence-associated mutation in at least one dehiscent gene in the DNA sample from said Vanilla sp. plant; and c) crossing the selected Vanilla sp. plant with itself or another, different Vanilla sp. plant to produce a population of offspring wherein the offspring population comprises the at least one indehiscence-associated mutation in at least one dehiscent gene.

In some embodiments of the method, the offspring population exhibits reduced dehiscence compared to a Vanilla sp. plant that lacks at least one indehiscence-associated mutation in at least one dehiscent gene.

In yet another aspect, the invention provides a method of creating a population of Vanilla sp. plants having at least one mexicana-associated mutation in at least one MADS-box gene, wherein the method comprises: a) detecting the presence of at least one mexicana-associated mutation in at least one MADS-box gene within a DNA sample from at least one plant within a first population of Vanilla sp. plants; b) selecting one or more Vanilla sp. plants from the first population of Vanilla sp. plants based on the presence of the at least one mexicana-associated mutation in at least one MADS-box gene in the DNA sample from the Vanilla sp. plant; and c) crossing the selected Vanilla sp. plant with itself or another, different Vanilla sp. plant to produce a population of offspring wherein the offspring population comprises the at least one mexicana-associated mutation in at least one MADS-box gene.

In certain embodiments of the method, the offspring population has a rostellum of reduced size compared to a control Vanilla sp. plant or lacks a rostellum. In particular embodiments, the Vanilla sp. plant is capable of self-pollination.

In another aspect, the invention provides a method of creating a population of Vanilla sp. plants comprising a heterologous sequence encoding a fungal resistance gene or a pompona-associated mutation within at least one endogenous fungal resistance gene, wherein the method comprises: a) detecting the presence of a heterologous sequence encoding a fungal resistance protein or at least one pompona-associated mutation in at least one endogenous fungal resistance gene within a DNA sample from at least one plant within a first population of Vanilla sp. plants; b) selecting one or more Vanilla sp. plants from the first population of Vanilla sp. plants based on the presence of the heterologous sequence encoding a fungal resistance protein or the at least one pompona-associated mutation in at least one endogenous fungal resistance gene in the DNA sample from said Vanilla sp. plant; and c) crossing the selected Vanilla sp. plant with itself or another, different Vanilla sp. plant to produce a population of offspring wherein the offspring population comprises the heterologous sequence encoding a fungal resistance protein or the at least one pompona-associated mutation in at least one endogenous fungal resistance gene.

In some embodiments of the method, the offspring population has increased resistance to a fungus compared to a control Vanilla sp. plant. In certain embodiments, the fungus is a Fusarium sp. In some of these embodiments, the Fusarium sp. is F. oxysporum f. sp. vanilla.

### BRIEF DESCRIPTION OF THE FIGURES

FIG. 1 provides images of the vanilla plant, flowers, and beans. FIG. 1A is an image of a V. planifolia plant showing vine and immature bean clusters on racemes. FIG. 1B is an image of a V. planifolia flower showing typical cream green color. FIG. 1C shows cured vanilla beans with typical brown color and bundled for export. Flowers of resequenced species are shown in FIG. 1D (V. x tahitensis), FIG. 1E (V. pompona), and FIG. 1F (V. mexicana). Flower anatomical differences between V. planifolia (FIG. 1G) and V. mexicana (FIG. 1H) are shown with pollinia (with pollen), rostellum (if present), and stigmata as indicated. The lower petal was partially removed from V. planifolia and entirely removed from V. mexicana to show the presence and absence of the rostellum, respectively.

FIG. 2 provides chromosome counts and flow cytometry of V. planifolia ‘Daphna,’ demonstrating this accession is similar to other V. planifolia accessions. FIG. 2A provides a representative light microscopy image showing 28 V. planifolia chromosomes from a meristematic tip preparation. FIG. 2B shows a representative flow cytometry result from V. planifolia ‘Daphna’ meristem prepared with wheat leaf tissue that resulted in an estimated 2C=4.30 and 4C=8.47 pg similar to other published estimates. FIG. 2C shows a representative flow cytometry result from V. planifolia ‘Daphna’ young leaf tissue showing endoreduplication (2C=4.19 pg, 2E=5.27 pg, 4E=7.40 pg) with wheat leaf tissue as a size standard.

FIG. 3 provides a whole genome alignment between the phased, chromosome-level assembly and the previously published draft assembly Vapla0.1.4. Contigs in the Vapla0.1.4 assembly are ordered based on alignments to the phased assembly in this study. The portion of the Vapla0.1.4 assembly that have no aligned counterparts are thus shown towards the top of the dot plot.

FIG. 4 shows the V. planifolia ‘Daphna’ genomics analyses. FIG. 4A provides a histogram of intron lengths from annotated genes. FIG. 4B shows a distribution of synonymous substitutions per synonymous site (Ks) between syntenic gene pairs for pairwise genome comparisons. Gene pairs were based on syntenic gene anchor pairs, either as inferred orthologs when comparing two different genomes, or as inferred paralogs within a given genome when comparing within the same genome. FIG. 4C shows the length distribution of the ONT raw reads used to construct the genome assembly. Per base quality average 8.9, min 1.0, max 28.0. Red line denotes the mean of the distribution. FIG. 4D shows V. planifolia ‘Daphna’ K-mer distributions for various k based on Illumina reads. FIG. 4E shows SNPeff output showing the location of identified DNA variants and predicted impacts. FIG. 4F provides a Hi-C contact heatmap illustrating the strong diagonalization of the signal within chromosomes. The genome was divided into 500 kb bins and the number of Hi-C links are counted between all pairwise bins. Darker color indicates a higher number of Hi-C links. The contact map was obtained after correction.

FIG. 5 is a schematic of a chromosome-level, fully phased genome that was assembled for V. planifolia ‘Daphna’. FIG. 5A shows a Circos diagram depicting relationships of V. planifolia ‘Daphna’ A (left) and B (right) haplotypes with homologous blocks across haplotypes being connected with lines. The outer track (A) represents chromosomes with units in megabases. Interior tracks include (B) gene density, (C) DNA transposon coverage, and (D) retrotransposons coverage (green: LINEs and red: LTRs). FIG. 5B provides the depth of short read alignments from resequenced V. planifolia, V. x tahitensis, and V. pompona accessions on ‘Daphna’ haplotypes A and B. Each dot in the depth plot shows the median read depth per 1 Mb tiling window across the genome. Both the horizontal line and the number at the top of each track indicate the median read depth for each chromosome.

FIG. 6 shows the transcript abundance for 11,208 gene pairs identified by OrthoFinder analysis with a 1:1 ratio between haplotypes A and B. Data represents log 2 (FPKM haplotype B/FPKM haplotype A) values. Numbers close to zero indicate similar transcript abundances for gene pairs. Six tissue specific RNA-seq datasets are shown and are from a previously published study using V. planifolia ‘Daphna’ (Rao et al. (2014) BMC Genomics 15, doi:10.1186/1471-2164-15-964).

FIG. 7 provides a phylogenetic tree of Vanilla and other selected taxa. The species tree is inferred by 26 single-copy orthogroups in the selected plant taxa. Scale bar on the internal nodes represent 95% Highest Posterior Density (HPD) confidence intervals. Circles on the branches represent inferred whole genome duplication events with the α^{O} event and T event in the Vanilla lineage further highlighted.

FIG. 8 shows the karyotype of the vanilla genome illustrating the pan-orchid ao genome duplication. Pairs of duplicated regions are highlighted with the same color to show their corresponding locations in the genome.

FIG. 9 depicts the V. planifolia ‘Daphna’ comparative genomics analyses. Exemplar local synteny patterns between sets of regions in Vanilla and Apostasia compared against the basal angiosperm genome Amborella. The quadruply conserved synteny patterns in both orchid genomes were consistent with the two whole genome duplication events, including the α^{O}-WGD event shared by all orchid species and τ-WGD event shared by most monocot taxa, illustrated as circles.

FIG. 10 shows a PCA plot from resequencing of accessions in this study and from previously reported GBS data (Hu et al. (2019) Sci. Rep. 9:3416). Groupings of vanilla species are shown by color and as labeled. The numbers in parentheses indicate the number of accessions found within the groupings.

FIG. 11 depicts the genetic distance tree of resequenced accessions from this study and other accessions as previously reported (Hu et al. (2019) Sci. Rep. 9:3416).

FIG. 12 depicts the proposed vanillin biosynthesis pathway and new insights from the ‘Daphna’ genome. FIG. 12A depicts the vanillin pathway as previously proposed (Yang et al. (2017) Phytochem. 139:33-46; Gallage et al. (2014) Nat. Commun. 5, doi:10.1038/ncomms5037; Dixon (2018) “Vanillin biosynthesis—still not as simple as it seems?” in Handbook of Vanilla Science and Technology, Wiley) and adapted from a previous publication (Yang et al. (2017). The potential roles of CPLP and VpVan are indicated in gray boxes. FIG. 12B shows alignments of ‘Daphna’ RNA-seq reads on three CPLP homologs with examples of detected variants. FIG. 12C depicts the transcript abundance of genes involved in the proposed vanillin pathway across different developmental stages and tissue types.

FIG. 13 provides an analysis of the proposed vanillin pathway. FIG. 13A depicts the identification of ferulic acid/vanilla pathway intermediates from resequenced accessions. Short read transcript support that aligned to >80% of coding sequence at depths of at least 3× are shown by green circles for each accessions (red circles otherwise). FIG. 13B provides an alignment of three CPLP genes from the ‘Daphna’ genome with the published CPLP (aka VpVan) sequence. The alignments include both haplotype B paralogs (Vpl_s027Bg25938 and Vpl_s027Bg25947) and the alternative allele on haplotype A (Vpl_s027Ag26221). The nucleotide genomic sequences with predicted CDS for each gene as supported by a previous RNA-seq study (Rao et al. (2014) BMC Genomics 15, doi:10.1186/1471-2164-15-964) are provided. Primers used in previous studies are shown in red (Yang et al. (2017) Phytochem. 139:33-46), blue (Gallage et al. (2014) Nat. Commun. 5, doi:10.1038/ncomms5037), and green (Fock-Bastide et al. (2014) Plant Physiol. Biochem. 74:304-314). FIG. 13C provides a predicted protein alignment of CPLP sequences including previously published antibody probe (blue) (Gallage et al. (2018) Plant and Cell Physiol. 59:304-318). Figures created with Geneious v11.1.

FIG. 14 depicts the phylogeny of dehiscence and shattering-related protein sequences. Shown are genes from the ‘Daphna’ genome indicated by the Vpl designation. Also shown are selected dehiscence and shattering-related genes from A. thaliana, soy (G. max), and rice (O. sativa).

FIG. 15 shows the MADS-box genes in V. planifolia ‘Daphna’. A phylogenetic tree of MADS-box genes including Type 1 (MADS) and Type II (MADS, Intervening, Keratin-like, and C-terminal domains) MADS-box are shown.

FIG. 16 provides a sequence alignment between the PAL protein amino acid sequence (set forth as SEQ ID NO: 1) encoded by the functional Vpl_s453Bg28354 allele and the amino acid sequences encoded by the three non-functional alleles Vpl03Ag07441.1 (SEQ ID NO: 3), Vpl03Ag07445.1 (SEQ ID NO: 5), and Vpl_03Bg07223 (SEQ ID NO: 7).

FIG. 17 provides non-limiting examples of methods for increasing the expression of the Vpl_s453Bg28354 allele in a Vanilla sp. plant. FIG. 17A depicts a binary expression construct for Agrobacterium-mediated transformation of a Vanilla sp. comprising the neomycin phosphotransferase II (NPTII) gene driven by the 35S promoter and the Vpl_s453Bg28354 allele operably linked to the Cestrum yellow leaf curling virus (CmYLCV) promoter. FIG. 17B illustrates a similar binary expression construct wherein the Vpl_s453Bg28354 allele is operably linked to a pod, mesocarp, placenta, or seed-specific promoter. FIG. 17C shows a construct for gene editing via Agrobacterium-mediated transformation with a MAD7 nuclease that targets Vpl_s453Bg28354 allele promoter. The construct comprises the NPTII gene driven by the 35S promoter and the MAD7 nuclease expression driven by the CmYLCV promoter, as well as a cassette expressing a crRNA specific for targeting MAD7 to a region of the Vpl_s453Bg28354 allele promoter, the expression of which is regulated by the rice Pol III promoter, OsU6b. FIG. 17D illustrates a similar construct wherein the MAD7 is substituted for an engineered homing endonuclease (HEn nuclease) that targets the Vpl_s453Bg28354 allele promoter, and no crRNA expression cassette is needed. FIG. 17E illustrates a gene editing approach used to exchange the endogenous Vpl_s453Bg28354 allele promoter with a modified version that has higher expression levels or another, higher-expressing promoter. mRNA for at least one MAD7 or a HEn targeting the 5′ and 3′ boundaries of the native promoter is introduced into a protoplast, along with a double-stranded donor DNA for the targeted exchange via homology-directed recombination. FIG. 17F provides constructs for substitution of the endogenous Vpl_s453Bg28354 allele using a geminiviral payload in a binary backbone. The binary payload comprises the NPTII selection marker, the expression of which is driven by the 35S promoter, one or more HEn nuclease(s) targeting the DNA flanking the native Vpl_s453Bg28354 allele promoter operably linked to the CmYLCV promoter, and a modified or new promoter flanked by homology arms and the long-inverted repeats of the Geminivirus. This construct is delivered via Agrobacterium in trans with an additional construct comprising the Geminiviral Rep/RepA protein.

FIG. 18 provides non-limiting examples of methods for increasing the expression of the Vpl_s027Bg25947 and Vpl_s027Bg25938 alleles in a Vanilla sp. plant. FIG. 18A depicts a binary expression construct for Agrobacterium-mediated transformation of a Vanilla sp. comprising the neomycin phosphotransferase II (NPTII) gene driven by the 35S promoter and the Vpl_s027Bg25947 and/or Vpl_s027Bg25938 allele operably linked to the Cestrum yellow leaf curling virus (CmYLCV) promoter. FIG. 18B illustrates a similar binary expression construct wherein the Vpl_s027Bg25947 and/or Vpl_s027Bg25938 allele is operably linked to a pod, mesocarp, placenta, or seed-specific promoter. FIG. 18C shows a construct for gene editing via Agrobacterium-mediated transformation with a MAD7 nuclease that targets Vpl_s027Bg25947 and/or Vpl_s027Bg25938 allele promoter. The construct comprises the NPTII gene driven by the 35S promoter and the MAD7 nuclease expression driven by the CmYLCV promoter, as well as a cassette expressing a crRNA specific for targeting MAD7 to a region of the Vpl_s027Bg25947 and/or Vpl_s027Bg25938 allele promoter, the expression of which is regulated by the rice Pol III promoter, OsU6b. FIG. 18D illustrates a similar construct wherein the MAD7 is substituted for an engineered homing endonuclease (HEn nuclease) that targets the Vpl_s027Bg25947 and/or Vpl_s027Bg25938 allele promoter, and no crRNA expression cassette is needed. FIG. 18E illustrates a gene editing approach used to exchange the endogenous Vpl_s027Bg25947 and/or Vpl_s027Bg25938 allele promoter with a modified version that has higher expression levels or another, higher-expressing promoter. mRNA for at least one MAD7 or a HEn targeting the 5′ and 3′ boundaries of the native promoter is introduced into a protoplast, along with a double-stranded donor DNA for the targeted exchange via homology-directed recombination. FIG. 18F provides constructs for substitution of the endogenous Vpl_s027Bg25947 and/or Vpl_s027Bg25938 allele using a geminiviral payload in a binary backbone. The binary payload comprises the NPTII selection marker, the expression of which is driven by the 35S promoter, one or more HEn nuclease(s) targeting the DNA flanking the native Vpl_s027Bg25947 and/or Vpl_s027Bg25938 allele promoter operably linked to the CmYLCV promoter, and a modified or new promoter flanked by homology arms and the long-inverted repeats of the Geminivirus. This construct is delivered via Agrobacterium in trans with an additional construct comprising the Geminiviral Rep/RepA protein.

FIG. 19 illustrates methods for reducing or knocking out the expression of dehiscent genes (Vpl06Ag12707.1, Vpl09Ag19274.1, Vp101Ag01494.1, Vpl06Ag13482.1, and/or Vpl07Ag14471.1) or MADS-box genes (Vpl04Ag09199.1, Vpl06Ag12707.1, Vpl06Ag12680.1, Vp110Ag20060.1, Vp101Ag00567.1) using MAD7 or HEn nuclease(s) that result in a frame shift or nonsense mutation that disrupts normal gene function. Constructs comprising the NPTII selection marker gene regulated by the 35S promoter, the MAD7 nuclease sequence operably linked to the CmYLCV promoter, and a crRNA targeting the MAD7 to the dehiscent or MADS-box gene, the expression of which is controlled by the OsU6b promoter can be used. Alternatively, a binary payload construct comprising the 35S-NPTII expression cassette, and a HEn nuclease sequence targeting the dehiscent or MADS-box gene, regulated by the CmYLCV promoter is used. Protoplasts are transfected with mRNA coding for the MAD7 or HEn nuclease, followed by regeneration of plants from the protoplasts.

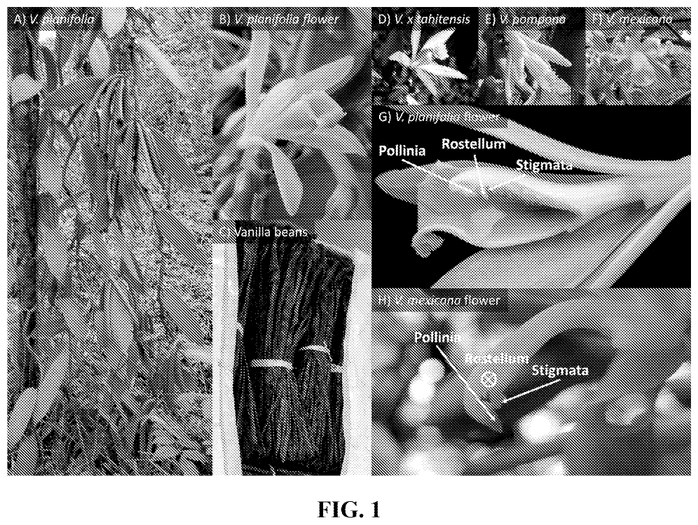

### BRIEF DESCRIPTION OF THE SEQUENCES

SEQ ID NO: 1 sets forth the amino acid sequence of the Vanilla planifolia ‘Daphna’ phenylalanine ammonia lyase enzyme encoded by the Vpl_s453Bg28354 allele.

SEQ ID NO: 2 sets forth the nucleic acid sequence of the Vpl_s453Bg28354 allele encoding the Vanilla planifolia ‘Daphna’ phenylalanine ammonia lyase enzyme.

SEQ ID NO: 3 sets forth the amino acid sequence encoded by the Vanilla planifolia ‘Daphna’ Vpl03Ag07441.1 allele.

SEQ ID NO: 4 sets forth the nucleic acid sequence of the Vanilla planifolia ‘Daphna’ Vpl03Ag07441.1 allele.

SEQ ID NO: 5 sets forth the amino acid sequence encoded by the Vanilla planifolia ‘Daphna’ Vpl03Ag07445.1 allele.

SEQ ID NO: 6 sets forth the nucleic acid sequence of the Vanilla planifolia ‘Daphna’ Vpl03Ag07445.1 allele.

SEQ ID NO: 7 sets forth the amino acid sequence encoded by the Vanilla planifolia ‘Daphna’ Vp103Bg07223.1 allele.

SEQ ID NO: 8 sets forth the nucleic acid sequence of the Vanilla planifolia ‘Daphna’ Vp103Bg07223.1 allele.

SEQ ID NO: 9 sets forth the amino acid sequence of the Vanilla planifolia ‘Daphna’ cysteine protease-like protein (CPLP) enzyme encoded by the Vpl_s027Bg25947 allele.

SEQ ID NO: 10 sets forth the nucleic acid sequence of the Vpl_s027Bg25947 allele encoding the Vanilla planifolia ‘Daphna’ CPLP enzyme.

SEQ ID NO: 11 sets forth the amino acid sequence of the Vanilla planifolia ‘Daphna’ CPLP enzyme encoded by the Vpl_s027Bg25938 allele.

SEQ ID NO: 12 sets forth the nucleic acid sequence of the Vpl_s027Bg25938 allele encoding the Vanilla planifolia ‘Daphna’ CPLP enzyme.

SEQ ID NO: 13 sets forth the amino acid sequence encoded by the Vanilla planifolia ‘Daphna’ Vpl_s027Ag26221.1 allele.

SEQ ID NO: 14 sets forth the nucleic acid sequence of the Vanilla planifolia ‘Daphna’ Vpl_s027Ag26221.1 allele.

SEQ ID NO: 15 sets forth the amino acid sequence of the Vanilla planifolia ‘Daphna’ Shatterproof protein encoded by the Vp106Ag12707.1 allele.

SEQ ID NO: 16 sets forth the nucleic acid sequence of the Vp106Ag12707.1 allele encoding the Vanilla planifolia ‘Daphna’ Shatterproof protein.

SEQ ID NO: 17 sets forth the amino acid sequence of the Vanilla planifolia ‘Daphna’ Indehiscent protein encoded by the Vp109Ag19274.1 allele.

SEQ ID NO: 18 sets forth the nucleic acid sequence of the Vp109Ag19274.1 allele encoding the Vanilla planifolia ‘Daphna’ Indehiscent protein.

SEQ ID NO: 19 sets forth the amino acid sequence of the Vanilla planifolia ‘Daphna’ Replumless protein encoded by the Vp101Ag01494.1 allele.

SEQ ID NO: 20 sets forth the nucleic acid sequence of the Vp101Ag01494.1 allele encoding the Vanilla planifolia ‘Daphna’ Replumless protein.

SEQ ID NO: 21 sets forth the amino acid sequence of the Vanilla planifolia ‘Daphna’ Arabidopsis dehiscence zone polygalacturonase1 (Adpg1) protein encoded by the Vpl06Ag13482.1 allele.

SEQ ID NO: 22 sets forth the nucleic acid sequence of the Vpl06Ag13482.1 allele encoding the Vanilla planifolia ‘Daphna’ Adpg1 protein.

SEQ ID NO: 23 sets forth the amino acid sequence of the Vanilla planifolia ‘Daphna’ Shattering1 (Sh1) protein encoded by the Vpl07Ag14471.1 allele.

SEQ ID NO: 24 sets forth the nucleic acid sequence of the Vpl07Ag14471.1 allele encoding the Vanilla planifolia ‘Daphna’ Sh1 protein.

SEQ ID NO: 25 sets forth the nucleic acid sequence of an example of a MADS-box domain from protein encoded by the Vanilla planifolia ‘Daphna’ Vpl04Ag08481.1 allele.

SEQ ID NO: 26 sets forth the amino acid sequence of the Vanilla planifolia ‘Daphna’ MADS-box protein encoded by the Vpl04Ag09199.1 allele.

SEQ ID NO: 27 sets forth the nucleic acid sequence of the Vpl04Ag09199.1 allele encoding a Vanilla planifolia ‘Daphna’ MADS-box protein.

SEQ ID NO: 28 sets forth the amino acid sequence of the Vanilla planifolia ‘Daphna’ MADS-box protein encoded by the Vpl06Ag12707.1 allele.

SEQ ID NO: 29 sets forth the nucleic acid sequence of the Vpl06Ag12707.1 allele encoding a Vanilla planifolia ‘Daphna’ MADS-box protein.

SEQ ID NO: 30 sets forth the amino acid sequence of the Vanilla planifolia ‘Daphna’ MADS-box protein encoded by the Vpl06Ag12680.1 allele.

SEQ ID NO: 31 sets forth the nucleic acid sequence of the Vpl06Ag12680.1 allele encoding a Vanilla planifolia ‘Daphna’ MADS-box protein.

SEQ ID NO: 32 sets forth the amino acid sequence of the Vanilla planifolia ‘Daphna’ MADS-box protein encoded by the Vpl10Ag20060.1 allele.

SEQ ID NO: 33 sets forth the nucleic acid sequence of the Vpl10Ag20060.1 allele encoding a Vanilla planifolia ‘Daphna’ MADS-box protein.

SEQ ID NO: 34 sets forth the amino acid sequence of the Vanilla planifolia ‘Daphna’ MADS-box protein encoded by the Vp101Ag00567.1 allele.

SEQ ID NO: 35 sets forth the nucleic acid sequence of the Vp101Ag00567.1 allele encoding a Vanilla planifolia ‘Daphna’ MADS-box protein.

SEQ ID NO: 36 sets forth the amino acid sequence of the Vanilla planifolia ‘Daphna’ inactive fungal resistance protein encoded by the Vpl02Ag05172.1 allele.

SEQ ID NO: 37 sets forth the nucleic acid sequence of the Vpl02Ag05172.1 allele encoding a Vanilla planifolia ‘Daphna’ inactive fungal resistance protein.

SEQ ID NO: 38 sets forth the amino acid sequence of the Vanilla planifolia ‘Daphna’ inactive fungal resistance protein encoded by the Vpl14Ag25867.1 allele.

SEQ ID NO: 39 sets forth the nucleic acid sequence of the Vpl14Ag25867.1 allele encoding a Vanilla planifolia ‘Daphna’ inactive fungal resistance protein.

SEQ ID NO: 40 sets forth the amino acid sequence of the Vanilla planifolia ‘Daphna’ inactive fungal resistance protein encoded by the Vpl_s056Ag26537.1 allele.

SEQ ID NO: 41 sets forth the nucleic acid sequence of the Vpl_s056Ag26537.1 allele encoding a Vanilla planifolia ‘Daphna’ inactive fungal resistance protein.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells. Further, the term “a gene” may include a plurality of genes, including a group of several genes.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “gene” refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants. The term “gene” can encompass both introns and exons.

As used herein, the term “allele” refers to one of two or more variant forms of a gene.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing two or more amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and can be provided in the form of an RNA sequence, a complementary polynucleotide sequence (cDNA), a genomic polynucleotide sequence and/or a composite polynucleotide sequences (e.g., a combination of the above).

As used herein, the term “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

As used herein, the term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “expression” or “expressing” refers to the transcription and/or translation of a particular nucleotide sequence driven by a promoter. As used herein, the terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild type plant. An exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “exon” in reference to a gene refers to the segment of the gene that codes for a protein or a segment thereof. In contrast, the term “intron” refers to the segment of a gene that does not code for proteins and interrupts the coding sequence.

As used herein, the term “endogenous” in reference to a gene or nucleotide sequence or protein is intended to mean a gene or nucleotide sequence or protein that is naturally comprised within or expressed by a cell. Endogenous genes can include genes that naturally occur in the cell of a plant, but that have been modified in the genome of the cell without insertion or replacement of a heterologous gene that is from another plant species or another location within the genome of the modified cell.

As used herein, the term “genomic locus” refers to a specific, fixed location on a chromosome where a particular gene or other DNA sequence is located.

As used herein, the term “diploid genome” refers to the genetic makeup of an organism comprising two complete sets of chromosomes, with one set inherited from each parent. As used herein, the term “polyploidy” or “polyploid genome” refers to the condition or a genome in which a normally diploid cell or organism acquires one or more additional sets of chromosomes and thus has three or more times the haploid chromosome number.

As used herein, the term “homozygous” refers to that which has two copies of the same allele for a particular gene located at similar positions (genomic loci) on paired chromosomes.

As used herein, the term “heterozygous” refers to that which has two different alleles for a particular gene located at similar positions (genomic loci) on paired chromosomes.

As used herein, with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol.266:131-141; Altschul et al. (1997), Nucleic Acids Res. 25:3389-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

According to some embodiments, the identity is a global identity, i.e., an identity over the entire amino acid or nucleic acid sequences of the invention and not over portions thereof.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

An expression cassette can permit transcription of a particular polynucleotide sequence in a host cell (e.g., a plant cell). An expression cassette may be part of a plasmid, viral genome, or nucleic acid fragment. Typically, an expression cassette includes a polynucleotide to be transcribed, operably linked to a promoter. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to a viral vector. Viral vectors can include, without limitation, retroviral vectors, lentiviral vectors, adenoviral vectors, and adeno-associated viral vectors (AAV).

As used herein, the term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

As used herein, the term “operably linked” is intended to mean a functional linkage between two or more elements. For example, an operable linkage between a nucleic acid sequence encoding a protein as disclosed herein and a regulatory sequence (e.g., a promoter) is a functional link that allows for expression of the nucleic acid sequence encoding the protein. Operably linked elements may be contiguous or non-contiguous. When used to refer to the joining of two protein coding regions, by operably linked is intended that the coding regions are in the same reading frame.

As used herein, the term “vanillin” refers to an organic compound (4-hydroxy-3-methoxybenzaldehyde) that is a key phenolic flavor compound of the extract of the vanilla bean.

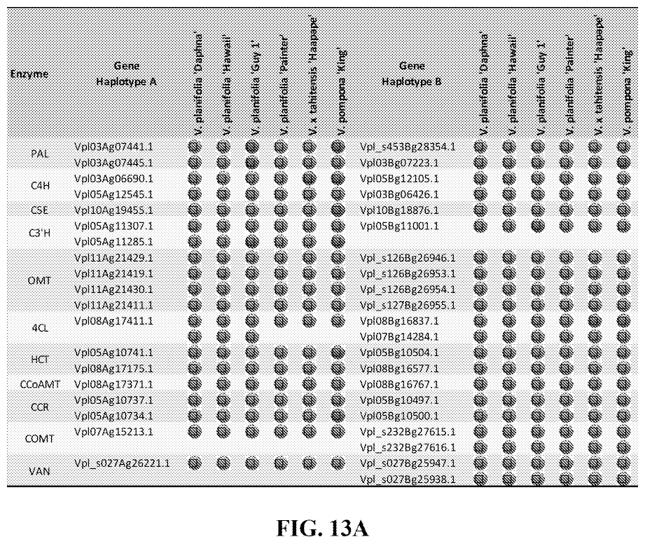

As used interchangeably herein, the term “vanillin synthesis pathway gene” or “vanillin pathway gene” refers to any gene that encodes an enzyme involved in the vanillin synthesis pathway (e.g., an enzyme catalyzing one or more reactions of the vanillin synthesis pathway, as proposed in FIG. 12A), or an active variant or homolog of that gene.

As used herein, the term “vanillin precursor” refers to any one of the organic compounds shown in FIG. 12A, as well as L-phenylalanine or other compounds determined to be upstream of vanillin in the biosynthetic pathway leading to vanillin, and including but not limited to trans-cinnamic acid, ferulic acid, 4-coumaric acid, and 4-hydroxybenzaldehyde.

As used herein, the term “phenylalanine ammonia lyase” or “PAL” refers to a polypeptide having the ability to catalyze the reaction of L-phenylalanine to trans-cinnamic acid and ammonia. PAL is a member of the ammonia lyase family, which cleaves carbon-nitrogen bonds and its enzymatic classification is EC 4.3.1.24. The cofactor 3,5-dihydro-5-methyldiene-4H-imidazol-4-one (MIO) is involved in the reaction catalyzed by PAL. The structure of various PAL enzymes has been determined (see, e.g., Calabrese et al. (2004) Biochemistry 43(36):11403-16; and Pilbak et al. (2006) The FEBS Journal 273(5):1004-19, each of which is incorporated by reference in its entirety). PAL is found in most plants, as well as some bacteria, yeast, and fungi. In some embodiments, the PAL enzyme utilized in the presently disclosed compositions and methods is that of Vanilla planifolia ‘Daphna’ set forth as SEQ ID NO: 1 or an active variant thereof (i.e., one that has PAL enzymatic activity). In some of these embodiments, the PAL gene utilized in the presently disclosed compositions and methods is that of V. planifolia ‘Daphna’ set forth as SEQ ID NO: 2 or an active variant thereof (i.e., one that encodes a polypeptide having PAL enzymatic activity). The term PAL gene also refers to naturally occurring DNA sequence variations of a PAL gene, such as a single nucleotide polymorphism (SNP). Exemplary SNPs may be found through the publicly accessible National Center for Biotechnology Information dbSNP Short Genetic Variations database.

As used herein, the term “cysteine protease-like protein” or “CPLP” or “vanillin synthase” or “VpVan” or “4HBS” refers to a polypeptide having the ability to catalyze the reaction of ferulic acid to vanillin, the reaction of 4-coumaric acid to 4-hydroxybenzaldehyde, or both. CPLP is a member of the aldehyde lyase family, which cleaves carbon-carbon bonds and its enzymatic classification is EC 4.1.2.41. The structure of various vanillin synthase enzymes has been determined (see, e.g., Bennet et al. (2008) Biochem J. 414(2):281-9; and Leonard et al. (2006) Acta Crystallogr D Biol Crystallogr. 62(Pt 12):1494-501, each of which is incorporated by reference in its entirety). In some embodiments, the CPLP enzyme utilized in the presently disclosed compositions and methods is that of Vanilla planifolia ‘Daphna’ set forth as SEQ ID NO: 9 or 11 or an active variant thereof (i.e., one that has at least one of the CPLP enzymatic activities described above). In some of these embodiments, the CPLP gene utilized in the presently disclosed compositions and methods is that of V. planifolia ‘Daphna’ set forth as SEQ ID NO: 10 or 12 or an active variant thereof (i.e., one that encodes a polypeptide having at least one of the CPLP enzymatic activities described above). The term CPLP gene also refers to naturally occurring DNA sequence variations of a CPLP gene, such as a single nucleotide polymorphism (SNP). Exemplary SNPs may be found through the publicly accessible National Center for Biotechnology Information dbSNP Short Genetic Variations database.

As used herein, the term “dehiscence” or “seed shattering” refers to the process by which fruit, seed pods or seed capsules are split open along a line of weakness (dehiscence zone). Seed pods or seed capsules that open in this manner are said to be dehiscent and those that do not open in this way are called indehiscent. Seed shattering occurs in many plant species including Arabidopsis, the Brassicaceae, tomato, soybean, many cereals and others and many of the pathways and proteins involved been described (see, e.g., Dong and Wang (2015) Front. Plant Sci., doi:10.3389/fpls.201500476; and Vittori et al. (2019) Genes 10, 68, doi:10.3390/genes10010068; each of which is herein incorporated by its entirety).

As used herein, the term “dehiscent gene” refers to a gene that is necessary for seed shattering and regulates the process of dehiscence.

As used herein, the term “Shatterproof protein” or “SHP” refers to a MADS-box transcription factor that controls the development of a dehiscence zone. The Arabidopsis Shatterproof-1 and -2 proteins were described by Liljegren et al. (2000) Nature 404:766-770, which is herein incorporated by reference in its entirety. SHP regulates the transcription of target genes Indehiscent and Alcatraz, which promote the correct differentiation of the lignification layer and the separation layer, respectively. According to the presently disclosed compositions and methods, the expression of a dehiscent gene, such as Shatterproof, is reduced or is genetically modified to introduce at least one indehiscence-associated mutation in order to reduce dehiscence in a plant such as a Vanilla sp. plant. In some embodiments, the gene encoding a Shatterproof protein utilized in the presently disclosed compositions and methods is that of Vanilla planifolia ‘Daphna’ that encodes the Shatterproof protein set forth as SEQ ID NO: 15. The term Shatterproof gene also refers to naturally occurring DNA sequence variations of a Shatterproof gene, such as a single nucleotide polymorphism (SNP). Exemplary SNPs may be found through the publicly accessible National Center for Biotechnology Information dbSNP Short Genetic Variations database.

As used herein, the term “Indehiscent protein” or “IND” refers to a basic helix-loop-helix (b-HLH) transcription factor that controls the development of a dehiscence zone. The Indehiscent protein is expressed in the dehiscence zone during late fruit development and its expression is regulated by Shatterproof protein(s). IND promotes the correct differentiation of the lignification layer. The Arabidopsis Indehiscent protein was described by Liljegren et al. (2004) Cell 116:843-853, which is herein incorporated by reference in its entirety. According to the presently disclosed compositions and methods, the expression of a dehiscent gene, such as Indehiscent, is reduced or is genetically modified to introduce at least one indehiscence-associated mutation in order to reduce dehiscence in a plant such as a Vanilla sp. plant. In some embodiments, the gene encoding an Indehiscent protein utilized in the presently disclosed compositions and methods is that of Vanilla planifolia ‘Daphna’ that encodes the Indehiscent protein set forth as SEQ ID NO: 17. The term Indehiscent gene also refers to naturally occurring DNA sequence variations of an Indehiscent gene, such as a single nucleotide polymorphism (SNP). Exemplary SNPs may be found through the publicly accessible National Center for Biotechnology Information dbSNP Short Genetic Variations database.

As used herein, the term “Replumless protein” or “RPL” refers to a homeodomain transcription factor that contributes to the specification of replum identity and restricts expression of SHP1/2 and IND to the dehiscence zone. The Replumless protein is expressed in the replum where it prevents ectopic lignification that is promoted by the valve margin genes SHP, and IND. The Arabidopsis Replumless protein was described by Roeder et al. (2003) Curr. Biol. 13:1630-1635, which is herein incorporated by reference in its entirety. According to the presently disclosed compositions and methods, the expression of a dehiscent gene, such as Replumless, is reduced or is genetically modified to introduce at least one indehiscence-associated mutation in order to reduce dehiscence in a plant such as a Vanilla sp. plant. In some embodiments, the gene encoding a Replumless protein utilized in the presently disclosed compositions and methods is that of Vanilla planifolia ‘Daphna’ that encodes the Replumless protein set forth as SEQ ID NO: 19. The term Replumless gene also refers to naturally occurring DNA sequence variations of a Replumless gene, such as a single nucleotide polymorphism (SNP). Exemplary SNPs may be found through the publicly accessible National Center for Biotechnology Information dbSNP Short Genetic Variations database.

As used herein, the term “Arabidopsis dehiscence zone polygalacturonase1” or “ADPG1” refers to plant specific endo-polygalacturonase that is expressed in the separation layer of flower organs and fruit dehiscence zones. Polygalacturonases are enzymatically classified as EC 3.2.1.15 and hydrolyze the alpha-1,4 glycosidic bonds between galacturonic acid residues. Polygalacturonan, whose major component is galacturonic acid, is a significant carbohydrate component of the pectin network that comprises plant cell walls. ADPG1 and the similar ADPG2 proteins are essential for enzymatic breakdown of pectin in the middle lamella, which promotes detachment of the valves from the replum in the separation layer prior to seed shattering. ADPGs are the final regulators of pod dehiscence in the separation layers. The ADPG1 and 2 proteins were described by Ogawa et al. (2009) Plant Cell 21:216-233, which is herein incorporated by reference in its entirety. According to the presently disclosed compositions and methods, the expression of a dehiscent gene, such as ADPG1, is reduced or is genetically modified to introduce at least one indehiscence-associated mutation in order to reduce dehiscence in a plant such as a Vanilla sp. plant. In some embodiments, the gene encoding an ADPG1 protein utilized in the presently disclosed compositions and methods is that of Vanilla planifolia ‘Daphna’ that encodes the ADPG1 protein set forth as SEQ ID NO: 21. The term ADPG1 gene also refers to naturally occurring DNA sequence variations of an ADPG1 gene, such as a single nucleotide polymorphism (SNP). Exemplary SNPs may be found through the publicly accessible National Center for Biotechnology Information dbSNP Short Genetic Variations database.

As used herein, the term “Shattering1” or “Sh1” refers to a transcription factor from the YABBY subfamily of zinc finger proteins. The Sorghum bicolor and Oryza sativa Sh1 proteins were described by Lin et al. (2012) Nat. Genet. 44:720-724, which is herein incorporated by reference in its entirety. According to the presently disclosed compositions and methods, the expression of a dehiscent gene, such as Sh1, is reduced or is genetically modified to introduce at least one indehiscence-associated mutation in order to reduce dehiscence in a plant such as a Vanilla sp. plant. In some embodiments, the gene encoding a Sh1 protein utilized in the presently disclosed compositions and methods is that of Vanilla planifolia ‘Daphna’ that encodes the Sh1 protein set forth as SEQ ID NO: 23. The term Sh1 gene also refers to naturally occurring DNA sequence variations of a Sh1 gene, such as a single nucleotide polymorphism (SNP). Exemplary SNPs may be found through the publicly accessible National Center for Biotechnology Information dbSNP Short Genetic Variations database.

As used herein, the term “MADS-box gene” refers to a gene encoding a protein comprising a DNA-binding MADS-box domain, which is named for the four initially identified members of this family: MCM1, AG, DEF, and SRF. The MADS-box domain, which can have a length of about 48 to about 60 amino acids, binds to DNA sequences of high similarity to the motif CC[A/T]₆GG called the CArG-box. A non-limiting example of a MADS-box domain is set forth as SEQ ID NO: 25. MADS-box genes include the floral homeotic MADS-box genes, such as AGAMOUS and DEFICIENS, that participate in the determination of floral organ identity according to the ABCDE model of flower development where regulators are responsible for initiating specific parts of the flower (sepals, petals, ovules, etc.) (see, e.g., Chen et al. (2012) Plant and Cell Physiol. 53:1053-1067, which is herein incorporated by reference in its entirety). The floral organ identity MADS-box genes have been divided into A, B, C, D, and E classes (Theissen (2001) Curr. Opin. Plant Biol. 4:75-85, which is herein incorporated by reference in its entirety). A- and E-class proteins are responsible for sepal development in the first floral whorl, the combination of A-, B-, and E-class proteins controls petal formation in the second whorl, the combination of B-, C-, and E-class proteins regulates stamen differentiation in the third whorl, the combination of C- and E-class proteins specifies carpel development in the fourth whorl, and the combination of D- and E-class proteins is required for ovule identity (Murai (2013) Plants 2:379-395, which is incorporated by reference in its entirety). AGAMOUS (C-class), SEEDSTICK (D-class), and other C- and D-class MADS-box genes have been associated with orchid flower development and specifically associated with rostellum tissues in two orchid species (see, e.g., Chen et al. (2012) and Skipper (2006) Gene 366:266-274, each of which is herein incorporated by reference in its entirety). In some embodiments, the MADS-box gene encodes a MADS-box protein having the sequence of any one of SEQ ID NOs: 26, 27, 28, 29, and 30 or an active variant thereof.

As used herein with respect to a parameter, the term “modulation” or “modulating” refers to a detectable positive or negative change in the parameter from a comparison control, e.g., an established normal or reference level of the parameter, or an established standard control. For example, as used herein, a method for modulating gene expression refers to a method disclosed herein that may elicit detectable positive or negative change in expression level of a gene (e.g., change in expression level of a gene in a cell, such as a plant cell) compared to a normal or reference expression level of the gene (e.g., expression level of the gene in the cell before the cell was subject to the said method, or a cell that has not been subject to the said method). Modulation of a parameter (e.g., modulation of gene expression) may refer to increase or decrease of the parameter (e.g., increase or decrease of gene expression). For example, a method for modulating gene expression may elicit detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) increase or positive change in expression level of a gene (e.g., expression level of a gene in a cell, such as a plant cell) compared to a normal or reference expression level of the gene (e.g., expression level of the gene in the cell before the cell was subject to the said method, or a cell that has not been subject to the said method). Alternatively, a method for modulating gene expression may elicit detectable (e.g., at least about 5%, 10%, 15%, 20%, 25%, 30%, 35%, 40%, 45%, 50%, 55%, 60%, 65%, 70%, 75%, 80%, 85%, 90%, 95%, 97%, 99%, or more) decrease, reduction or negative change in expression level of a gene (e.g., expression level of a gene in a cell, such as a plant cell) compared to a normal or reference expression level of the gene (e.g., expression level of the gene in the cell before the cell was subject to the said method, or a cell that has not been subject to the said method).

As used herein with respect to a parameter, the term “increased” or “increasing” or “increase” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) positive change in the parameter from a comparison control, e.g., an established normal or reference level of the parameter, or an established standard control. For example, increased production of vanillin or one or more precursors thereof in a plant may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) increase or positive change in production of vanillin or one or more precursors thereof in a plant compared to a control plant. Similarly, an increased level of vanillin or one or more precursors thereof in a plant part (e.g., bean) may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) increase or positive change in level of the vanillin or one or more precursors thereof in a part (e.g., bean) of a plant compared to a corresponding control plant part.

As used herein, the term “reduced expression” refers to any reduction in the expression of an endogenous gene when compared to a control cell. Such a reduction may be up to 5%, 10%, 20%, 30%, 40%, 50%, 60%, 70%, 80%, 90%, 95%, 96%, 97%, 98%, 99%, or up to 100% when compared to a control cell. Accordingly, the term “reduced” encompasses both a partial knockdown and a complete knockdown (i.e., knockout) of gene expression.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a donor template into a gene can result in no expression of an encoded protein, expression of a truncated protein, and/or expression of a protein that does not retain its wild-type function.

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, beans, leaves, flowers, branches, fruit, pulp, juice, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides or genomic modifications. Further provided is a processed plant product (e.g., extract) or byproduct produced from the plants or plant parts disclosed herein.

As used herein, the term “seed” refers to a flowering plant's unit of reproduction comprising a fertilized matured ovule that is capable of developing into another such plant.

As used herein, the term “seed capsule” or “pod” refers to a type of dry, rarely fleshy fruit that is comprised of two or more carpels. While the seed capsule can be dehiscent or indehiscent, most undomesticated plants having seed capsules are dehiscent.

As used herein, the term “bean” refers to both a seed capsule and its internal seeds. The bean can be split or intact. A non-limiting example is the beans of a Vanilla sp. plant. Unsplit, intact vanilla beans are desired, which are cured for commercial purposes, involving multiple steps of defined heat treatments that gradually reduce bean moisture content in order to develop the full vanilla aroma and stabilize the beans for transport. In some embodiments, the process of curing involves freezing of the beans prior to the heat/drying treatments and/or enzymatic treatments. Thus, a vanilla bean can be cured or uncured.

As used herein, the term “extract” refers to a composition comprising the active ingredient (e.g., aromatic ingredient) of a substance in concentrated form. In the non-limiting example of a vanilla extract, the extract comprises a more concentrated amount of at least the key aromatic component, vanillin, than within a bean. It should be noted that there are over one hundred additional aroma volatiles other than vanillin that are responsible for the vanilla flavor and often found in vanilla extract. The extract can be created using any method known in the art, but is most often created from intact vanilla beans that have been macerated and percolated in a solution of ethanol and water.

As used herein, the term “rostellum” refers to the flap-like organ that physically separates the male and female parts of an Orchidaceae flower. In some instances, the presence of a rostellum can prevent self-pollination.

As used herein, the term “self-pollination” refers to when the pollen from the anther is deposited on the stigma of the same flower, or another flower on the same plant.

As used herein, the term “fungal resistance” refers to the ability of a plant or plant part or plant cell to exhibit reduced symptoms associated with a fungal infection or a disease resulting therefrom compared to a control plant. Such symptoms include but are not limited to tissue necrosis, reduced biomass, or plant death.

In general terms, as used herein, the term “control plant” or “control plant part” or “control plant cell” refers to a plant or plant part or plant cell that has not been subject to the methods and compositions described herein. More specifically, as used herein, the term “a control plant” or “a control plant part” or “a control plant cell” refers to a plant, plant part, or plant cell that provides a reference point for measuring changes in a genotype or phenotype of a genetically-modified plant, plant part, or plant cell or a plant, plant part, or plant cell comprising a heterologous sequence. A control cell may comprise, for example: (a) a wild-type cell, i.e., of the same genotype as the starting material for the genetic alteration which resulted in the genetically-modified cell or prior to introduction of the heterologous sequence; (b) a cell of the same genotype as the genetically-modified cell or cell which has a heterologous sequence introduced, but which has been transformed with a null construct (i.e., with a construct which has no known effect on the trait of interest); or, (c) a cell genetically identical to the genetically-modified cell or cell comprising a heterologous sequence but which is not exposed to conditions or stimuli or further genetic modifications that would induce expression of altered genotype or phenotype.

As used herein, “genome editing” or “genome edits” or “modification” or “genetic modification” or “genetically-modifying” or “genetically-modified” or “engineered” or “engineering” or “genetic engineering” refers to any insertion, deletion, or substitution of an amino acid residue in the recombinant sequence relative to a reference sequence (e.g., a wild-type or a native sequence) or can refer to single strand cleavage, double strand cleavage or binding to a nucleic acid molecule in the genome of a cell or outside (e.g., plasmid) of the genome of a cell. “Modification” or “modifying” may indicate any detectable positive or negative effect on a process or on the function of a target, such as a promoter, a transcription factor gene, etc. Modification of a target (e.g., modification of a promoter) may refer to activation or inhibition of the target (e.g., activation or inhibition of the promoter). For example, modification of a promoter may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) activation or positive effect on the function of the promoter (e.g., function of the promoter in the genome of a cell, such as a plant cell) compared to a normal or reference level (e.g., function of the promoter in the genome of the cell before the cell was subject to the said method, or a cell that has not been subject to the said method). Alternatively, modification of a promoter may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 97%, 99%, or more) inhibition or negative effect on the function or activity of the promoter (e.g., function of the promoter in the genome of a cell, such as a plant cell) compared to a normal or reference level (e.g., function of the promoter in the genome of the cell before the cell was subject to the said method, or a cell that has not been subject to the said method).

Accordingly, “genetically-modified plant” or “genetically-modified plant part” or “genetically-modified cell” or “genetically-modified plant genome” refers to a plant or plant part or cell or genome that has been subject to one or more modifications described hereinabove.

As used herein, the term “mutation” refers to a change in a DNA sequence that may or may not translate to a change in an encoded amino acid sequence. The mutation may be a deletion, addition, or substitution.

As used herein, the term “missense mutation” refers to a mutation of a single base pair in a DNA sequence that translates into a substitution of a different amino acid in the encoded amino acid sequence. The encoded protein comprising the amino acid sequence with a missense mutation may or may not have the same activity as the protein without the missense mutation.

As used herein, the term “nonsense mutation” refers to a mutation of a single base pair in a DNA sequence that introduces a termination codon. In some such embodiments wherein a nonsense mutation is introduced into a protein-coding sequence, elongation of the protein is prematurely terminated due to the presence of the termination codon (as the result of the nonsense mutation), resulting in a truncated protein.

As used herein, the term “indehiscence-associated mutation” refers to a mutation introduced into a dehiscent gene of a dehiscent plant wherein the mutation that is introduced is derived from an indehiscent plant (e.g., V. x tahitensis) and represents a difference in sequence within the dehiscent gene between the dehiscent plant and the indehiscent plant.

In some embodiments, the introduction of one or more indehiscence-associated mutations into one or more dehiscent genes results in a reduction of the dehiscence rate of a plant (the percent of fruit or beans/pods of a plant that split) or renders the plant indehiscent. In certain embodiments, one or more indehiscence-associated mutations are introduced into one or more dehiscent genes selected from the group consisting of Shatterproof, Indehiscent, Replumless, Adpg1, and Sh1 genes.

As used herein, “mexicana-associated mutation” refers to a mutation that is derived from a Vanilla mexicana plant that represents a difference in sequence between the V. mexicana plant and the plant into which the mexicana-associated mutation is being introduced. V. mexicana plants lack a rostellum and in some embodiments, the mexicana-associated mutation is introduced into a plant that normally has a rostellum (e.g., V. planifolia, V. x tahitensis, or V. pompona) into a gene that regulates rostellum formation, including but not limited to, a MADS-box gene, a floral organ identity MADS-box gene, a C-class or D-class MADS box gene, an AGAMOUS gene, or a SEEDSTICK gene. In some of these embodiments, introduction of the mexicana-associated mutation(s) into a gene that regulates rostellum formation in a plant that normally has a rostellum leads to the formation of a rostellum that is reduced in size or absent altogether. In particular embodiments, the smaller rostellum or absence thereof as a result of the introduction of the mexicana-associated mutation(s) allows for self-pollination of a plant that otherwise lacks this ability.

As used herein, “pompona-associated mutation” refers to a mutation that is derived from a Vanilla pompona plant that represents a difference in sequence between the V. pompona plant and the plant into which the pompona-associated mutation is being introduced. V. pompona plants are naturally resistant to Fusarium sp. fungi such as F. oxysporum f. sp. vanilla. In some embodiments, the pompona-associated mutation is introduced into a plant that lacks resistance to fungi such as Fusarium sp. (e.g., F. oxysporum f. sp. vanilla), including but not limited to, V. planifolia, V. x tahitensis, or V. mexicana, into a fungal resistance gene. In some of these embodiments, introduction of the pompona-associated mutation(s) into a fungal resistance gene in a plant that normally lacks fungal resistance imparts fungal resistance to the plant.

As used herein, a “fungal resistance gene” refers to a gene that regulates or is present within a fungal resistance pathway and can contribute to fungal resistance in a plant. In some embodiments, the fungal resistance gene does not impart fungal resistance if particular mutations are present and is thus referred to herein as inactive. For example, an inactive fungal resistance gene within V. planifolia, V. x tahitensis, and V. mexicana does not naturally impart fungal resistance, but if mutation(s) are introduced from a homologous gene in a V. pompona plant, the fungal resistance gene can impart fungal resistance to the genetically-modified V. planifolia, V. x tahitensis, or V. mexicana plant.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the terms “nuclease” and “endonuclease” are used interchangeably to refer to naturally-occurring or engineered enzymes, which cleave a phosphodiester bond within a polynucleotide chain.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, EndI, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers.

In some embodiments, the compact TALEN comprises 16-22 TAL domain repeats. As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1 or Cas12a. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the term “megaTAL” refers to a single-chain endonuclease comprising a transcription activator-like effector (TALE) DNA binding domain with an engineered, sequence-specific homing endonuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, S1 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscouand Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S1 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease, or by a monomer of a TALEN or zinc finger nuclease.

As used herein, the terms “recognition sequence” or “recognition site” or “cleavage site” or “cleavage sequence” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI or Cas12a CRISPR nuclease is utilized, cleavage by the CRISPR complex comprising the same will result in 5′ overhangs and in certain embodiments, 5-nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the terms “target site” or “target sequence” refers to a region of the chromosomal DNA of a cell comprising a recognition sequence for a nuclease.

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other realvalues ≥0 and ≥2 if the variable is inherently continuous.

### 2.1 Principle of the Invention

A fully phased, chromosome-scale, reference genome for V. planifolia is presented herein that reveals haplotype-specific sequence and transcript abundance differences within the commercially-relevant vanillin pathway that impacts bean quality. Resequencing of related vanilla species identified genes that can impact productivity and post-harvest losses through pod dehiscence, flower anatomy, and disease resistance.

Identification of genes and allele-specific sequences that regulate various pathways and traits within Vanilla sp. plants allow for methods and compositions for improving various traits in plants, such as Vanilla sp. plants, by genetically-modifying the genome of plants or introducing heterologous sequences into the plants. Such traits that can be improved by the methods and compositions of the present invention include an increase in levels of vanillin or one or more precursors thereof, reducing dehiscence (i.e., seed shattering), reducing the size of a rostellum or eliminating its presence, and increasing fungal resistance. Also disclosed herein are genetically-modified plant cells, plant parts, or plants (such as a Vanilla sp. plant), extract from such plants, or plant parts (such as beans) from such plants, methods of producing such plants or progeny of such plants or a population of such plants or progeny thereof.

### 2.2 Plants, Plant Parts, and Plant Cells

In some embodiments described herein are methods for genetically-modifying or introducing heterologous sequences into vanilla plants (i.e., species of plants within the Vanilla genus), plant parts, or plant cells. Genetically-modified vanilla plants, vanilla plant parts, and vanilla plant cells or vanilla plants, vanilla plant parts, and vanilla plant cells comprising a heterologous sequence are also provided herein. Vanilla plants, also referred to as vanilla orchids are part of the Orchidaceae family of plants and are grown in tropical and subtropical regions. There are approximately 110 species within the Vanilla genus of flowering plants. Non-limited Vanilla species include V. albida, V. andamanica, V. aphylla, V. atropogon, V. bahiana, V. barbellata, V. chamissonis, V. claviculata, V. dilloniana, V. edwallii, V. humblotii, V. mexicana, V. moonii, V. odorata, V. phaeantha, V. pilifera, V. planifolia, V. poitaei, V. polylepis, V. pompona, V. raabii, V. roscheri, V. siamensis, V. somai, V. tahitensis (also referred to herein as V. x tahitensis), and V. walkeriae, and any variety of any of these species, such as V. pompona ‘Daphna’.

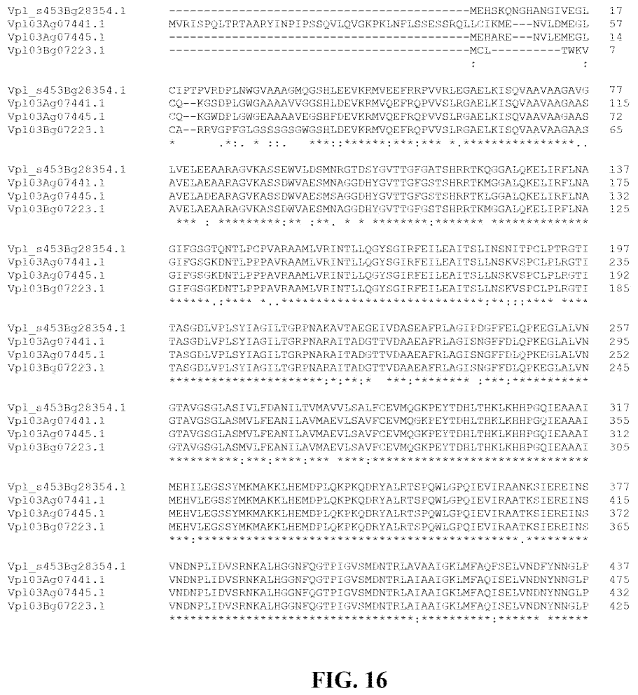

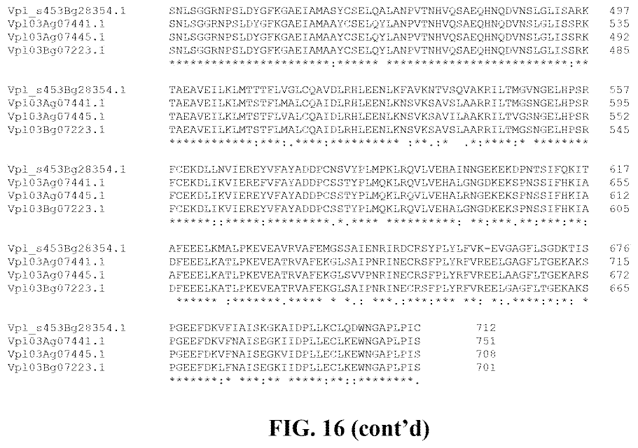

Described herein are genetically-modified plant cells having at least two copies of a gene encoding a phenylalanine lyase (PAL) polypeptide, wherein the genome comprises a genetic modification such that the genetically-modified plant cell comprises the at least two copies of the PAL gene. It was discovered herein that some genes within Vanilla sp. plants exhibit preferential allele expression. Thus, the two copies of the gene encoding a PAL polyeptide are alleles that actually express the PAL polypeptide. In some embodiments, the PAL polypeptide that is expressed from a specific PAL allele capable of expression has the amino acid sequence set forth as SEQ ID NO: 1 or an active variant thereof (i.e., one that has PAL enzymatic activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 1. In some of these embodiments, the active variant of SEQ ID NO: 1 retains one or more of the amino acid residues that are present in SEQ ID NO: 1, but not SEQ ID NO: 3, 5, and 7, as shown in the alignment provided in FIG. 16.

In certain embodiments, the coding region of the PAL allele capable of expression has the nucleotide sequence set forth as SEQ ID NO: 2 or an active variant thereof (i.e., one from which a polypeptide having PAL enzymatic activity is actually expressed) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 2.

In some of these embodiments, the two copies of the PAL gene can be within the endogenous genomic locus or within the genome, but outside of the naturally-occurring locus.

In certain embodiments, the genetically-modified plant cell, plant part, or plant comprises at least two copies of the PAL gene within a chromosome pair (one copy on each chromosome of the pair) of a diploid genome. The two copies of the PAL gene can be identical, making the genetically-modified plant cell, plant part, or plant homozygous for that particular PAL gene sequence. In contrast, the two copies of the PAL gene are not identical in sequence, making the genetically-modified plant cell, plant part, or plant heterozygous for that particular PAL gene sequence.

Alternatively, the genetically-modified plant cell, plant part, or plant comprises at least two copies of the PAL gene on a single chromosome.

The cell, plant part, or plant can be diploid or can exhibit polyploidy, which in some embodiments can be due to endoreduplication or partial endoreduplication. In the case of polyploidy, the genetically-modified plant cell, plant part, or plant can comprise two or more copies of a single chromosome or a part thereof and thus can comprise at least two copies of the PAL gene with one or more copies on a single chromosome, or one or more copies on two or more chromosomes.

In some of these embodiments, the genetically-modified plant cell, plant part, or plant has increased levels of PAL polypeptide and/or activity as compared to a non-genetically-modified plant cell, plant part, or plant. In some of these embodiments, the genetically-modified plant cell, plant part, or plant has at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more PAL polypeptide levels and/or activity compared to a non-genetically-modified plant cell, plant part, or plant.

In certain embodiments, the genetically-modified plant cell, plant part, or plant produces increased levels of vanillin or one or more vanillin precursor, such as cinnamic acid (e.g., trans-cinnamic acid), as compared to a non-genetically-modified plant cell, plant part, or plant. In some of these embodiments, the genetically-modified plant cell, plant part, or plant produces at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of vanillin or one or more vanillin precursor, such as cinnamic acid (e.g., trans-cinnamic acid) compared to a non-genetically-modified plant cell, plant part, or plant.

The genetically-modified plant cell can be within a plant part, such as a seed, seed capsule, or bean (comprising seeds and the seed capsule) or the genetically-modified plant part can be a genetically-modified seed, seed capsule, or bean. Genetically-modified seeds, seed capsules, or beans or an extract thereof can comprise increased levels of vanillin or one or more vanillin precursors, such as cinnamic acid (e.g., trans-cinnamic acid), as compared to non-genetically-modified seeds, seed capsules, or beans or an extract thereof. In some of these embodiments, the genetically-modified seeds, seed capsules, or beans or an extract thereof have at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of vanillin or one or more vanillin precursor, such as cinnamic acid (e.g., trans-cinnamic acid) compared to a non-genetically-modified seed, seed capsule, or bean or extract thereof. In some of these embodiments wherein the genetically-modified seed, seed capsule, or bean comprises increased levels of vanillin or one or more vanillin precursors, the seed, seed capsule, or bean is green, has been freshly picked or has been cured on or off the plant as described elsewhere herein. Thus, the uncured genetically-modified vanilla seed, seed capsule or bean or an extract thereof can comprise increased levels of vanillin or one or more vanillin precursors or the curing process might be necessary for the genetically-modified vanilla seed, seed capsule, or bean or extract thereof to exhibit increased levels of vanillin or one or more vanillin precursors compared to a similarly processed control, non-genetically-modified vanilla seed, seed capsule, or bean or extract thereof.

Also described herein are genetically-modified plant cells having at least two copies of a gene encoding a cysteine protease-like protein (CPLP), wherein the genome comprises a genetic modification such that the genetically-modified plant cell comprises the at least two copies of the CPLP gene. It was discovered herein that some genes within Vanilla sp. plants exhibit preferential allele expression. Thus, the two copies of the gene encoding a CPLP are alleles that actually express the CPLP polypeptide.

In certain embodiments, the CPLP gene and/or transcript comprises exons 1-3 (in some embodiments, the first three exons comprise the sequence set forth as amino acid residues 1-144 of SEQ ID NO: 9 or 11). In some embodiments, the CPLP polypeptide that is expressed from a specific CPLP allele capable of expression has the amino acid sequence set forth as SEQ ID NO: 9 or 11 or an active variant thereof (i.e., one that has CPLP enzymatic activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 9 or 11. In some of these embodiments, the active variant of SEQ ID NO: 3 retains amino acid residues 1-144 and/or a serine at a position corresponding to 151 of SEQ ID NO: 9 or 11.

In certain embodiments, the coding region of the CPLP allele capable of expression has the nucleotide sequence set forth as SEQ ID NO: 10 or 12 or an active variant thereof (i.e., one from which a polypeptide having CPLP enzymatic activity is actually expressed) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 10 or 12.

In some of these embodiments, the two copies of the CPLP gene can be within the endogenous genomic locus or within the genome, but outside of the naturally-occurring locus.

In certain embodiments, the genetically-modified plant cell, plant part, or plant comprises at least two copies of the CPLP gene within a chromosome pair (one copy on each chromosome of the pair) of a diploid genome. The two copies of the CPLP gene can be identical, making the genetically-modified plant cell, plant part, or plant homozygous for that particular CPLP gene sequence. In contrast, the two copies of the CPLP gene are not identical in sequence, making the genetically-modified plant cell, plant part, or plant heterozygous for that particular CPLP gene sequence.

Alternatively, the genetically-modified plant cell, plant part, or plant comprises at least two copies of the CPLP gene on a single chromosome.

The cell, plant part, or plant can be diploid or can exhibit polyploidy, which in some embodiments can be due to endoreduplication or partial endoreduplication. In the case of polyploidy, the genetically-modified plant cell, plant part, or plant can comprise two or more copies of a single chromosome or a part thereof and thus can comprise at least two copies of the CPLP gene with one or more copies on a single chromosome, or one or more copies on two or more chromosomes.

In some of these embodiments, the genetically-modified plant cell, plant part, or plant has increased levels of CPLP polypeptide and/or activity as compared to a non-genetically-modified plant cell, plant part, or plant. In some of these embodiments, the genetically-modified plant cell, plant part, or plant has at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more CPLP polypeptide levels and/or activity compared to a non-genetically-modified plant cell, plant part, or plant.

In certain embodiments, the genetically-modified plant cell, plant part, or plant produces increased levels of vanillin or one or more vanillin precursor, such as 4-hydroxybenzaldehyde, as compared to a non-genetically-modified plant cell, plant part, or plant. In some of these embodiments, the genetically-modified plant cell, plant part, or plant produces at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of vanillin or one or more vanillin precursor, such as 4-hydroxybenzaldehyde compared to a non-genetically-modified plant cell, plant part, or plant.

The genetically-modified plant cell can be within a plant part, such as a seed, seed capsule, or bean (comprising seeds and the seed capsule) or the genetically-modified plant part can be a genetically-modified seed, seed capsule, or bean. Genetically-modified seeds, seed capsules, or beans or an extract thereof can comprise increased levels of vanillin or one or more vanillin precursors, such as 4-hydroxybenzaldehyde, as compared to non-genetically-modified seeds, seed capsules, or beans or an extract thereof. In some of these embodiments, the genetically-modified seeds, seed capsules, or beans or an extract thereof have at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of vanillin or one or more vanillin precursor, such as 4-hydroxybenzaldehyde compared to a non-genetically-modified seed, seed capsule, or bean or extract thereof. In some of these embodiments wherein the genetically-modified seed, seed capsule, or bean comprises increased levels of vanillin or one or more vanillin precursors, the seed, seed capsule, or bean is green, has been freshly picked or has been cured on or off the plant as described elsewhere herein. Thus, the uncured genetically-modified vanilla seed, seed capsule or bean or an extract thereof can comprise increased levels of vanillin or one or more vanillin precursors or the curing process might be necessary for the genetically-modified vanilla seed, seed capsule, or bean or extract thereof to exhibit increased levels of vanillin or one or more vanillin precursors compared to a similarly processed control, non-genetically-modified vanilla seed, seed capsule, or bean or extract thereof.

Also described herein are genetically-modified plants or beans having at least one indehiscence-associated mutation in at least one dehiscent gene or reduced expression of at least one dehiscent gene compared to a non-genetically-modified plant or bean, wherein the genome comprises the at least one indehiscence-associated mutation in the at least one dehiscent gene or at least one genetic modification that reduces the expression of the at least one dehiscent gene compared to a non-genetically-modified plant or bean. In some embodiments, the dehiscent gene that is genetically-modified or reduced in expression encodes a Shatterproof, Indehiscent, Replumless, Adpg1, or Sh1 protein.

One or more copies of the dehiscent gene can be disrupted or knocked out or the promoter region can be mutated in order to reduce or inhibit expression. One or more nonsense mutations can be introduced into the dehiscent gene in order to encode a truncated protein that may or may not be functional or have reduced activity compared to the full-length protein.

In other embodiments, the genetically-modified Vanilla sp. plant or bean comprises at least one indehiscence-associated mutation in at least one dehiscent gene. The indehiscence-associated mutation can be a mutation from V. x tahitensis or another indehiscent Vanilla sp.

In some of those embodiments wherein the dehiscent gene that is mutated or for which the expression is reduced, the dehiscent gene is a gene encoding a Shatterproof protein. In certain embodiments, the Shatterproof protein has the sequence set forth as SEQ ID NO: 15 or an active variant thereof (i.e., one that has dehiscent activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 15. In those embodiments wherein an indehiscence-associated mutation is introduced into a Shatterproof protein, the mutation can be one that results in a leucine at a position corresponding to 149 of SEQ ID NO: 15, and/or a tyrosine at a position corresponding to 165 of SEQ ID NO: 15.

In other embodiments wherein the dehiscent gene that is mutated or for which the expression is reduced, the dehiscent gene is a gene encoding an Indehiscent protein. In certain embodiments, the Indehiscent protein has the sequence set forth as SEQ ID NO: 17 or an active variant thereof (i.e., one that has dehiscent activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 17. In those embodiments wherein an indehiscence-associated mutation is introduced into an Indehiscent protein, the mutation can be one that results in a serine inserted in between positions corresponding to 45 and 46 of SEQ ID NO: 17, and/or a proline at a position corresponding to 35 of SEQ ID NO: 17.

In still other embodiments wherein the dehiscent gene that is mutated or for which the expression is reduced, the dehiscent gene is a gene encoding a Replumless protein. In certain embodiments, the Replumless protein has the sequence set forth as SEQ ID NO: 19 or an active variant thereof (i.e., one that has dehiscent activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 19. In those embodiments wherein an indehiscence-associated mutation is introduced into a Replumless protein, the mutation can be one that results in a glycine at a position corresponding to 10 of SEQ ID NO: 19.

In yet other embodiments wherein the dehiscent gene that is mutated or for which the expression is reduced, the dehiscent gene is a gene encoding an ADPG1 protein. In certain embodiments, the ADPG1 protein has the sequence set forth as SEQ ID NO: 21 or an active variant thereof (i.e., one that has dehiscent activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 21. In those embodiments wherein an indehiscence-associated mutation is introduced into an ADPG1 protein, the mutation can be one that results in a tryptophan at a position corresponding to 29 of SEQ ID NO: 21, a serine at a position corresponding to 15 of SEQ ID NO: 21, and/or an aspartic acid at a position corresponding to 12 of SEQ ID NO: 21.

In particular embodiments wherein the dehiscent gene that is mutated or for which the expression is reduced, the dehiscent gene is a gene encoding a Sh1 protein. In certain embodiments, the Sh1 protein has the sequence set forth as SEQ ID NO: 23 or an active variant thereof (i.e., one that has dehiscent activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 23. In those embodiments wherein an indehiscence-associated mutation is introduced into a Sh1 protein, the mutation can be one that results in a threonine at a position corresponding to 113 of SEQ ID NO: 23.

The genetically-modified Vanilla sp. plant or bean having the one or more indehiscent mutations within one or more dehiscent genes or reduced expression of one or more dehiscent genes can exhibit reduced dehiscence compared to a non-genetically-modified Vanilla sp. plant or bean. Reduced dehiscence refers to a reduced dehiscence rate (at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or more reduction in the dehiscence rate) within a given population of beans of a single genetically-modified Vanilla sp. plant or a population of genetically-modified Vanilla sp. plants compared to a non-genetically-modified Vanilla sp. plant or a reduced probability (at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or more reduction in the probability of a single bean splitting) that a single genetically-modified Vanilla sp. bean will split compared to a non-genetically-modified Vanilla sp. bean.

Also described herein are genetically-modified Vanilla sp. plants having at least one mexicana-associated mutation in at least one MADS-box gene or reduced expression of at least one MADS-box gene compared to a non-genetically-modified Vanilla sp. plant, wherein the genome comprises the at least one mexicana-associated mutation in the at least one MADS-box gene or at least one genetic modification that reduces the expression of the at least one MADS-box gene compared to a non-genetically-modified Vanilla sp. plant.

One or more copies of the MADS-box gene(s) can be disrupted or knocked out or the promoter region can be mutated in order to reduce or inhibit expression. One or more nonsense mutations can be introduced into the MADS-box gene(s) in order to encode a truncated protein that may or may not be functional or have reduced activity compared to the full-length protein.

In other embodiments, the genetically-modified Vanilla sp. plant comprises at least one mexicana-associated mutation in at least one MADS-box gene.

In some of those embodiments wherein the MADS-box gene that is mutated or for which the expression is reduced, the MADS-box gene is a floral homeotic MADS-box gene. In some of these embodiments, the MADS-box gene is a C-class or D-class MADS-box gene. In particular embodiments, the MADS-box gene is an AGAMOUS or SEEDSTICK gene. In certain embodiments, the MADS-box gene encodes a MADS-box protein having the sequence of any one of SEQ ID NOs: 26, 28, 30, 32, and 34 or an active variant thereof (i.e., one that has floral (e.g., rostellum) development regulating activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to any one of SEQ ID NOs: 26, 28, 30, 32, and 34. In those embodiments wherein a mexicana-associated mutation is introduced into a MADS-box protein, the mutation can be determined by aligning the corresponding MADS-box protein from V. mexicana with any one of SEQ ID NOs: 26, 28, 30, 32, and 34 and introducing one or more of the amino acid residues from the V. mexicana MADS-box protein into SEQ ID NO: 26, 28, 30, 32, or 34.

In some of these embodiments, the genetically-modified Vanilla sp. plant has flowers that have a rostellum of reduced size compared to a non-genetically-modified Vanilla sp. plant or that lack a rostellum. The average size of the rostellum of flowers of the genetically-modified Vanilla sp. plant can be reduced by at least about 10%, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more compared to a non-genetically-modified Vanilla sp., plant. The reduction in size of the rostellum or absence of a rostellum can allow for self-pollination of the genetically-modified Vanilla sp. plant.

Also described herein are genetically-modified plant cells, plant parts, and plants having at least one pompona-associated mutation in at least one endogenous inactive fungal resistance gene such that the introduction of said at least one pompona-associated mutation in said endogenous inactive fungal resistance gene generates an active fungal resistance gene that encodes a fungal resistance protein, or a plant cell, plant part, or plant comprising at least one heterologous sequence encoding a fungal resistance protein.

In some embodiments, the fungal resistance protein can be a fungal resistance protein of V. pompona that imparts or contributes to fungal resistance or the endogenous inactive fungal resistance gene of a plant that has been genetically-modified to introduce mutations derived from V. pompona that impart activity to the encoded protein. In certain embodiments, the inactive fungal resistance protein has the sequence of any one of SEQ ID NOs: 36, 38 and 40 or an active variant thereof (i.e., one having fungal resistance activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to any one of SEQ ID NOs: 36, 38, and 40. In some of these embodiments, the inactive fungal resistance protein is mutated to comprise at least one of the amino acid residues selected from the group consisting of: a glycine, glutamic acid, histidine, glutamic acid, threonine, serine, lysine, histidine, leucine, isoleucine, glycine, arginine, leucine, aspartic acid, aspartic acid, glycine, asparagine, methionine, methionine, aspartic acid, glutamine, aspartic acid, asparagine, alanine, and glycine at positions corresponding to 28, 82, 91, 113, 131, 132, 147, 193, 199, 207, 227, 246, 271, 318, 324, 333, 336, 367, 379, 380, 408, 433, 443, 460, and 462, respectively of SEQ ID NO: 36; a glutamic acid, aspartic acid, glycine, methionine, methionine, threonine, isoleucine, lysine, arginine, lysine, asparagine, phenylalanine, lysine, proline, phenylalanine, lysine, and alanine at positions corresponding to 29, 107, 216, 229, 362, 404, 547, 574, 610, 638, 695, 706, 773, 840, 860, 870, and 889, respectively of SEQ ID NO: 38; and an alanine, glycine, isoleucine, glutamic acid, alanine, serine, tyrosine, methionine, lysine, glutamine, lysine, and serine at positions corresponding to 91, 124, 227, 333, 381, 537, 555, 703, 716, 754, 758, and 768, respectively of SEQ ID NO: 40

In some of these embodiments, the genetically-modified plant cell, plant part, or plant or plant cell, plant part, or plant having the heterologous sequence encoding a fungal resistance protein has increased resistance to a fungus compared to a non-genetically-modified plant cell, plant part, or plant or a plant cell, plant part, or plant lacking a heterologous sequence encoding a fungal resistance protein. In particular embodiments, the genetically-modified plant cell, plant part, or plant or plant cell, plant part, or plant having the heterologous sequence encoding a fungal resistance protein has an increase of at least about 10%, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more of resistance activity against a fungus compared to a non-genetically-modified plant cell, plant part, or plant or a plant cell, plant part, or plant lacking a heterologous sequence encoding a fungal resistance protein. In some of these embodiments, the genetically-modified plant cell, plant part, or plant or plant cell, plant part, or plant having the heterologous sequence encoding a fungal resistance protein has a reduction of symptoms associated with a fungal infection or a disease resulting therefrom of at least about 10%, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more compared to a non-genetically-modified plant cell, plant part, or plant or a plant cell, plant part, or plant lacking a heterologous sequence encoding a fungal resistance protein.

While the genetically-modified plant cell, plant part, or plant or plant cell, plant part, or plant having the heterologous sequence encoding a fungal resistance protein can exhibit resistance to any type of fungus, in some embodiments, the fungus is a Fusarium sp., including but not limited to, F. oxysporum f. sp. vanilla.

In any one of the embodiments disclosed herein, the genetically-modified plant part or plant part comprising a heterologous sequence is a seed, a seed capsule, or a bean. Extracts of the plant, seed, seed capsule, or bean (uncured or cured) are also provided herein.

### 2.3. Methods for Genetically-Modifying Plant Cells and Producing Plants

Described herein are methods for genetically-modifying or introducing heterologous sequences into plants, plant parts, or plant cells and producing plants by growing a plant from the genetically-modified plant parts or plant cells or plant parts or plant cells with a heterologous sequence.

Methods comprise introducing at least one copy of a gene encoding a PAL into the genome of a plant cell by genetically-modifying the genome of the plant cell to comprise at least two copies of the PAL gene to generate a genetically-modified plant cell. Additional methods comprise producing a plant having at least two copies of a gene encoding a PAL by genetically-modifying the genome of a plant cell or plant part to comprise at least two copies of the PAL gene to generate a genetically-modified plant cell or plant part, and growing a plant from the genetically-modified plant cell or plant part, wherein the plant has at least two copies of the PAL gene.

The at least one copy of a PAL gene can be introduced by genetically modifying an endogenous sequence that has homology with a PAL gene to introduce mutations. This endogenous sequence could be present within the plant cell, part, or plant due to endoreduplication of the genome, but could be inactive or unable to encode an active PAL protein. Genetic modification to introduce various mutations (for example, missense mutations that result in an amino acid substitution to include amino acid residues that are present in an active PAL gene, such as one or more of the amino acid residues that are present in SEQ ID NO: 1, but not SEQ ID NO: 3, 5, and 7, as shown in the alignment provided in FIG. 16) into the endogenous sequence may be sufficient to convert the inactive gene into an active gene encoding an active PAL protein. Alternatively, the genome can be genetically-modified to introduce a full-length PAL gene or a part thereof to complement an endogenous sequence and allow for the encoding of a full-length PAL protein. These larger sequences can be introduced via a vector or expression cassette, further comprising regulatory sequences (e.g., promoter) to allow for expression of the newly introduced PAL gene.

In some embodiments, the PAL polypeptide that is expressed from the introduced PAL gene has the amino acid sequence set forth as SEQ ID NO: 1 or an active variant thereof (i.e., one that has PAL enzymatic activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 1. In some of these embodiments, the active variant of SEQ ID NO: 1 retains one or more of the amino acid residues that are present in SEQ ID NO: 1, but not SEQ ID NO: 3, 5, and 7, as shown in the alignment provided in FIG. 16.

In certain embodiments, the PAL gene that is introduced into the plant cell or plant part has the nucleotide sequence set forth as SEQ ID NO: 2 or an active variant thereof (i.e., one from which a polypeptide having PAL enzymatic activity is actually expressed) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 2.

In some embodiments, the plant cell or plant part into which at least one copy of a PAL gene is introduced already comprises one copy of a PAL gene. In some of these embodiments, only one PAL gene is introduced to bring the total copy number of the cell or plant part to two copies.

In certain embodiments, the at least one copy of the PAL gene is introduced into the endogenous genomic locus or within the genome, but outside of the naturally-occurring locus.

In certain embodiments, the at least one copy of the PAL gene is introduced such that the genetically-modified plant cell, plant part, or plant comprises at least two copies of the PAL gene within a chromosome pair (one copy on each chromosome of the pair) of a diploid genome. The two copies of the PAL gene can be identical, making the genetically-modified plant cell, plant part, or plant homozygous for that particular PAL gene sequence.

In contrast, the two copies of the PAL gene are not identical in sequence, making the genetically-modified plant cell, plant part, or plant heterozygous for that particular PAL gene sequence.

Alternatively, the at least one copy of the PAL gene is introduced such that the genetically-modified plant cell, plant part, or plant comprises at least two copies of the PAL gene on a single chromosome.

The cell, plant part, or plant can be diploid or can exhibit polyploidy, which in some embodiments can be due to endoreduplication or partial endoreduplication. In the case of polyploidy, the genetically-modified plant cell, plant part, or plant can comprise two or more copies of a single chromosome or a part thereof and thus can comprise at least two copies of the PAL gene with one or more copies on a single chromosome, or one or more copies on two or more chromosomes.

In some of these embodiments, the genetically-modified plant cell, plant part, or plant has increased levels of PAL polypeptide and/or activity as compared to a non-genetically-modified plant cell, plant part, or plant. In some of these embodiments, the genetically-modified plant cell, plant part, or plant has at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more PAL polypeptide levels and/or activity compared to a non-genetically-modified plant cell, plant part, or plant.

In certain embodiments, the genetically-modified plant cell, plant part, or plant produces increased levels of vanillin or one or more vanillin precursor, such as cinnamic acid (e.g., trans-cinnamic acid), as compared to a non-genetically-modified plant cell, plant part, or plant. In some of these embodiments, the genetically-modified plant cell, plant part, or plant produces at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of vanillin or one or more vanillin precursor, such as cinnamic acid (e.g., trans-cinnamic acid) compared to a non-genetically-modified plant cell, plant part, or plant.

The genetically-modified plant cell can be within a plant part, such as a seed, seed capsule, or bean (comprising seeds and the seed capsule) or the genetically-modified plant part can be a genetically-modified seed, seed capsule, or bean. Genetically-modified seeds, seed capsules, or beans or an extract thereof can comprise increased levels of vanillin or one or more vanillin precursors, such as cinnamic acid (e.g., trans-cinnamic acid), as compared to non-genetically-modified seeds, seed capsules, or beans or an extract thereof. In some of these embodiments, the genetically-modified seeds, seed capsules, or beans or an extract thereof have at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of vanillin or one or more vanillin precursor, such as cinnamic acid (e.g., trans-cinnamic acid) compared to a non-genetically-modified seed, seed capsule, or bean or extract thereof. In some of these embodiments wherein the genetically-modified seed, seed capsule, or bean comprises increased levels of vanillin or one or more vanillin precursors, the seed, seed capsule, or bean is green, has been freshly picked or has been cured on or off the plant as described elsewhere herein. Thus, the uncured genetically-modified vanilla seed, seed capsule or bean or an extract thereof can comprise increased levels of vanillin or one or more vanillin precursors or the curing process might be necessary for the genetically-modified vanilla seed, seed capsule, or bean or extract thereof to exhibit increased levels of vanillin or one or more vanillin precursors compared to a similarly processed control, non-genetically-modified vanilla seed, seed capsule, or bean or extract thereof.

Methods comprise introducing at least one copy of a gene encoding a CPLP into the genome of a plant cell by genetically-modifying the genome of the plant cell to comprise at least two copies of the CPLP gene to generate a genetically-modified plant cell. Additional methods comprise producing a plant having at least two copies of a gene encoding a CPLP by genetically-modifying the genome of a plant cell or plant part to comprise at least two copies of the CPLP gene to generate a genetically-modified plant cell or plant part, and growing a plant from the genetically-modified plant cell or plant part, wherein the plant has at least two copies of the CPLP gene.

The at least one copy of a CPLP gene can be introduced by genetically modifying an endogenous sequence that has homology with a CPLP gene to introduce mutations. This endogenous sequence could be present within the plant cell, part, or plant due to endoreduplication of the genome, but could be inactive or unable to encode an active CPLP protein.

Genetic modifications to introduce various mutations (for example, missense mutations that result in an amino acid substitution to include amino acid residues that are present in an active CPLP gene, such as amino acid residues 1-144 and/or a serine at a position corresponding to 151 of SEQ ID NO: 9 or 11) into the endogenous sequence may be sufficient to convert the inactive gene into an active gene encoding an active CPLP protein. Alternatively, the genome can be genetically-modified to introduce a full-length CPLP gene or a part thereof to complement an endogenous sequence and allow for the encoding of a full-length CPLP protein. In some of these embodiments, the genome can be genetically-modified to add exons 1-3 to the amino terminus of the encoded protein. These larger sequences can be introduced via a vector or expression cassette, further comprising regulatory sequences (e.g., promoter) to allow for expression of the newly introduced CPLP gene.

In some embodiments, the CPLP polypeptide that is expressed by the introduced or genetically-modified CPLP gene has the amino acid sequence set forth as SEQ ID NO: 9 or 11 or an active variant thereof (i.e., one that has CPLP enzymatic activity) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 9 or 11. In some of these embodiments, the active variant of SEQ ID NO: 3 retains amino acid residues 1-144 and/or a serine at a position corresponding to 151 of SEQ ID NO: 9 or 11.

In certain embodiments, the introduced CPLP gene has the nucleotide sequence set forth as SEQ ID NO: 10 or 12 or an active variant thereof (i.e., one from which a polypeptide having CPLP enzymatic activity is actually expressed) having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to SEQ ID NO: 10 or 12.

In some embodiments, the plant cell or plant part into which at least one copy of a CPLP gene is introduced already comprises one copy of a CPLP gene. In some of these embodiments, only one CPLP gene is introduced to bring the total copy number of the cell or plant part to two copies.

In certain embodiments, the at least one copy of the CPLP gene is introduced into the endogenous genomic locus or within the genome, but outside of the naturally-occurring locus.

In certain embodiments, the at least one copy of the CPLP gene is introduced such that the genetically-modified plant cell, plant part, or plant comprises at least two copies of the CPLP gene within a chromosome pair (one copy on each chromosome of the pair) of a diploid genome. The two copies of the CPLP gene can be identical, making the genetically-modified plant cell, plant part, or plant homozygous for that particular CPLP gene sequence. In contrast, the two copies of the CPLP gene are not identical in sequence, making the genetically-modified plant cell, plant part, or plant heterozygous for that particular CPLP gene sequence.

Alternatively, the at least one copy of the CPLP gene is introduced such that the genetically-modified plant cell, plant part, or plant comprises at least two copies of the CPLP gene on a single chromosome.

The cell, plant part, or plant can be diploid or can exhibit polyploidy, which in some embodiments can be due to endoreduplication or partial endoreduplication. In the case of polyploidy, the genetically-modified plant cell, plant part, or plant can comprise two or more copies of a single chromosome or a part thereof and thus can comprise at least two copies of the CPLP gene with one or more copies on a single chromosome, or one or more copies on two or more chromosomes.

In some of these embodiments, the genetically-modified plant cell, plant part, or plant has increased levels of CPLP polypeptide and/or activity as compared to a non-genetically-modified plant cell, plant part, or plant. In some of these embodiments, the genetically-modified plant cell, plant part, or plant has at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more CPLP polypeptide levels and/or activity compared to a non-genetically-modified plant cell, plant part, or plant.

In certain embodiments, the genetically-modified plant cell, plant part, or plant produces increased levels of vanillin or one or more vanillin precursor, such as 4-hydroxybenzaldehyde, as compared to a non-genetically-modified plant cell, plant part, or plant. In some of these embodiments, the genetically-modified plant cell, plant part, or plant produces at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of vanillin or one or more vanillin precursor, such as 4-hydroxybenzaldehyde compared to a non-genetically-modified plant cell, plant part, or plant.

The genetically-modified plant cell can be within a plant part, such as a seed, seed capsule, or bean (comprising seeds and the seed capsule) or the genetically-modified plant part can be a genetically-modified seed, seed capsule, or bean. Genetically-modified seeds, seed capsules, or beans or an extract thereof can comprise increased levels of vanillin or one or more vanillin precursors, such as 4-hydroxybenzaldehyde, as compared to non-genetically-modified seeds, seed capsules, or beans or an extract thereof. In some of these embodiments, the genetically-modified seeds, seed capsules, or beans or an extract thereof have at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of vanillin or one or more vanillin precursor, such as 4-hydroxybenzaldehyde compared to a non-genetically-modified seed, seed capsule, or bean or extract thereof. In some of these embodiments wherein the genetically-modified seed, seed capsule, or bean comprises increased levels of vanillin or one or more vanillin precursors, the seed, seed capsule, or bean is green, has been freshly picked or has been cured on or off the plant as described elsewhere herein. Thus, the uncured genetically-modified vanilla seed, seed capsule or bean or an extract thereof can comprise increased levels of vanillin or one or more vanillin precursors or the curing process might be necessary for the genetically-modified vanilla seed, seed capsule, or bean or extract thereof to exhibit increased levels of vanillin or one or more vanillin precursors compared to a similarly processed control, non-genetically-modified vanilla seed, seed capsule, or bean or extract thereof.

Provided herein are also methods comprising introducing at least one indehiscence-associated mutation into at least one dehiscent gene or reducing the expression of at least one dehiscent gene in a Vanilla sp. plant or bean. Additional methods comprise producing a Vanilla sp. plant having at least one indehiscence-associated mutation into at least one dehiscent gene or reduced expression of at least one dehiscent gene by introducing at least one indehiscence-associated mutation into at least one dehiscent gene or reducing the expression of at least one dehiscent gene in a Vanilla sp. plant cell or plant part, and then growing a plant from the genetically-modified plant cell or plant part.

In some embodiments, the dehiscent gene that is genetically-modified or reduced in expression encodes a Shatterproof, Indehiscent, Replumless, Adpg1, or Sh1 protein.

One or more copies of the dehiscent gene can be disrupted or knocked out or the promoter region can be mutated in order to reduce or inhibit expression. One or more nonsense mutations can be introduced into the dehiscent gene in order to encode a truncated protein that may or may not be functional or have reduced activity compared to the full-length protein. The promoter region of a dehiscent gene can be mutated to reduce the expression of the gene. Alternatively, the expression of a gene can be reduced through post-transcriptional gene silencing that utilizes the RNA interference pathway, such as introducing and/or expressing siRNAs, shRNAs, or miRNAs.

In other embodiments, the genetically-modified Vanilla sp. plant or bean comprises at least one indehiscence-associated mutation in at least one dehiscent gene. The indehiscence-associated mutation can be a mutation from V. x tahitensis or another indehiscent Vanilla sp.

Also provided herein are methods for producing a Vanilla sp. plant having at least one mexicana-associated mutation in at least one MADS-box gene or reduced expression of at least one MADS-box gene by genetically-modifying the genome of a Vanilla sp. plant cell or plant part to introduce at least one mexicana-associated mutation into at least one MADS-box gene or to reduce the expression of at least one MADS-box gene, and then growing a plant from the genetically-modified Vanilla sp. plant cell or plant part.

One or more copies of the MADS-box gene(s) can be disrupted or knocked out or the promoter region can be mutated in order to reduce or inhibit expression. One or more nonsense mutations can be introduced into the MADS-box gene(s) in order to encode a truncated protein that may or may not be functional or have reduced activity compared to the full-length protein. The promoter region of a MADS-box gene can be mutated to reduce the expression of the gene.

In other embodiments, the genetically-modified Vanilla sp. plant comprises at least one mexicana-associated mutation in at least one MADS-box gene.

Provided herein are methods comprising introducing into a plant cell at least one heterologous sequence encoding a fungal resistance protein or genetically-modifying the genome of a plant cell to introduce at least one pompona-associated mutation into at least one endogenous inactive fungal resistance gene that generates an active fungal resistance gene that encodes a fungal resistance protein. Additional methods comprise producing a plant having at least one heterologous sequence encoding a fungal resistance protein or genetically-modifying the genome of a plant cell or plant part to introduce at least one pompona-associated mutation within at least one endogenous inactive fungal resistance gene that generates an active fungal resistance gene, and then growing a plant from the genetically-modified plant cell or plant part or the plant cell or plant part comprising the heterologous sequence.

The heterologous sequence can be introduced as an expression cassette further comprising a promoter operably linked to the sequence encoding the fungal resistance protein.

Any method known in the art can be used to introduce a heterologous sequence or to introduce mutations.

By “introducing” is intended to introduce the nucleotide construct to the plant or other host cell in such a manner that the construct gains access to the interior of a cell of the plant or host cell. The methods of the present disclosure do not require a particular method for introducing a nucleotide construct to a plant or host cell, only that the nucleotide construct gains access to the interior of at least one cell of the plant or the host organism.

Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen).

As used herein, the term “transgenic” or “transformed” or “stably transformed” plants or cells or tissues refers to plants or cells or tissues that have been modified by the methods of the present disclosure. In contrast, control, non-transgenic, or unmodified plants or cells or tissues refer to plants or cells or tissues that are without such modifications. It is recognized that other exogenous or endogenous nucleic acid sequences or DNA fragments may also be incorporated into the plant cell. Agrobacterium- and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, Agro and viral mediated (Caulimoriviruses, Geminiviruses, RNA plant viruses), liposome mediated and the like.

Transformation protocols as well as protocols for introducing polypeptides or polynucleotide sequences into plants may vary depending on the type of plant or plant cell, i.e., monocot or dicot, targeted for transformation. Methods for transformation are known in the art and include those set forth in U.S. Pat. Nos. 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

Although stable transformation is presently preferred, transient transformation of leaf cells, meristematic cells or the whole plant is also envisaged by some embodiments of the disclosure.

Transient transformation can be effected by any of the direct DNA transfer methods described above or by viral infection using modified plant viruses.

Viruses that have been shown to be useful for the transformation of plant hosts include CaMV, TMV, and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is used in the presently disclosed methods in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced.

The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In another embodiment, a recombinant plant viral nucleic acid is provided as in the first embodiment except that the native coat protein coding sequence is placed adjacent one of the non-native coat protein subgenomic promoters instead of a non-native coat protein coding sequence.

In a different embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In an alternative embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In some embodiments, the heterologous sequence is introduced as a vector or an expression construct. In some embodiments, the expression construct contains a cis-acting regulatory element that is operably linked to the one or more coding sequence. In particular embodiments, the cis-acting regulatory element is a promoter.

In some embodiments, an expression construct used in the presently disclosed methods may contain a promoter sequence, a leader sequence, and/or one or more nuclease recognition sites. In certain embodiments, an expression construct disclosed herein may be a repair template, such as a repair template containing a promoter (e.g., a viral promoter, such as a CsVMV promoter) and a leader sequence (e.g., a SynJ 5′ leader sequence) inserted between a pair of nuclease recognition sites (e.g., a pair of engineered meganuclease cleavage sites).

In some embodiments, an expression construct used in the presently disclosed methods may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the present disclosure. The promoters can be selected based on the desired outcome. The nucleic acids can be combined with constitutive, inducible, tissue-preferred, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730).

Tissue-preferred promoters for use in the invention include those set forth in Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505.

Leaf-preferred promoters include those set forth in Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and include those in Hire et al. (1992) Plant Mol. Biol. 20(2):207-218 (soybean root-specific glutamine synthetase gene); Keller and Baumgartner (1991) Plant Cell 3(10):1051-1061 (root-specific control element); Sanger et al. (1990) Plant Mol. Biol. 14(3):433-443 (mannopine synthase (MAS) gene of Agrobacterium tumefaciens); and Miao et al. (1991) Plant Cell 3(1):11-22 (cytosolic glutamine synthetase (GS)); Bogusz et al. (1990) Plant Cell 2(7):633-641; Leach and Aoyagi (1991) Plant Science (Limerick) 79(1):69-76 (rolC and rolD); Teeri et al. (1989) EMBO J. 8(2):343-350; Kuster et al. (1995) Plant Mol. Biol. 29(4):759-772 (the VfENOD-GRP3 gene promoter); and, Capana et al. (1994) Plant Mol. Biol. 25(4):681-691 (rolB promoter). See also U.S. Pat. Nos. 5,837,876; 5,750,386; 5,633,363; 5,459,252; 5,401,836; 5,110,732; and U.S. Pat. No. 5,023,179.

“Seed-preferred” promoters include both “seed-specific” promoters (those promoters active during seed development such as promoters of seed storage proteins) as well as “seed-germinating”promoters (those promoters active during seed germination). See Thompson et al. (1989) BioEssays 10:108. Seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529). Gamma-zein is an endosperm-specific promoter. Globulin 1 (Glb-1) is a representative embryo-specific promoter. For dicots, seed-specific promoters include, but are not limited to, bean 0-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed.

In specific embodiments, the sequences provide herein can be targeted to specific site within the genome of the host cell or plant cell or specific mutations at particular sites are introduced. Methods for targeting sequence to specific sites in the genome can include the use of engineered nucleases.

It is known in the art that it is possible to use a site-specific nuclease to make a DNA break in the genome of a living cell, and that such a DNA break can result in permanent modification of the genome via homologous recombination with a transgenic DNA sequence. The use of nucleases to induce a double-strand break in a target locus is known to stimulate homologous recombination, particularly of transgenic DNA sequences flanked by sequences that are homologous to the genomic target. In this manner, exogenous nucleic acids can be inserted into a target locus.

It is known in the art that it is possible to use a site-specific nuclease to make a DNA break in the genome of a living cell, and that such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. Further, the use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus.

Thus, in different embodiments, a variety of different types of nucleases are useful for practicing the invention. In one embodiment, the invention can be practiced using engineered recombinant meganucleases. In another embodiment, the invention can be practiced using a CRISPR system nuclease (e.g., CRISPR/Cas9 or RNA-guided nucleases such as Cpf1, MAD7, etc.), or CRISPR system nickase. Methods for making CRISPR and CRISPR Nickase systems that recognize and bind pre-determined DNA sites are known in the art, for example Ran, et al. (2013) Nat Protoc. 8:2281-308. In another embodiment, the invention can be practiced using TALENs or Compact TALENs. Methods for making TALE domains that bind to pre-determined DNA sites are known in the art, for example Reyon et al. (2012) Nat Biotechnol. 30:460-5. In another embodiment, the invention can be practiced using zinc finger nucleases (ZFNs). In a further embodiment, the invention can be practiced using megaTALs.

In some embodiments, the nucleases used to practice the invention are meganucleases. In particular embodiments, the nucleases used to practice the invention are single-chain meganucleases. A single-chain meganuclease comprises an N-terminal subunit and a C-terminal subunit joined by a linker peptide. Each of the two domains recognizes and binds to half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs.

In some embodiments, systems used to edit the genomes of plants include but are not limited to, engineered meganucleases (e.g., homing endonucleases) designed against the plant genomic sequence of interest (D'Halluin et al. 2013 Plant Biotechnol J); CRISPR-Cas9, alternative CRISPR editing systems known in the art (e.g., RNA-guided nucleases, such as Cpf1, MAD7, etc.), TALENs, and other technologies can be used for precise editing of genomes (e.g., Feng, et al. Cell Research 23:1229-1232, 2013, Podevin, et al. Trends Biotechnology, online publication, 2013, Wei et al., J Gen Genomics, 2013, Zhang et al (2013) WO 2013/026740); Cre-lox site-specific recombination (Dale et al. (1995) Plant J 7:649-659; Lyznik, et al. (2007) Transgenic Plant J 1:1-9; FLP-FRT recombination (Li et al. (2009) Plant Physiol 151:1087-1095); Bxb1-mediated integration (Yau et al. Plant J (2011) 701:147-166); zinc-finger nuclease mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol 701: 51-65); Puchta (2002) Plant Mol Biol 48:173-182).

### 2.4. Methods of Creating a Population of Plants

Methods are provided for creating a population of Vanilla sp. plants having at least two copies of a gene encoding a PAL or CPLP, at least one indehiscence-associated mutation in at least one dehiscent gene, at least one Mexicana-associated mutation in at least one MADS-box gene, at least one pompona-associated mutation within at least one endogenous inactive fungal resistance gene, or a heterologous sequence encoding a fungal resistance gene by detecting the presence of the two copies of a gene encoding a PAL or CPLP, at least one indehiscence-associated mutation in at least one dehiscent gene, at least one Mexicana-associated mutation in at least one MADS-box gene, at least one pompona-associated mutation within at least one endogenous inactive fungal resistance gene, or the heterologous sequence in a DNA sample from at least one plant within a first population of Vanilla sp. plants, selecting one or more Vanilla sp. plants from the first population based on the presence of the two copies of a gene encoding a PAL or CPLP, at least one indehiscence-associated mutation in at least one dehiscent gene, at least one Mexicana-associated mutation in at least one MADS-box gene, at least one pompona-associated mutation within at least one endogenous inactive fungal resistance gene, or the heterologous sequence in the DNA sample, and then crossing the selected Vanilla sp. plant with itself or another, different Vanilla sp. plant to produce a population of offspring. The offspring population comprises the two copies of a gene encoding a PAL or CPLP, at least one indehiscence-associated mutation in at least one dehiscent gene, at least one Mexicana-associated mutation in at least one MADS-box gene, at least one pompona-associated mutation within at least one endogenous inactive fungal resistance gene, or the heterologous sequence.

Any method known in the art for generating the DNA sample can be used in the presently disclosed methods. The DNA sample can be derived from an entire plant or a part thereof.

Any method known in the art to detect the heterologous sequence, the mutations, or the two gene copies, including but not limited to, polymerase chain reaction, Southern blotting, or sequencing-based methods.

Any method known in the art can be used to cross the selected plant with itself or with another plant, including traditional or new breeding technologies (Chambers, Advances in Plant Breeding Strategies: Industrial and Food Crops, Ch. 18 (Springer, 2019), which is herein incorporated in its entirety).

Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting.

### EXAMPLES

The Examples below are merely illustrative, and are not intended to limit the scope of the disclosure provided herein in any way.

### Example 1. A Chromosome-Scale Assembly of the Vanilla planifolia Genome to Expedite Genetic Improvement of Flavor and Agronomic Production

The following example describes a chromosome-scale, phased genome sequence of V. planifolia that reveals haplotype-specific sequence and transcript abundance differences within the commercially-relevant vanillin pathway that impacts bean quality. Resequencing of related vanilla species identified genes that could impact productivity and post-harvest losses through pod dehiscence, flower anatomy, and disease resistance.

### 1.1 Methods

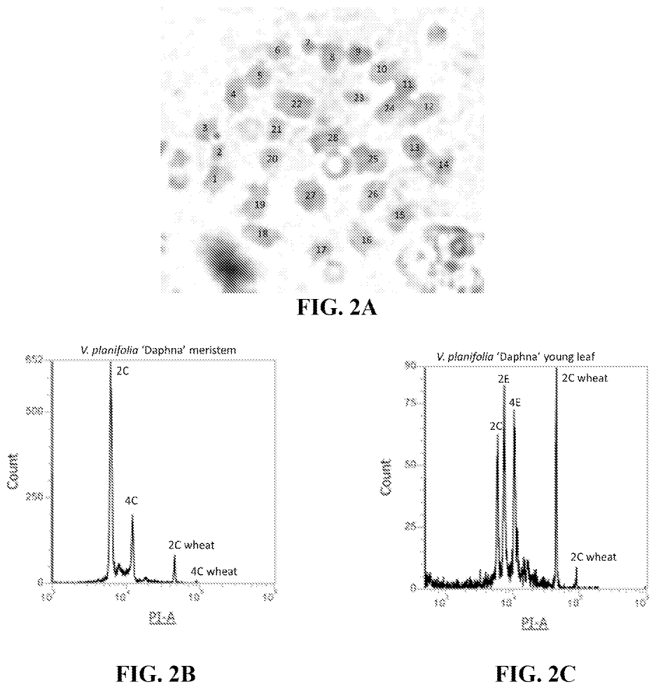

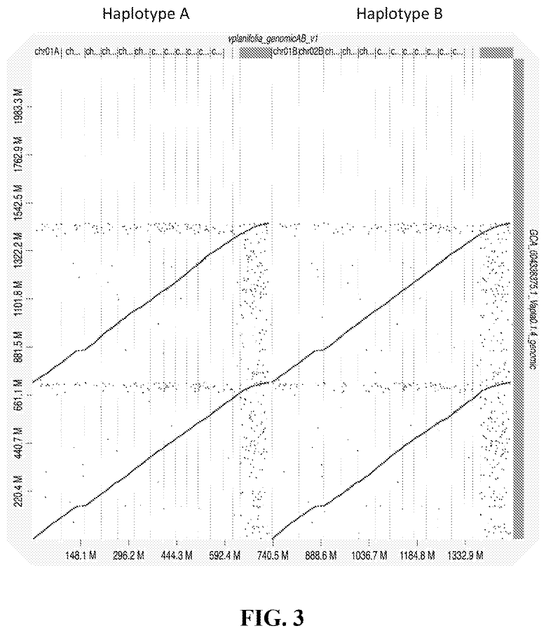

Flow Cytometry and Chromosome Counts Orchid leaves commonly exhibit partial endoreduplication confounding genome size estimation (Brown et al. (2017) Genome Biol. Evol. 9:1051-1071). Therefore, flow cytometry was performed on vanilla apical meristematic tips as this specific tissue has a higher proportion of non-endoreduplicated nuclei than other tissues (FIG. 2 B, C) (Brown et al. (2017)). For analysis, 2-3 mm apical tips without green tissue were finely chopped in a petri dish on ice with 0.5 cm×0.5 cm pea or wheat leaf tissue using a fresh razor blade in 1.5 ml GPB buffer (Loureiro et al. (2007) Ann. Bot. 100:875-888), filtered through a 20 m nylon mesh (Partec, CellTrics), and analyzed using an Attune NxT flow cytometer (ThermoScientific) equipped with a blue excitation laser (BL 275 volts, SSC 300 V, FSC 300 V). Wheat (2C=31.9 pg) and pea (2C=9.07 pg) were used in 2C calculations as previously reported (Dolezel and Bartos (2005) Ann. Bot. 95:99-110). P was calculated as the relative fluorescence intensity peak of the 2C nuclei population divided by the relative fluorescence intensity of the first endoreduplicated nuclei population as previously described (Brown et al. (2017)).

A modified chromosome staining protocol was adapted from previous work (Aliyeva-Schnorr et al. (2015) JoVE J. Vis. Exp. Doi:10.3791/53470; Kirov et al. (2014) Molec. Cytogenet. 7:21). A 5-10 mm section of apical meristem without green tissue was harvested, sliced into 1 mm strips lengthwise, and placed in 1 ml 0.5% colchicine for 24 hours on a rotating shaker at 21° C. in the dark. The meristem was then fixed in 1 ml 3:1 absolute ethanol:glacial acetic acid (v/v) for 16-24 hours at 21° C. in the dark. The meristem tissue was then digested in 1 ml of an enzyme mixture (1% cellulase, 0.5% pectolyase, and 1% hemicellulase) of 75 mM KCl for 90 minutes at 32° C. Tissue was broken up by pipetting with a cut-off 1 ml pipette tip and centrifuged for 5 minutes at 200 x g. The enzyme solution was aspirated, and the pellet was gently washed successively with 75 mM KCl then 95% EtOH with centrifugation after each step as before. The pellet was finally resuspended in 50 μl of 95% EtOH. 10 μl of the cell suspension was pipetted from 20 cm above an ice-cold slide placed on a moist paper towel on a 55° C. hot plate and allowed to dry for ˜15 seconds. Then 10 μl 3:1 absolute ethanol:glacial acetic acid (v/v) was pipetted onto the slide from the same distance and left for 2 minutes. The slide was then removed from the paper towels and incubated on the hot plate for 1 min. For staining, 15 μl of 2% aceto-orcein stain was placed on top of the chromosome spread followed by a cover slip. The chromosomes were viewed using an oil immersion lens at 100× magnification.

### Sequencing and Resequencing

High-molecular weight DNA from V. planifolia ‘Daphna’ was extracted by KeyGene N.V. (Wageningen, Netherlands) using nuclei isolated from frozen leaves ground under liquid nitrogen as previously reported (Zhang et al. (2012) Nat. Protoc. 7:467-478; Datema et al. (2016) “The megabase-sized fungal genome of Rhizoctonia solani assembled from nanopore reads only” bioRxiv). Leaf tissue from orchids commonly show partial endoreduplication being a phenomenon where multiple genomes are present within a nucleus. Multiple genome copies per cell are not anticipated to significantly impact genome assembly as the pool of DNA would be similar to that from many individual plant cells or when sequencing stable polyploids. DNA extractions for resequencing additional accessions were performed using a modified CTAB method as previously described (Hu et al. (2019) Sci. Rep. 9:3416). DNA library preparation and sequencing of the V. planifolia genome were executed by KeyGene and QuickBiology (Pasadena, CA) using long- and short-read platforms. The long-read sequencing was performed with the GridION and PromethION sequencers from Oxford Nanopore Techonologies (ONT). The 1D genomic libraries were constructed with the ligation sequencing kit SQK-LSK109 (ONT). Two GridION cells were used for initial QC. Subsequently, a total of six PromethION FLO-PRO002 (R9.4.1 pore) cells were used to generate the data. Basecalling was performed real-time on the compute module (PromethION release: 18.07.1-3-xenial and MinKNOW 1.14.2). The short-read paired-end sequencing (2×150 bp) was conducted on two lanes of an Illumina HiSeq4000 System from PCR-free WGS libraries with an insert size of 550 bp. The resequencing of additional vanilla accessions was also performed by GENEWIZ using the same short-read approach described above.

### Chromosome Conformation

Chromosome conformation libraries were prepared by Dovetail Genomics (Scotts Valley, California) using the Dovetail Hi-C Kit and following the recommended protocol (Lieberman-Aiden et al. (2009) Science 326:289-293). Briefly, the intact cells from leaf samples were crosslinked using a formaldehyde solution, digested using the DpnII restriction enzyme, and proximity ligated with biotinylated nucleotides to create chimeric molecules composed of fragments from different regions of the genome that were physically proximal in vivo. The molecules were pulled down with streptavidin beads and processed into an Illumina-compatible sequencing library that was sequenced using paired end reads (2×150 bp).

### Assembly

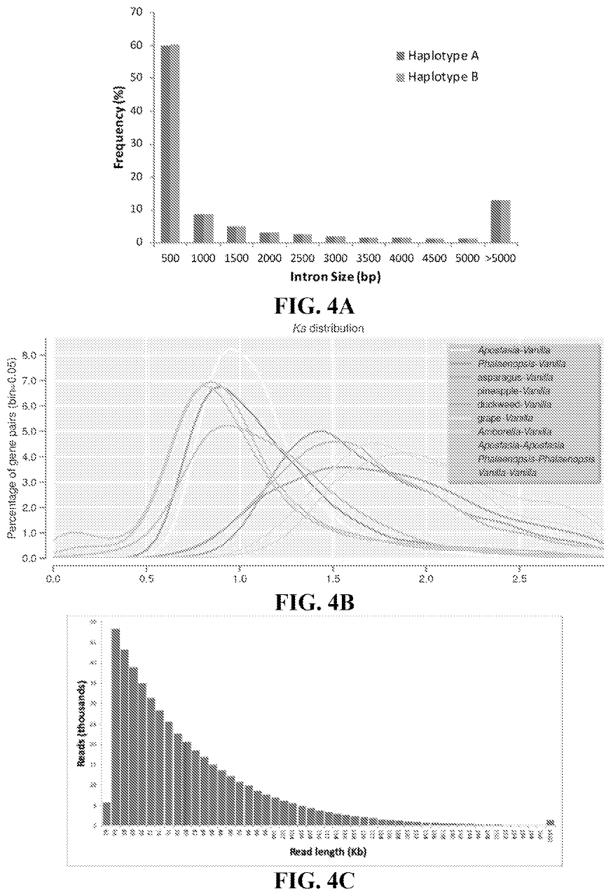

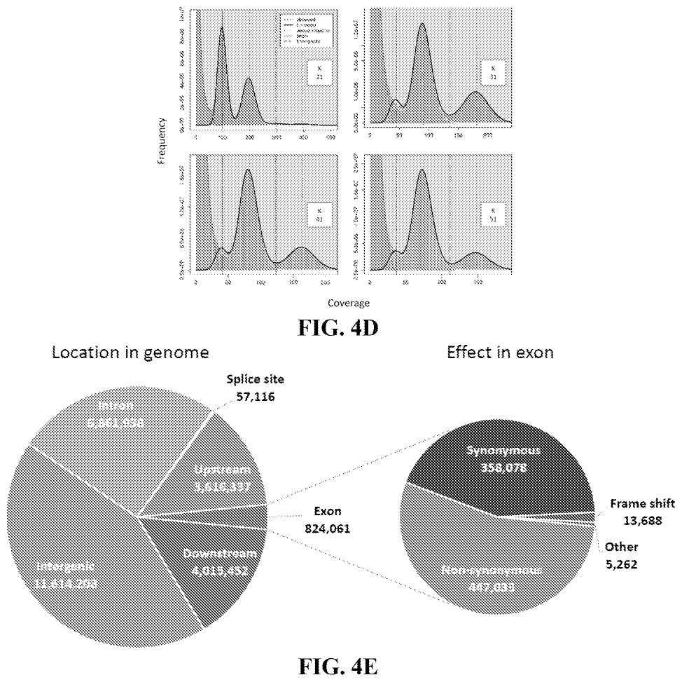

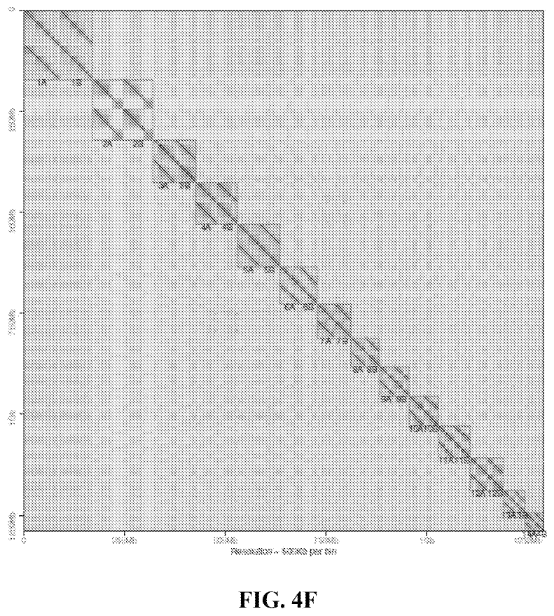

The ‘Daphna’ draft genome was assembled from ONT long reads and Illumina short reads. ONT reads were filtered to include only those greater than ˜60 kbp (FIG. 4C). This subset of 472,075 reads totaled 37.5 Gb and represents a coverage of 18×, 50×, or 51-59× as estimated by 1C genome sizes from flow cytometry (1C˜2.1 Gb), the genome assembly (1C˜0.740 Gb), or by kmer analysis (1C˜0.64-0.73 Gb), respectively. The overlaps between the long reads were identified with Minimap2 v2,11-r797 (parameter-x ava-ont) (Li (2018) Bioinf 34:3094-3100), which were then used to construct an initial assembly using Miniasm v0.3-r179 (default parameters) (Li (2016) Bioinf 32:2103-2110). The base accuracy of the initial assembly was further improved through consensus generation using the same ONT reads with Racon v1.4.3 (default parameters) (Vaser et al. (2017) Genome Res. 27:737-746); this approach has successfully been used to assemble plant, bacterial and fungal genomes (Lee et al. (2019) Plants 8, doi:10.3390/plants8080270; Michael et al. (2018) Nat. Commun. 9, doi:10.1038/s41467-018-03016-2; Giordano et al. (2017) Sci. Rep. 7, doi:10.1038/s41598-017-03996-z; Liao et al. (2019) Front. Microbiol. 10, doi:10.3389/fmicb2019.02068). The improved contigs were finally separated into primary contigs and alternate haplotigs using Purge Haplotigs (Roach et al. (2018) BMB Bioinf 19, doi:10.1186/s12859-018-2485-7).

Reads from the Hi-C assay were aligned to the draft assembly using BWA-MEM v0.7.17 (parameters: −5SP and −t8) (Li (2013) “Aligning sequence reads, clone sequences and assembly contigs with BWA-MEM” arXiv). SAMBLASTER v0.1.224 (default parameters) was used to flag PCR duplicates for later exclusion from the analysis (Faust and Hall (2014) Bioinf 30:2503-2505). Alignments were then filtered with Samtools v1.9 (parameters: -F 2304) to remove non-primary alignments (Li (2009) Bioinf 25:2078-2079). FALCON-Phase v2 (default parameters) was used to correct likely phase switching errors in the primary contigs and alternate haplotigs from Purge Haplotigs and create two complete sets of contigs for each phase (Kronenberg et al. (2018) “FALCON-phase: integrating PacBio and Hi-C data for phased diploid genomes” Biorxiv).

Phase Genomics' Proximo version b4869cc Hi-C genome scaffolding platform was used to create chromosome-scale scaffolds from FALCON-Phase's haplotypes following the single-phase scaffolding procedure as previously described (Ghurye et al. (2017) BMC Genomics 18, doi:10.1186/s12864-017-3879-z). As in the LACHESIS method (Burton et al. (2013) Nat. Biotechnol. 31:1119-1125), this process computes a contact frequency matrix from the aligned Hi-C read pairs, normalized by the number of Sau3AI restriction sites (GATC) on each contig, and constructs scaffolds by optimizing the expected contact frequency and other statistical patterns in Hi-C data. Juicebox v1.9.8 was then used to correct scaffolding errors (Durand et al. (2016) Cell Syst 3:99-101), and FALCON-Phase was run a second time to detect and correct phase switching errors that were not detectable at the contig level. The fully phased, chromosome-scale set of scaffolds were finally polished with Illumina reads (2×150 nt, 177.3 Gb) using Racon v1.4.3. Genome size and the level of heterozygosity were calculated using GenomeScope 2.0 and findGSE v0.1.0 for levels of k ranging from 21 to 81 assuming a diploid model (Ranallo-Benavidez et al. (2019) “GenomeScope 2.0 and Smudgeplots: Reference-free profiling of polyploidy genomes” BioRxiv). The input k-mer abundance histograms were calculated using KmerGenie vi.7051 (FIG. 4D).

### Genome Annotation

Genome annotation was carried out as a service at Keygene. The repeat content in the genome was estimated de novo by parsing through the assembly with RepeatScout v1.0.5 (Price et al. (2005) Bioinf 21:I351-I358). The identified repeats were compared with the non-redundant proteins (NR) and nucleotide (NT) databases from the National Center for Biotechnology Information (NCBI). Repeats were discarded if they had significant matches with sequences in the NR or NT databases unless those sequences contained transposon annotations in their descriptions. The resulting repeat sequences were used to mask the genome assembly with RepeatMasker v4.1.0 prior to the structural gene annotation (Tarailo-Graovac & Chen (2009) Curr. Protoc. Bioinf. 25:4.10.11-14.10.14).

Both haplotypes were annotated individually. Four gene prediction methods were used to identify protein-coding genes including 1) Augustus v3.3.3 software trained using aligned ‘Daphna’ RNA-seq data to generate a species specific model (Rao et al. (2014) BMC Genomics 15, doi:10.1186/1471-2164-15-964; Stanke & Waack (2003) Bioinf 19, doi:10.1093/bioinformatics/btg1080), 2) GeneID v1.4.4 software with the rice.param.Aug_3_2004 gene model (Guigo et al. (1992) J. Mol. Bio. 226:141-157), 3) SNAP software with a rice gene model (Korf (2004) BMC Bioinf. 5, doi:10.1186/1471-2105-5-59), and 4) GlimmerHMM v3.0.4 software also with a rice gene model (Majoros et al. (2004) Bioinf 20:2878-2879).

Expression data was incorporated in the gene prediction pipeline to improve gene prediction and subsequent annotation. RNA-seq data was aligned to the (unmasked) genome and assembled into transcripts using Hisat2 v2.1.0 and Stringtie v2.0.6 (Pertea et al. (2016) Nat. Protoc. 11:1650; Kim et al. (2019) Nat. Biotechnol. 37, doi:10.1038/s41587-019-0201-4). Subsequently, PASA v2.3.3 (Haas et al. (2003) Nucleic Acids Res. 31:5654-5666) alignment assemblies were created based on overlapping transcript alignments and supplied to EvidenceModeler v1.1.1 which is able to provide consensus predictions based on ab initio gene predictions as well as transcriptome alignments (Haas et al. (2008) Genome Biol. 9, doi:10.1186/gb-2008-9-1-r7). Putative functions of the predicted genes were inferred by scanning the TAIR10, Swiss-Prot and TrEMBL databases with the Automated Human Readable Descriptions (AHRD) pipeline (Sato et al. (2012) Nature 485:635-641). Disease resistance gene analogs (RGAs) were identified using Drago2 (Osuna-Cruz et al. (2018) Nucleic Acids Res. 46, doi:10.1093/nar/gkx1119).

### Identification of Vanillin Genes

Candidate genes involved in the vanillin pathway were identified by running BLASTP against each haplotype-specific proteome using previously reported sequences implicated in this pathway (Rao et al. (2014) BMC Genomics 15, doi:10.1186/1471-2164-15-964; Gallage & Møller (2018) in Biotechnology of Natural Products (Springer, Cham)). The set of genes shown with transcript IDs as previously reported (Rao et al. (2014)), or Genbank IDs included phenylalanine ammonia-lyase (PAL, combined.40814), cinnamate 4-hydroxylase (C4H, combined.32468), 4-coumarate CoA ligase (4CL, combined.91179), hydroxycinnamoyl CoA:shikimate hydroxycinnamoyl transferase (HCT, combined.163122), coumaroyl shikimate 3′-hydroxylase (C3′H, combined.55560), caffeoyl-CoA 3-O-methyltransferase (CCoAOMT, combined.78674), cinnamoyl CoA reductase (CCR, combined.5270), caffeoyl shikimate esterase (CSE, combined.4306), and caffeoyl-CoA O-methyltransferase (OMT, AAS64572).

Reads were mapped simultaneously to both haplotypes with Hisat2 v2.1.0 using information on exons and splice sites that was extracted from the corresponding gtf file. The transcripts for each sample were then assembled and merged using the Stringtie v2.0.6 pipeline and the standardized FPKM values for each transcript were calculated using Ballgown (Frazee et al. (2015) Nat. Biotechnol. 33:243-246).

### Identification of RGAs, MADS-Box, and Shattering-Related Genes

Disease resistance gene analogs (RGAs) were identified in the V. planifolia genome including RGA variants in V. pompona and transcript support from a previous study (Rao et al. (2014)). RGAs were identified and classified by running Drago v2 (default parameters) on the proteome derived from both haplotypes (Osuna-Cruz et al. (2018) Nucleic Acids Res. 46, doi:10.1093/nar/gkxl119). The classification was based on the presence of signature domains including Coiled Coil (CC), Kinase (Kin), Leucine rich region (LRR), nucleotide binding site (NBS), Toll-interleukin region (TIR), and transmembrane domain (TM). Multiple RGA proteins that have been associated with Fusarium resistance across multiple species were also scanned for signature domains. These included NP_849908.1 (Arabidopsis thaliana), FOM-2_AAS80152 (Cumulus melo), Bol037156_FOC1 (Brassica oleracea), Bra012688 and Bra012689 (Brassica rapa), AJT39542.1 (Solanum pennellii), AAD47197.1 (Zea mays), AAD27815.1 (Solanum lycopersicum), Q7XBQ9_RGA2 (Solanum bulbocastanum), ABY75802, ACF21694_RGA2, and ACF21695_RGA5 (Musa acuminata malaccensis). Light, dark, mesocarp, placenta, leaf, root, and stem tissues are as previously reported for V. planifolia ‘Daphna’ (Rao et al. (2014)).

To identify MADS-box genes, the Hidden Markov Model (HMM) profile of the MADS-box domain (accession PF00319.18 in the Pfam v2 database) was queried against the genome protein sequences using the hmmsearch tool from HMMER v3.2.1 (default parameters). The phylogenetic trees were generated with MEGA X (Kumar et al. (2018) Mol. Biol. Evol. 35:1547-1549) using the maximum likelihood method and the Whelan and Golden model (Whelan & Goldman (2001) Mol. Biol. Evol. 18:691-699). Initial trees were calculated from genetic distances using the Neighbor-Joining method, and all positions with less than 95% site coverage were eliminated.

The putative shattering-associated proteins were identified through sequence similarity (BLASTP) with known protein sequences from other species, including Arabidopsis thaliana (SHATTERPROOF1, SHATTERPROOF2, INDEHISCENT, ALCATRAZ, FRUITFUL, REPLUMLESS, NST1, SND1, ADPG1, ADPG2, AGAMOUS and SEEDSTICK), Glycine max (NST1A, SHAT1-5 and Dirigent-like protein BAP91522), and Oryza sativa (OsSh1, OsSh2).

### RNAseq Data Analysis

Transcript support was quantified for identified candidates using publicly available RNA-seq data including one study that also used the ‘Daphna’ clone (Rao et al. (2014); Gallage et al. (2014) Nat. Commun. 5, doi:10.1038/ncomms5037). The dataset includes a number of different tissues in the vanilla bean at various developmental stages (8, 10 weeks and 3, 5, 6 months) (Rao et al. (2014)). Prior to mapping the reads, genome-wide exon and splice site information was extracted from the gtf file using the scripts HiSAT2_extract_exons.py and HiSAT2_extract_splice_sites.py from HiSAT2 v2.1.0. This information was then used to construct an index file of the genome assembly with HiSAT2-build v2.1.0 using options-exon and -ss. The reads were mapped simultaneously to both haplotypes with HiSAT2 using the --dta option. To avoid multiple read mapping across haplotypes, non primary alignments were removed from the bam files using samtools vi.9-210-g72d140b (parameters -b -F 256). The transcripts for each sample were then assembled and merged using the Stringtie v2.0.6 pipeline (-e and -B options), and the standardized FPKM values for each transcript were calculated using the R package Ballgown v2.18.0 (Frazee et al. (2015)).

### Identification of Homologous Genes Across Haplotypes

The predicted proteins were clustered into orthogroups with OrthoFinder v2.3.12 (default parameters). Depending on the haplotypic origin of the proteins in the clusters, each orthogroup was classified as containing one to one, many (A) to one (B), one (A) to many (B), many to many, or only haplotype specific proteins. The relative level of transcript abundance for each pair of genes in the one to one orthogroups was evaluated as the log 2 of the ratio FPKM(B)/FPKM(A).

### Quality Assessment and Validation

The completeness of the gene space of the assembly was assessed through the detection of Benchmarking Universal Single-Copy Orthologs (BUSCOs, viridiplantae_odb10, v3.1.0) and by calculating the percentage of mapped reads of the ‘Daphna’ RNA-seq dataset previously published (Simao et al. (2015) Bioinf. 31:3210-3212; Rao et al. (2014)) (Table 1). The genome was aligned with Minimap2 v2.11-r797 to the previously published draft assembly to evaluate the similarities/differences across both assemblies (Hu et al. (2019) Sci. Rep. 9:3416) (FIG. 3). Coverage of short genomic reads along the genome (in 100 Kb non-overlapping windows) was used to identify potentially collapsed regions which can accumulate alignments at much higher depths. Finally, the overall structure of the pseudo molecules was evaluated by visualizing frequency of DNA interaction in a Hi-C contact map using Juicebox v1.9.8 (FIG. 4F).

### Resequencing Analysis

Short read genomic sequences from the seven accessions were processed with fastq-mcf (Aronesty (2011) in Com/p/ea-utils/wiki/fastqmcf) to remove adapters, clip bases with quality lower than 30 (phred-scaled quality score), and eliminate reads that were shorter than 50 nucleotides. The processed reads were then aligned to haplotype A using HiSAT2 v2.1.0 and variants were called with Freebayes v1.3.1-19-g54bf409 (Kim & Salzberg (2015) Nat. Methods 12:357-360; Garrison & Marth (2012) “Haplotype-based variant detection from short-read sequencing” arXiv). All 18,028,080 unfiltered variants were annotated with SNPeff (default parameters) to predict their impact on specific genes across the different accessions (FIG. 4E) (Cingolani et al. (2012) Fly 6:80-92).

### GBS Analysis

The raw GBS fastq files from a previous study (Hu et al. (2019) Sci. Rep. 9:3416) were processed and aligned against haplotype A following the same procedure as for the resequencing analysis. Called variants were then filtered to keep only bi-allelic SNPs (BCFtools v1.9-271-gbc0909e with parameters -m2-M 2-types snps) with quality of 30 or higher (phred-scaled quality score for the assertion made on the alternative allele) and depths between 5 and 1,000 (VcfFilter v1.0.0 with parameters “QUAL >29”-g “DP >4”-g “DP<1000”) (Garrison & Marth (2012)). SNPs were further filtered to retain those with minor allele frequencies equal or greater than 10% that were covered by at least 70% of the individuals (VCFtools v0.1.17 with parameters --maf 0.1--max-missing 0.7) (Danecek et al. (2011) Bioinformatics 27:2156-2158). Finally, only SNPs that were at least 1 kb apart from each other were retained to avoid oversampling sites in linkage disequilibrium (VCFtools with parameter --thin 1000). Genetic distances on the filtered set of SNPs were estimated using TASSEL v5.2.59 (Bradbury et al. (2007) Bioinf 23:2633-2635) (-DistanceMatrixPlugin) and Principal Component Analysis (PCA) was conducted with the function prcomp from the stats v3.6.2 R package. The UPGMA tree was estimated with the aboot function from the poppr v.2.6.1 R package using 500 bootstrap replicates. The tree was rooted on the V. mexicana accessions AC191 and AC192 using the root function from the ape v5.4 R package.

### Comparative Genomics

The V. planifolia ‘Daphna’ genome was compared against related comparator genomes that included gymnosperm outgroup Ginko biloba (gingko), basal angiosperm Amborella trichopoda, nine selected non-orchid monocots including Asparagus officinalis (asparagus), Musa acuminata (banana), Phoenix dactylifera (date palm), Spirodela polyrhiza (duckweed), Elaeis guineensis (oil palm), Ananas comosus (pineapple), Oryza sativa (rice), Sorghum bicolor (sorghum), and Zostera marina (seagrass), four selected eudicots including Arabidopsis thaliana, Vitis vinifera (grape), Populus trichocarpa (poplar) and Nelumbo nucifera (sacred lotus), and three selected orchid genomes Apostasia shenzhenica (Zhang et al. (2017) Nature 549:379-383), Phalaenopsis equestris (Cai et al. (2015) Nat. Genet. 47:65-72), and Dendrobium catenum (Zhang et al. (2016) Sci. Rep. 6, doi:10.1038/srep19029), along with the seven vanilla species (V. planifolia ‘Daphna’, V. planifolia ‘Guy 1’, V. planifolia ‘Hawaii’, V. planifolia ‘Painter’, V. x tahitensis ‘Haapape’, V. pompona ‘King’, and V. mexicana ‘Sheila’) for a total of 25 representative plant taxa with publicly available genomes. All vanilla genome datasets were generated as part of this study, while most non-vanilla genome datasets were downloaded from Phytozome v12.1.5 release when available (Goodstein et al. (2012) Nucleic Acids Res. 40, doi:10.1093/nar/gkr944), with the remaining datasets downloaded from Genbank.

### Construction of Orthologous Groups and Inference of Species Tree

OrthoFinder (version 2.3.8) was used with a set of input protein sequences derived from primary transcripts from each of the 25 selected comparator genomes to analyze orthologous groups (Emms & Kelly (2015) Genome Biol. 16, doi:10.1186/s13059-015-0721-2). A total of 26 single-copy orthologous groups that were strictly single copy in each of the taxa were selected. The protein sequences for these orthologous gene families were aligned using MAFFT accurate option (L-INS-i) and concatenated into a single supermatrix with 25 taxa and 17,506 sites using FASconCAT-G (Katoh et al. (2005) Nucleic Acids Res. 33:511-518; Kueck & Longo (2014) Front. Zool. 11, doi:10.1186/s12983-014-0081-x). The maximum likelihood (ML) tree was inferred using RAxML-NG with the JTT model and bootstrap analysis were performed with 200 replicates (Stamatakis (2014) Bioinf 30:1312-1313). Bayesian estimation of species divergence times were performed using MCMCTree in the PAML package, using fossil constraints under molecular clock models (Yang (2007) Mol. Biol. Evol. 24:1586-1591). The Hessian matrix was empirically estimated using WAG+Gamma model to improve the accuracy of MCMCTree. For the fossil calibrations, the flowering plant fossil records compiled previously (Zhang et al. (2020) Nature 577:79-84) were followed. Specifically, the estimated age for the various fossils corresponding to the crown group of Eudicots, Proteales, Zingiberales, Poales and Mesangiosperms were used as constraints to calibrate the species divergence.

### Inference of Synteny Blocks

Syntenic blocks within and among genomes were inferred based on the QUOTA-ALIGN pipeline implemented in the JCVI package (on the world wide web at github.com/tanghaibao/jcvi) (Tang et al. (2011) BMC Bioinf 12, doi:10.1186/1471-2105-12-102). Briefly, the coding sequences (CDS) of protein-coding genes from each comparator genome were extracted and compared in an all-against-all fashion using LAST (Kielbasa et al. (2011) Genome Res. 21:487-493), with weak scoring pairs and pairs due to tandem gene duplications removed prior to clustering. Weak scoring pairs are defined as gene pairs with a C-score of less than 0.7, and tandem duplicate pairs are defined as pairs due to proximal gene duplications within 10 gene distance from one another. C-score generalizes the concept of mutual best hit, where the mutual best hit would have a C-score value of 1, and a cutoff of 0.7 implies that matches were excluded that were <70% similar to the best match in either genome. An additional filtering step was used to remove LAST matches over 98% sequence similarity prior to the C-score filtering when comparing a genome against itself, in order to preclude inference of genomic events that would otherwise appear too recent.

Clustering of syntenic anchors were then identified through a single linkage algorithm that would place the gene pairs in the same block if they were within a preset distance cutoff of 30 genes apart from another gene pair. These clustered gene pairs were then referred to as “anchor pairs”. All blocks that contain more than four syntenic anchor pairs are retained for further analysis. Only V. planifolia haplotype A was used for the syntenic comparisons, since haplotypes A and B are largely collinear and show almost identical patterns when comparing across species.

### Dating of Genome Duplication Events

The Ks distance was calculated between the gene pairs based on a pipeline implemented in the JCVI package (on the world wide web at github.com/tanghaibao/jcvi). Briefly, the coding sequences from the gene pairs were aligned codon-by-codon, using PAL2NAL (Suyama et al. (2006) Nucleic Acids Res. 34, doi:10.1093/nar/gkl315). To calculate Ks, the Nei-Gojobori method was used implemented in yn00 program as part of the PAML package (Yang et al. (2007) Mol. Biol. Evol. 24:1586-1591).

### Data Availability

Publicly available data sets were re-analyzed using the complete ‘Daphna’ genome and included SRX286672 (unspecified V. planifolia accession) (Gallage et al. (2014) Nat. Commun. 5, doi:10.1038/ncomms5037), data from V. planifolia ‘Daphna’ PRJNA253813 (Rao et al. (2014) BMC Genomics 15, doi:10.1186/1471-2164-15-964), and GBS data from RJNA507246 (Hu et al. (2019) Sci. Rep. 9:3416).

### 1.2 the V. planifolia Genome Reveals Haplotype Differences

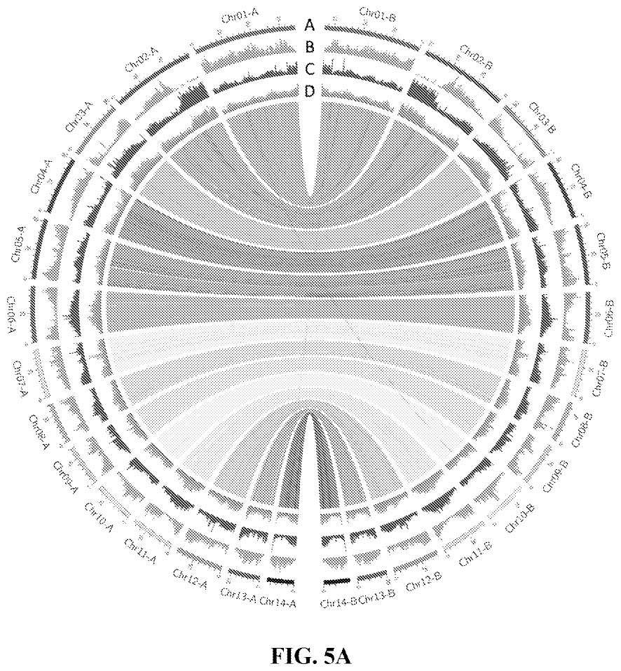

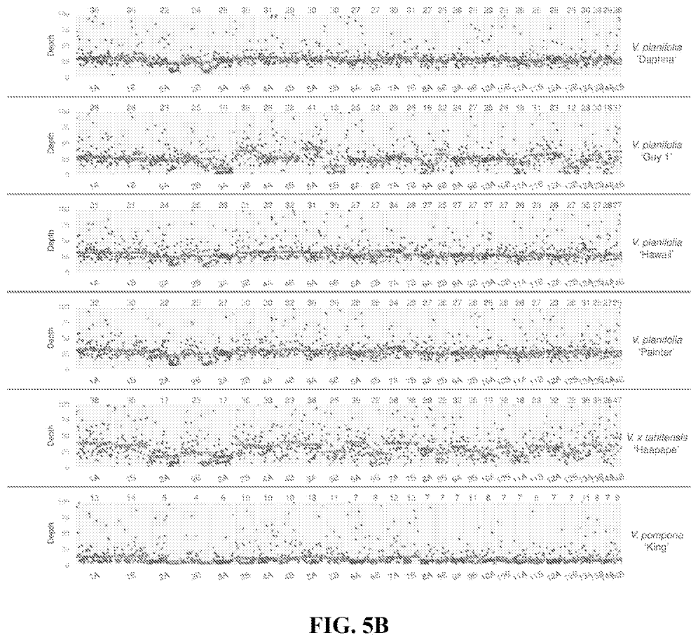

A phased, diploid reference genome for V. planifolia ‘Daphna’ (‘Daphna’) was assembled de novo from a combination of Oxford Nanopore Technologies (ONT) long reads and Illumina short reads to produce highly contiguous pseudochromosomes (FIG. 5A). The ‘Daphna’ clone was selected for sequencing because it represents the most common commercial type of vanilla and there is existing genomics information available for this clone (Hu et al. (2019)). Using Hi-C chromatin contact data, 86% of contig sequences were organized on the 14 chromosomes for each of the two haplotypes and the chromosomes were numbered sequentially by length (FIG. 5B). Genic completeness was estimated at 93.9% for both haplotypes using the conserved BUSCO gene set which was supported by a 95.2% mapping rate of RNA-seq reads from a separate ‘Daphna’ RNA-seq dataset (Simao et al. (20150) Bioinf 31:3210-3212) (Table 1).

TABLE 1
Summary statistics of the V. planifolia ‘Daphna’ assembly compared to the
previously published Vapla0.1.4 draft assembly (Hu et al. (2019) Sci. Rep. 9: 3416).

| | Haplotypes | | | |
|---|---|---|---|---|
| Assembly | A | B | A + B | Vapla0.1.4 |

General stats

| | | | | |
|---|---|---|---|---|
| Total length (Mb) | 736.8 | 744.2 | 1,480.9 | 2,203.6 |
| Contigs/scaffolds (#) | 668 | 668 | 1,336 | 794,534 |
| Largest contig (Mb) | 88.30 | 83.42 | 88.30 | 0.63 |
| N50 (Mb) | 42.01 | 40.79 | 42.00 | 0.05 |
| N90 (Mb) | 0.5 | 0.5 | 0.5 | 0.000073 |
| L50 (# Contigs/scaffolds) | 7 | 7 | 13 | 9,595 |
| L90 (# Contigs/scaffolds) | 47 | 56 | 102 | 169,623 |
| % N's | 0.0183 | 0.0181 | 0.0182 | 19.947 |

Genomic short reads alignment

| | | | | |
|---|---|---|---|---|
| Overall alignment rate | 72.9 | 73.5 | 78.6 | 83.24 |
| Concordantly 1 time | 51.5 | 52.1 | 39.5 | 41.17 |
| Concordantly >1 times | 12.6 | 13.3 | 31.1 | 30.48 |

RNA-seq read alignment

| | | | | |
|---|---|---|---|---|
| Overall alignment rate | 91.8 | 91.6 | 95.2 | 96.7 |
| Concordantly 1 time | 76.2 | 76.3 | 44.8 | 38.1 |
| Concordantly >1 times | 10.4 | 10.2 | 46.0 | 52.9 |

BUSCOs in genome

| | | | | |
|---|---|---|---|---|
| Complete BUSCOs | 91.0 | 89.3 | 93.9 | 84.9 |
| Single-copy | 86.8 | 84.7 | 25.2 | 52.7 |
| Duplicated | 4.2 | 4.9 | 68.7 | 32.2 |
| Fragmented BUSCOs | 3.3 | 5.2 | 1.4 | 6.6 |
| Missing BUSCOs | 5.7 | 5.2 | 4.7 | 8.5 |

BUSCOs in gene models

| | | | | |
|---|---|---|---|---|
| Complete BUSCOs | 81.2 | 80.2 | 90.6 | — |
| Single-copy | 70.4 | 70.1 | 27.5 | — |
| Duplicated | 10.8 | 10.1 | 63.1 | — |
| Fragmented BUSCOs | 12.0 | 13.2 | 6.1 | — |
| Missing BUSCOs | 6.8 | 6.6 | 3.3 | — |

A total of 29,167 and 29,180 genes were identified in haplotypes A and B (randomly assigned), respectively (Table 2).

TABLE 2
Summary statistics of gene models from the complete
V. planifolia ‘Daphna’ genome.

| | Haplotype | Haplotype |
|---|---|---|
| Statistic | A | B |
| Number of genes | 29,167 | 29,180 |
| Number of multiexon genes | 22,052 | 22,117 |
| Number of genes with functional annotation | 22,026 | 22,086 |
| Number of genes without functional annotation | 7,141 | 7,094 |
| Number of exons | 140,562 | 140,791 |
| Mean exon length (bp) | 203 | 203 |
| Mean number of exons per gene | 4.8 | 4.8 |
| Mean intron length (bp) | 2,293 | 2,255 |
| Mean coding sequence length (bp) | 983 | 985 |
| Shortest protein size (AA) | 49 | 49 |
| Mean protein size (AA) | 327 | 327 |
| Longest protein size (AA) | 5,343 | 4,253 |
| Orthogroups 1 to 1 | 11,208 | 11,208 |
| Orthogroups 1 (A) to many (B) | 2,628 | 5,920 |
| Orthogroups many (A) to 1 (B) | 5,652 | 2,460 |
| Orthogroups many to many | 5,690 | 5,621 |
| Orthogroups haplotype A specific | 714 | — |
| Orthogroups haplotype B specific | — | 576 |
| Orthogroups unassigned genes | 3,275 | 3,395 |

Summary statistics of the long-read, haplotype assembly compared to intermediate assemblies and the previous ‘Daphna’ draft genome showed substantial improvements in contiguous sequence length (Hu et al. (2019)) (Tables 1 and 3).

TABLE 3
Assembly statistics for intermediate assemblies.

| | Raw | ONT Polished | Phased Assembly | | | Illumina Polished Assembly | | |
|---|---|---|---|---|---|---|---|---|
| | Contigs | | Scaffolds | | | | | |
| Assembly | A + B | A + B | A | B | A + B | A | B | A + B |

General stats

| | | | | | | | | |
|---|---|---|---|---|---|---|---|---|
| Total length (Mb) | 1,301.7 | 1,352.9 | 736.9 | 744.3 | 1,481.2 | 736.8 | 744.2 | 1,480.9 |
| Contigs/scaffolds (#) | 4,496 | 4,496 | 668 | 668 | 1,336 | 668 | 668 | 1,336 |
| Largest contig (Mb) | 2.1 | 2.2 | 88.3 | 83.4 | 88.3 | 88.3 | 83.4 | 88.3 |
| N50 (Mb) | 0.4 | 0.4 | 42.0 | 40.8 | 42.0 | 42.0 | 40.8 | 42.0 |
| N75 (Mb) | 0.2 | 0.2 | 35.3 | 37.4 | 35.3 | 35.3 | 37.5 | 35.3 |
| L50 (# Contigs/scaffolds) | 1,091 | 1,086 | 7 | 7 | 13 | 7 | 7 | 13 |
| L75 (# Contigs/scaffolds) | 2,226 | 2,217 | 11 | 11 | 22 | 11 | 11 | 22 |
| GC (%) | 31.4 | 30.6 | 30.6 | 30.6 | 30.6 | 30.8 | 30.8 | 30.8 |
| # N's per 100 kbp | 0.0 | 0.0 | 18.2 | 18.0 | 18.1 | 18.3 | 18.1 | 18.2 |

BUSCOs percentages

| | | | | | | | | |
|---|---|---|---|---|---|---|---|---|
| Complete BUSCOs | 0.0 | 84.7 | 70.1 | 73.4 | 79.1 | 91.0 | 89.3 | 93.9 |
| Single-copy | 0.0 | 55.1 | 68.2 | 70.8 | 32.7 | 86.8 | 84.7 | 25.2 |
| Duplicated | 0.0 | 29.6 | 1.9 | 2.6 | 46.4 | 4.2 | 4.9 | 68.7 |
| Fragmented BUSCOs | 3.5 | 9.4 | 16.7 | 14.4 | 11.3 | 3.3 | 5.2 | 1.4 |
| Missing BUSCOs | 96.5 | 5.9 | 13.2 | 12.2 | 9.6 | 5.7 | 5.2 | 4.7 |

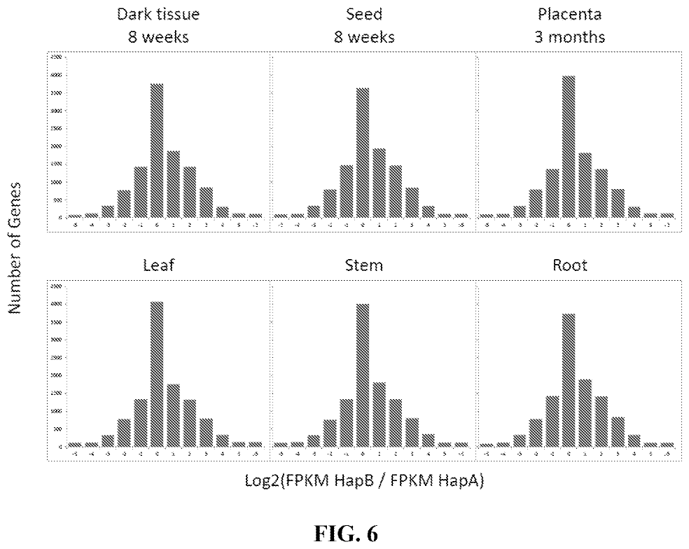

Introns in ‘Daphna’ were longer than in most other plant species with 13% exceeding 5 kb (Table 2, FIG. 4A) similar to intron length patterns reported in all other orchids with sequenced genomes (Cai et la. (2015) Nat. Genet. 47:65-72; Zhang et al. (2016) Sci. Rep. 6, doi:10.1038/srep19029; Zhang et al. (2017) Nature 549:379-383; Chao et al. (2018) Plant Biotechnol. J. 16:2027-2041). OrthoFinder analysis indicated that 11,208 orthogroups out of 18,948 had a 1:1 relationship between haplotypes A and B (Table 2). Approximately 50% of the 11,208 orthogroups showed greater than 2-fold differences in transcript abundance (FIG. 6).

The final assembled genome length was 1,480.9 Mb (736.8 Mb and 744.2 Mb for haplotypes A and B, respectively) with haplotypes A and B (randomly assigned) structurally similar and largely collinear to each another (FIG. 5A, FIG. 3). Chromosome staining confirmed 28 chromosomes for ‘Daphna’, and flow cytometry of meristem nuclei indicated 2C=4.87 pg (4.76 Gb) and 4.30 pg (4.21 Gb) using pea and wheat references, respectively, similar to previous reports (Brown et al. (2017) Genome Biol. Evol. 9:1051-1071; Bory et al. (2008) Genome 51:816-826; Lepers-Andrzejewski et al. (2011) Am. J. Bot. 98:986-997) (FIG. 2). The duplicated portion of the 2E nuclei was 25.9% similar to the previously reported replicate fraction P=28.4% for V. planifolia (Brown et al. (2017)). Haploid genome size estimation by kmer analysis ranged from 637.3 to 733.1 Mb in close agreement to the final assembled genome length (Table 4).

TABLE 4
Genome size estimation by kmer analysis using Jellyfish with GenoScope (G_Scope) and
Jellyfish with findGSE (FindGSE). Kmer sizes from 21 to 81 bp are shown. Repeat %
and heterozygosity are shown for each kmer size and genome size estimation tool.

| | Haploid length (Mb) | | Repeats (%) | | Heterozygosity (%) | |
|---|---|---|---|---|---|---|
| Kmer size (bp) | G Scope | FindGSE | G_Scope | FindGSE | G_Scope | FindGSE |
| 21 | 708.1 | 733.1 | 40.4 | 35.7 | 2.47 | 1.97 |
| 31 | 683.4 | 706.7 | 33.4 | 30.0 | 2.37 | 1.72 |
| 41 | 662.0 | 686.7 | 31.2 | 27.8 | 2.24 | 1.51 |
| 51 | 648.7 | 695.3 | 30.3 | 26.9 | 2.14 | 1.25 |
| 61 | 642.3 | 691.3 | 30.0 | 26.5 | 2.06 | 1.09 |
| 71 | 639.7 | 699.4 | 30.0 | 26.1 | 1.99 | 0.93 |
| 81 | 637.3 | 677.6 | 30.1 | 25.9 | 1.95 | 0.96 |

The disparity between genome size as estimated by flow cytometry and the resulting assembly could be the result of a high abundance of repetitive elements, though long read sequencing technology should reduce this impact. Approximately 46.2% of the assembly consisted of repetitive sequences, which included 20.8% retrotransposons and 17.0% DNA transposons (Table 5).

TABLE 5
Repeat content of V. planifolia ‘Daphna’.

| | Number | | Total length (Mb) | | % of Assembly | |
|---|---|---|---|---|---|---|
| | Haplotype | Haplotype | Haplotype | Haplotype | Haplotype | Haplotype |
| Type | A | B | A | B | A | B |
| Total interspersed | 895,856 | 906,704 | 326.2 | 329.9 | 44.3 | 44.3 |
| repeats | | | | | | |
| Retroelements | 412,249 | 417,677 | 153.0 | 155.5 | 20.8 | 20.9 |
| SINEs | 2,017 | 2,034 | 0.2 | 0.2 | 0.0 | 0.0 |
| LINEs | 242,301 | 245,464 | 79.1 | 80.0 | 10.7 | 10.8 |
| LTR | 167,931 | 170,179 | 73.8 | 75.3 | 10.0 | 10.1 |
| DNA transposons | 325,175 | 329,001 | 125.3 | 126.0 | 17.0 | 16.9 |
| Unclassified | 158,432 | 160,026 | 48.0 | 48.5 | 6.5 | 6.5 |
| Simple repeats | 157,101 | 158,963 | 12.0 | 12.2 | 1.6 | 1.6 |

An alternative hypothesis is that partial endoreduplication could impact species-specific chromatin rearrangement increasing flow cytometry fluorophore intercalation resulting in an inflated genome size estimation (Brown et al. (2017) Genome Biol. Evol. 9:1051-1071). Overall, the goal of capturing V. planifolia genic space was realized and the discrepancy between flow cytometry and genome sequence length remains an open area for future inquiry.

### 1.3 Evolution of the Vanilla Genome

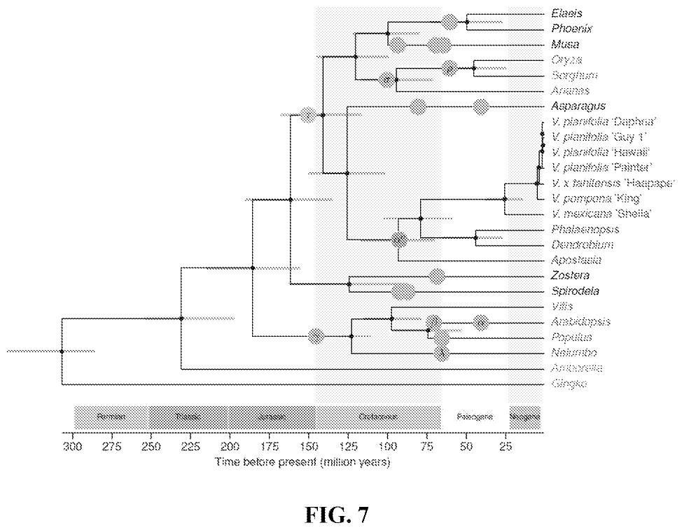

Comparative genomics provides a valuable framework to utilize findings from model organisms to study less-well-understood systems such as vanilla. A total of 25 representative plant taxa which included seven Vanilla genomes, three orchid genomes including Apostasia (Zhang et al. (2017) Nature 549:379-383), Phalaenopsis (Cai et al. (2015) Nat. Genet. 47:65-72), and Dendrobium (Zhang et al. (2016) Sci. Rep. 6, doi:10.1038/srep19029), the gymnosperm outgroup gingko, basal angiosperm Amborella, nine selected non-orchid monocots, and four selected eudicots were compared (FIG. 7). Analysis showed that the phylogenetic placement of Vanilla fell within the orchid family as expected, which included Apostasia as the earliest diverging orchid lineage among the selected taxa while Vanilla was closer to the clade consisting of Phalaenopsis and Dendrobium than Apostasia (Zhang et al. (2017) Nature 549:379-383). Bayesian estimation of species divergence time suggested that Apostasia diverged from the other orchids ˜90 million years ago (Mya), while Vanilla diverged from the lineage of Phalaenopsis and Dendrobium ˜77 Mya. Among the Vanilla taxa sampled, V. mexicana represented the earliest diverging branch ˜25 Mya, while V. pompona diverged from V. planifolia and V. x tahitensis ˜4 Mya, which is consistent with the overall trend from the vanilla resequencing data.

Whole genome duplication (WGD) plays a significant role during orchid evolution and was therefore investigated using the V. planifolia genome. It was discovered that WGD was a major force in shaping the gene repertoire of the vanilla genome.

Absolute dating of WGD events using inferred orthologs as well as the anchor pairs further supported the WGD events. Synonymous substitutions per synonymous site (Ks) between the syntenic orthologs between Vanilla and other orchids, monocots, eudicot and basal angiosperm Amborella closely followed their increasing phylogenetic distance, as expected (FIG. 7). Specifically, the divergence between Vanilla and related orchids (Apostasia and Phalaenopsis) were between 0.75 to 1.0, with the Apostasia-Vanilla Ks peak greater than Phalaenopsis-Vanilla Ks due to their close phylogenetic relationship. The pan-orchid WGD was identified to be close to the divergence of major orchid clades based on the Ks divergence between the gene pairs. This suggested that the WGD could date back ˜90 Mya, similar to the divergence time of the earliest branching orchid lineage, Apostasia. It is possible that the pan-orchid WGD and orchid diversification could have occurred in close succession, and might have been a precondition for the radiation of the orchids.

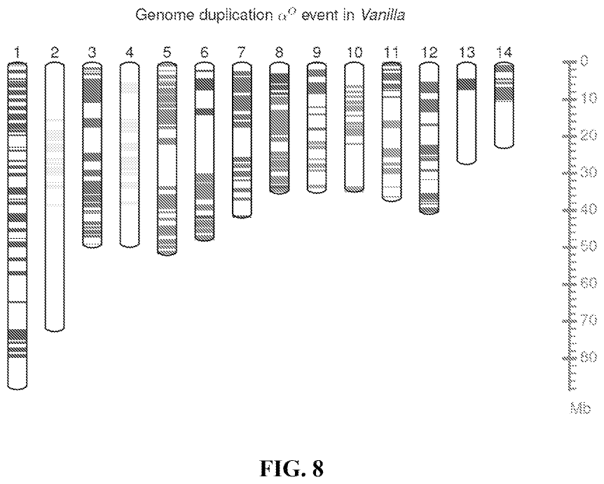

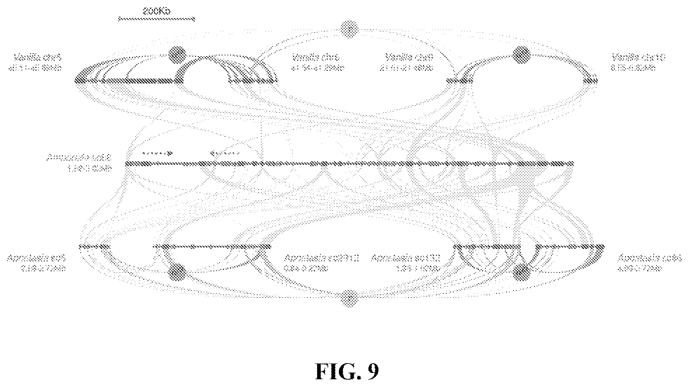

In addition to the temporal evidence, intra-genomic syntenic analyses of Vanilla shows clear structural evidence of at least one major WGD event that covers all chromosomes (FIG. 8). Structural comparison of V. planifolia haplotype A versus itself revealed a total of 1,458 gene pairs derived from whole genome duplication events, previously inferred as an orchid-wide WGD event (Cai et al. (2015) Nat. Genet. 47:65-72; Zhang et al. (2017) Nature 549:379-383). Following previous nomenclature, the pan orchid WGD is herein referred to as α^{O}. Collectively, these colinear blocks span 44% of the annotated gene space and involve each of the 14 Vanilla chromosomes. Notably, Vanilla chromosomes two and four appear to be colinear across their entire length. Additionally, syntenic depth analyses indicated that 8% of the Vanilla genome has more than one duplicated segment within the same genome, which would be expected if more than one WGD occurred in the Vanilla lineage (Paterson et al. (2012) Nature 492:423-427; Tang et al. (2010) P. Natl. Acad. Sci. USA 107:472-477). Based on the synonymous substitutions per synonymous site (Ks, FIG. 4B) between the syntenic gene pairs, the pan-orchid ao WGD could date to ˜90 Mya, similar to the divergence time of the earliest branching orchid lineage, Apostasia (Cai et al. (2015); Zhang et al. (2016); Zhang et al. (2017) Nature 549:379-383) (FIG. 8). The orchid-wide ao WGD and orchid diversification could have occurred in close succession, and might have been a precondition for the radiation of the orchids (Cai et al. (2015); Zhang et al. (2017)). Although the exact nature of additional WGD before the orchid α^{O} event is less clear, past evidence suggested that it might be the T-WGD event shared by all monocots except Alismatales (Wang et al. (2014) Nat. Commun. 5, doi:10.1038/ncomms4311; Ming et al. (2015) Nat. Genet. 47:1435-1442). This is supported via the comparison against the Amborella genome, the most basal angiosperm taxon that lacks recent WGDs, where either Vanilla or Apostasia showed a pattern up to four regions as syntenic to a single region in Amborella (FIG. 9). The syntenic patterns between Vanilla and other orchids like Apostasia remain ‘multiple-to-multiple’ (in contrast to the ‘one-to-one’ pattern as would be expected from the shared WGDs), suggesting that the loss of duplicate genes was extensive and occurred largely independently in different orchid lineages. In particular, there might be a limited level of gene loss, or ‘fractionations’, in the shared lineage following the WGD prior to the diversification of orchids. If gene fractionation after the α^{O}-WGD event had occurred in the common lineage that predated the divergence of Vanilla and other orchids, then strong 1-to-1 syntenic regions with a few weak matches would have been observed. The fractionation rate of duplicate genes is also unusually high for the α^{O}-WGD event with retained pairs ranging from 1,458 in Vanilla, 1,044 in Apostasia, and 318 in Phalaenopsis. The fractionation rate difference between different orchids following the ancient polyploidy event may be explained by the different levels of sequence continuity. Overall, the number of retained WGD gene pairs in all three orchids are much lower than the number of gene pairs in other monocot lineages experiencing WGDs of similar age, including a-WGD event in Poales (Ming et al. (2015) Nat. Genet. 47:1435-42) and the α^{SP}/β^{SP} WGDs in Alismatales (Wang et al. (2014) Nat. Commun. 5, doi:10.1038/ncomms4311). Additionally, the orchid-wide WGD gene pairs that were identified between haplotypes A and B are ˜20% more than using either A or B self-comparison alone (3390 A-B′ and A′-B pairs, compared with 1458 A-A pairs'+1344 B-B′ pairs). This illustrates that the haplotype-resolved assembly has a higher sensitivity to resolve WGD events since genes lost in one haplotype could be retained in the other haplotype.

Overall, the ancestral ploidy level for V. planifolia is the same as the other orchid genomes sequenced thus far including Apostasia, Phalaenopsis, and Dendrobium (Cai et al. (2015); Zhang et al. (2016); Zhang et al. (2017)). The presently disclosed high quality V. planifolia genome better clarifies the key events in early orchid evolution that set the context for understanding the gene family dynamics of many functional genes in vanilla.

### 1.4 Resequencing Uncovers Diversity

Six vanilla accessions from four species (V. planifolia, V. pompona, V. x tahitensis, and V. mexicana) were resequenced to investigate genetic diversity and identify genes associated with specific traits (Table 6).

TABLE 6
Accession-specific variants identified from resequencing V. planifolia, V. x tahitensis, and V. pompona. Variants shown are
exclusive and not shared with any other accession.

| | V. planifolia | | | | | | | | V. x tahitensis | | V. pompona | |
|---|---|---|---|---|---|---|---|---|---|---|---|---|
| | Daphna | | Guy1 | | Hawaii | | Painter | | Haapape | | King | |
| Type | Number | % | Number | % | Number | % | Number | % | Number | % | Number | % |
| Unique | 170,985 | 100.0 | 192,281 | 100.0 | 237,256 | 100.0 | 263,839 | 100.0 | 3,387,523 | 100.0 | 3,325,165 | 100.0 |
| variants | | | | | | | | | | | | |
| Heterozygous | 157,440 | 92.1 | 157,280 | 81.8 | 229,788 | 96.9 | 257,026 | 97.4 | 2,511,648 | 74.1 | 644,538 | 19.4 |
| Homozygous | 13,545 | 7.9 | 35,001 | 18.2 | 7,468 | 3.1 | 6,813 | 2.6 | 875,875 | 25.9 | 2,680,627 | 80.6 |
| Ref. allele | 0 | 0.0 | 0 | 0.0 | 0 | 0.0 | 0 | 0.0 | 0 | 0.0 | 0 | 0.0 |
| Alt. allele 1 | 13,541 | 7.9 | 34,962 | 18.2 | 7,462 | 3.1 | 6,808 | 2.6 | 875,622 | 25.8 | 2,678,748 | 80.6 |
| Alt. allele 2 | 4 | 0.0 | 37 | 0.0 | 6 | 0.0 | 5 | 0.0 | 249 | 0.0 | 1,841 | 0.1 |
| Alt. allele 3 | 0 | 0.0 | 2 | 0.0 | 0 | 0.0 | 0 | 0.0 | 4 | 0.0 | 38 | 0.0 |

The genomes were sequenced at an average depth of 117X (based on the size of the assembly of haplotype A) using Illumina short reads. V. planifolia had fewer unique homozygous variants among accessions (6,813-35,001) compared to V. x tahitensis (>875,000), and V. pompona (>2.6M) as expected (Table 4). The SNP density between ‘Daphna’ haplotypes A and B was estimated to be 0.67 SNPs per 100 bp, with 0.53 SNPs per 100 bp in the coding regions.

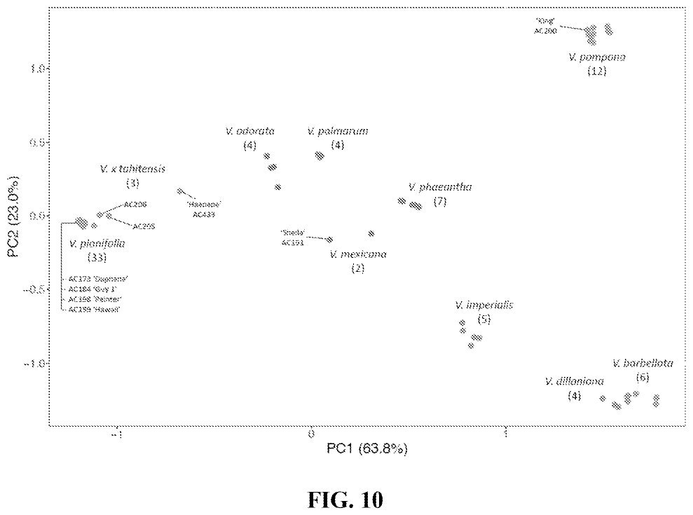

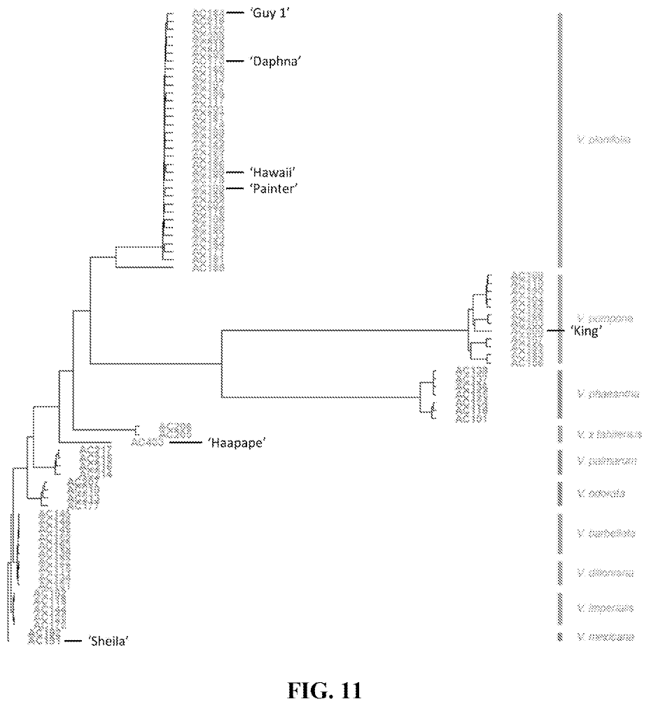

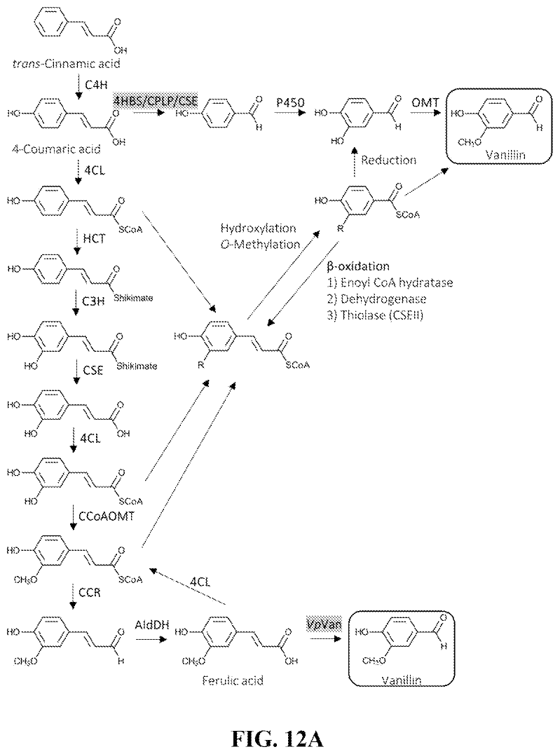

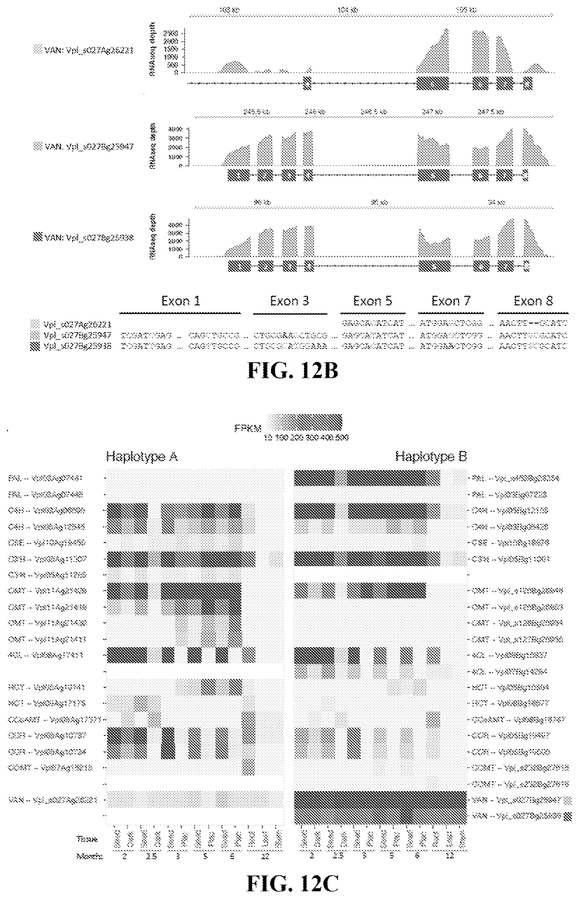

Approximately 50% of the variants identified from resequencing were located in intergenic regions with ˜21% in introns, while ˜1.7% were located in exons. Phylogenetic analyses based on single-copy genes and re-analysis of genotyping-by-sequencing data from a vanilla diversity collection (Hu et al. (2019) Sci. Rep. 9:3416) provided additional insights when mapping against the phased genome. V. x tahitensis ‘Haapape’ is a clone in commercial production in islands of the south Pacific, and is reported to be a hybrid between V. planifolia and V. odorata (Lubinsky et al. (2008) Am. J. Bot. 95:1040-1047). The genotype of ‘Haapape’ is split more evenly between V. planifolia and V. odorata (FIG. 10, FIG. 11) than previously identified hybrids (AC205 and AC206) between V. planifolia and V. odorata. Resequencing of V. planifolia ‘Guy 1’ showed preferential read mapping to chromosomes 3B, 5A, and 12B compared to 3A, 5B, and 12A, respectively, but this was not the case with V. planifolia ‘Hawaii’ as a comparator (FIG. 5B). A similar observation was made for V. x tahitensis ‘Haapape’ for chromosome 3. This may be the result of ‘Guy 1’ and ‘Daphna’ sharing parentage or introgression between the two accessions in recent generations leading to significant haplotype sharing that are consistent with the preferential read mapping seen in those shared chromosomes.

### 1.5 Haplotype-Specific Gene Contributions to Vanillin Biosynthesis

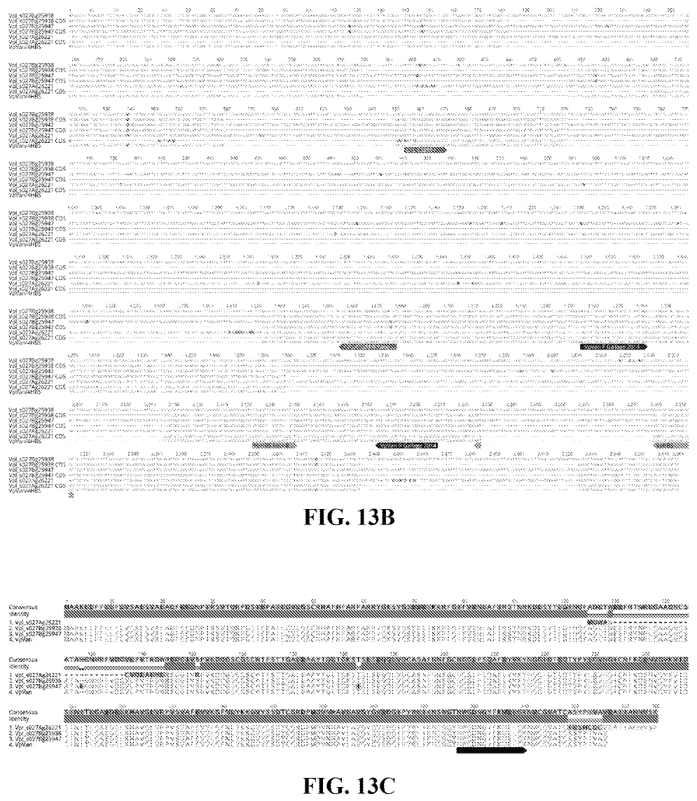

The predicted vanillin pathway (FIG. 12A) was used to investigate the haplotypes in the assembled genome by comparing allelic transcript abundances across different developmental stages and tissue types (FIG. 12B) (Yang et al. (2017) Phytochem. 139:33-46; Rao et al. (2014)). The list of genes for each enzyme in the vanillin pathway, as previously proposed (Rao et al. (2014); Gallage & Møller (2018) in Biotechnology of Natural Products (Springer, Cham)), was narrowed down to their best matches in the genome based on alignment quality. In some cases, there were multiple putative paralogs across haplotypes. In general, most gene transcripts were highly abundant in different tissues of the vanilla beans. As an example, O-methyl transferases (OMTs) are important in the biosynthesis of secondary metabolites including vanillin and lignin (Yang et al. (2017); Gallage & Møller (2018)). Most of the selected OMTs increased in abundance as the beans approached maturity including Vpl_s126Bg26946 designated as OMT4 in a previous study that showed tissue specificity correlated with vanillin biosynthesis (Widiez et al. (2011) Plant Mol. Biol. 76:475-488). Some genes exhibited preferential allele expression. For example, phenylalanine ammonia-lyase (PAL) is the first enzyme in the phenylpropanoid pathway in the proposed route for vanillin biosynthesis. PAL transcript abundance was previously correlated with vanillin abundance (Fock-Bastide et al. (2014) Plant Physiol. Biochem. 74:304-314). PAL transcripts were highly abundant for a single homolog of the four annotated PAL genes, and several OMT homologs also showed differences when comparing transcript abundances among the alleles. Resequencing of the V. planifolia, V. x tahitensis, and V. pompona accessions showed that only a single PAL allele (gene Vpl_s453Bg28354) was present in each accession suggesting that this may be the functional allele in the upstream ferulic acid/vanillin pathway (FIG. 13A). Breeding or genome modifications to obtain the haplotype B PAL allele in the homozygous state could be one route to increasing vanillin content in beans.

### 1.6 Improved Resolution of the Vanillin Biosynthetic Pathway

One enzyme, a cysteine protease-like protein (CPLP, also called vanillin synthase, VpVan, or 4HBS) has been implicated in vanillin biosynthesis, but its potential role in the vanilla plant has not been resolved. The two models of vanillin biosynthesis suggest that CPLP either directly converts ferulic acid to vanillin or converts 4-coumaric acid to 4-hydroxybenzaldehyde leading to vanillin biosynthesis (Yang et al. (2017); Gallage et al. (2014) Nat. Commun. 5, doi:10.1038/ncomms5037) (FIG. 12A). In the ‘Daphna’ genome assembly, Vpl_s027Bg25938 on haplotype B was identified as CPLP as well as a putative paralog on haplotype B (Vpl_s027Bg25947) and the alternative allele on haplotype A (Vpl_s027Ag26221) (FIG. 12B). The ‘Daphna’ genome permitted improvement upon limitations in previous CPLP studies which relied on sequence specificity such as RT-PCR or CPLP antibodies. One study utilized RNA-seq data from an alternative, unnamed 6-month old V. planifolia bean tissue that did not identify all CPLP alleles and paralogs as it did not contain reads that mapped to Vpl_s027Ag26221. Specifically, the in situ PCR primers are predicted to amplify all three CPLP genes mentioned above (Gallage et al. (2014)) (Table 7, FIG. 13B).

TABLE 7
RNA-seq support of three CPLP genes identified in the complete V. planifolia
‘Daphna’ genome. RNA-seq data are based on previous studies that included pod tissues at 6
months (6 m) or 8 weeks (8 w) and 5 months (5 m) (Gallage et al. (2014) Nat. Commun. 5,
doi: 10.1038/ncomms5037; Rao et al. (2014) BMC Genomics 15, doi: 10.1186/1471-2164-15-
964). Seed, dark, placenta (plac), leaf, root, and stem tissues are as previously reported (Rao
et al. (2014). Data are in fragments per kilobase per million mapped reads (FPKM).

| | Gallage et al. | Rao et al. | | | | | | |
|---|---|---|---|---|---|---|---|---|
| CPLP gene | 6 m-old pod | 8 w seed | 8 w dark | 5 m seed | 5 m plac | Leaf | Root | Stem |
| Vpl_s027Ag26221 | 0.0 | 34.9 | 37.3 | 58.3 | 32.9 | 41.3 | 46.4 | 28.3 |
| Vpl_s027Bg25947 | 36.7 | 633.6 | 672.0 | 1163.6 | 583.7 | 833.4 | 821.9 | 623.3 |
| Vpl_s027Bg25938 | 210.3 | 229.6 | 256.9 | 460.3 | 281.9 | 298.5 | 307.1 | 229.9 |

In a second study (Yang et al. (2017)), RT-PCR primers overlap three SNPs within CPLP making it difficult to predict amplification specificity as melt curve analysis would not have indicated multiple amplicons as they would be identical among the three CPLP genes. A third study used primers with perfect identity to all three genes in what would have resulted in merged gene transcript abundances across tissues (Fock-Bastide (2014)). A final study used CPLP antibodies (Gallage et al. (2018) Plant and Cell Physiol. 59:304-318) with a C-terminal target that perfectly matched all three predicted protein translations and would not differentiate among alleles or paralogs (FIG. 13C). Resolving each gene independently is important if only certain CPLP alleles or paralogs impact vanillin content in beans.

The identification of multiple CPLP genes and genomics-based transcript levels permitted improved resolution of tissue specificity and the potential role of CPLP in vanillin biosynthesis (FIG. 12C). The haplotype A allele (Vpl_s027Ag26221) had low transcript abundance and was missing 5′ exons in comparison to both haplotype B alleles, which had higher transcript abundance. Haplotype B Vpl_s027Bg25947 showed high transcript abundance across all tissue types, but transcript abundance of Vpl_s027Bg25938 was higher in placental and seed tissues at 5 and 6 months after pollination compared to other tissues (FIG. 12C).

### 1.7 Identifying Candidate Genes for Vanilla Bean Dehiscence

V. planifolia beans split along two abscission zones as they mature towards the peak of quality (Lapeyre-Montes et al. (2010) in Vanilla medicinal and aromatic plants-industrial profiles (ed Eric Odoux and Michel Grisoni) Ch. 10, CRC Press). Seed shattering and pod dehiscence involve common processes leading to seed dispersal in many plant species including Arabidopsis, the Brassicaceae, tomato, soybean, many cereals, and others (Dong & Wang (2015) Front. Plant Sci. 6, doi:10.3389/fpls.201500476). This trait is common in undomesticated crop species resulting in lower agronomic yield. In general, abscission zones are formed during fruit or pod development, and domestication selects against genes that coordinate and form these dehiscence zones. In vanilla, two dehiscence splits open the vanilla beans, but split beans are less desirable at commercial curing facilities (Odoux & Brillouet (2009) Fruits 64:221-241). Domesticated crops often accrue mutations in one or more genes that function in the formation of abscission zones. The sequenced vanilla genome enabled the identification of putative candidate gene variants leading to indehiscent V. x tahitensis beans in comparison to dehiscent V. planifolia beans. Homologs of genes implicated in seed shattering and bean dehiscence from other species were identified in the V. planifolia genome and screened for variants between V. planifolia and V. x tahitensis (Table 8).

TABLE 8
Dehiscence and seed shattering genes from various species, their homologs in V. planifolia, and the number of
homozygous missense variants in V. x tahitensis. Variants are either not present in the V. planifolia accessions
(‘Daphna’, ‘Guy 1’, ‘Hawaii’ and ‘Painter’), or they are present in the heterozygous form. All variants in V. x tahitensis
were missense mutations. Light, dark, placenta, and mesocarp tissues are as previously reported (Rao et al. (2014)
BMC Genomics 15, doi: 10.1186/1471-2164-15-964).

| | | V. planifolia | | V. x. | Expression in FPKM | | | |
|---|---|---|---|---|---|---|---|---|
| | | ‘Daphna’ | Percent | tahitensis | 2 months | | 6 months | |
| Gene | Species | homolog | Identity | variants | Light | Dark | Placenta | Mesocarp |
| SHP1_Shatterproof1 | A. thaliana | Vpl04Ag09199.1 | 62.8 | 0 | 90.7 | 31.9 | 52.5 | 7.7 |
| SHP1_Shatterproof1 | A. thaliana | Vpl06Ag12707.1 | 59.4 | 2 | 2.0 | 9.5 | 3.8 | 1.5 |
| SHP1_Shatterproof1 | A. thaliana | Vpl06Ag12680.1 | 59.4 | 0 | 2.7 | 10.9 | 5.4 | 1.6 |
| IND1_INDEHISCENT | A. thaliana | Vpl09Ag19274.1 | 70.8 | 2 | 0.3 | 0.2 | 0.0 | 0.0 |
| IND1_INDEHISCENT | A. thaliana | Vpl01Ag02046.1 | 78.5 | 0 | 8.4 | 19.4 | 5.3 | 3.1 |
| AL_ALCATRAZ | A. thaliana | Vpl05Ag10812.1 | 79.7 | 0 | 44.0 | 30.4 | 38.1 | 33.2 |
| RPL_REPLUMLESS | A. thaliana | Vpl12Ag22068.1 | 50.1 | 0 | 45.9 | 19.5 | 32.0 | 16.6 |
| RPL_REPLUMLESS | A. thaliana | Vpl01Ag01494.1 | 44.1 | 1 | 17.9 | 37.2 | 17.4 | 41.5 |
| NST1_NAC | A. thaliana | Vpl12Ag22091.1 | 51.5 | 0 | 8.8 | 4.2 | 0.0 | 0.1 |
| NST1_NAC | A. thaliana | Vpl04Ag08909.1 | 48.2 | 4 | 0.0 | 0.0 | 0.0 | 0.0 |
| ADPG1 | A. thaliana | Vpl08Ag17533.1 | 51.5 | 0 | 0.0 | 0.0 | 0.0 | 0.0 |
| ADPG1 | A. thaliana | Vpl06Ag13482.1 | 46.7 | 3 | 0.9 | 0.1 | 0.2 | 0.0 |
| ADPG1 | A. thaliana | Vpl12Ag23164.1 | 45.4 | 0 | 13.0 | 8.5 | 8.7 | 6.7 |
| Dirigent-like protein | G. max | Vpl02Ag05508.1 | 68.0 | 0 | 0.3 | 0.2 | 0.0 | 0.4 |
| OsSh1 | O. sativa | Vpl07Ag14475.1 | 51.4 | 0 | 0.6 | 0.8 | 0.3 | 0.6 |
| OsSh1 | O. sativa | Vpl07Ag14471.1 | 51.4 | 1 | 0.1 | 0.3 | 0.0 | 0.1 |
| OsSh1 | O. sativa | Vpl14Ag25572.1 | 51.7 | 0 | 3.8 | 2.8 | 0.0 | 0.0 |
| OsSh1 | O. sativa | Vpl14Ag25586.1 | 51.7 | 0 | 0.2 | 0.7 | 0.0 | 0.0 |
| OsSh1 | O. sativa | Vpl_s034Ag26329.1 | 53.9 | 0 | 1.8 | 15.3 | 1.4 | 1.5 |
| OsSh1 | O. sativa | Vpl05Ag12156.1 | 52.8 | 0 | 57.1 | 186.2 | 18.2 | 192.6 |
| OsSH4 | O. sativa | Vpl14Ag25846.1 | 57.1 | 0 | 0.0 | 0.1 | 0.0 | 0.0 |
| FUL_AGL8 | A. thaliana | Vpl05Ag12259.1 | 65.5 | 0 | 1.0 | 1.0 | 1.2 | 1.2 |

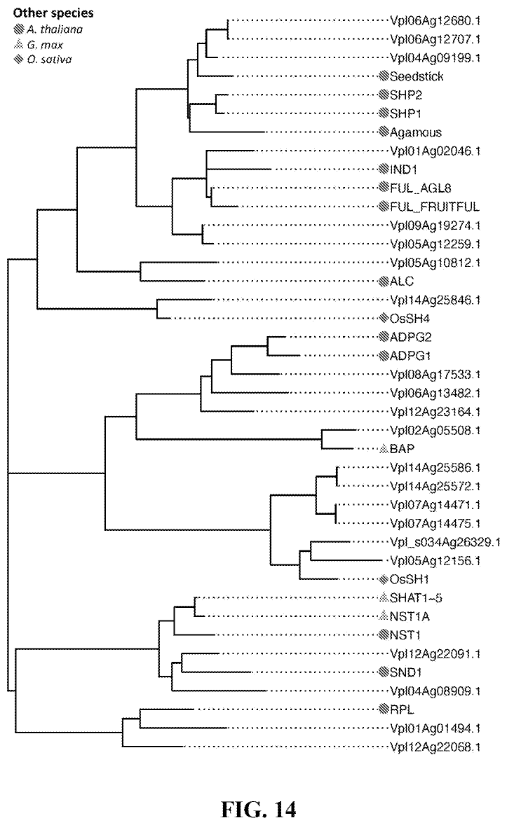

Six of 22 candidate genes (Vpl06Ag12707, Vpl09Ag19274, Vp101Ag01494, Vpl06Ag13482, Vpl07Ag14471, and Vpl04Ag08909) had missense mutations in V. x tahitensis (indehiscent) that were not present in V. planifolia (dehiscent). ‘Daphna’ RNA-seq transcripts from a previous study were used to identify transcript abundance for these six genes during pod development (Rao et al. (2014)). Five out of the six genes (Vpl06Ag12707, Vpl09Ag19274, Vp101Ag01494, Vpl06Ag13482, and Vpl07Ag14471) showed transcript support in developing pods and were similar to the dehiscence-related genes SHATTERPROOF, INDEHISCENT, REPLUMLESS, ADPG1, and OsSH1, respectively, from Arabidopsis and rice (FIG. 14).

These results can now be used to design molecular markers to screen additional accessions and progeny in segregating populations to correlate DNA variants with the indehiscent phenotype. The ability to identify homozygous or heterozygous variants using the phased genome assembly could also guide methods to achieve indehiscent cultivars when selecting breeding parents. This would identify if selfing is sufficient to obtain a homozygous, causal allele, or if hybridization followed by selfing is necessary in order to obtain the desired phenotype.

### 1.8 Identifying MADS-Box Master Regulators

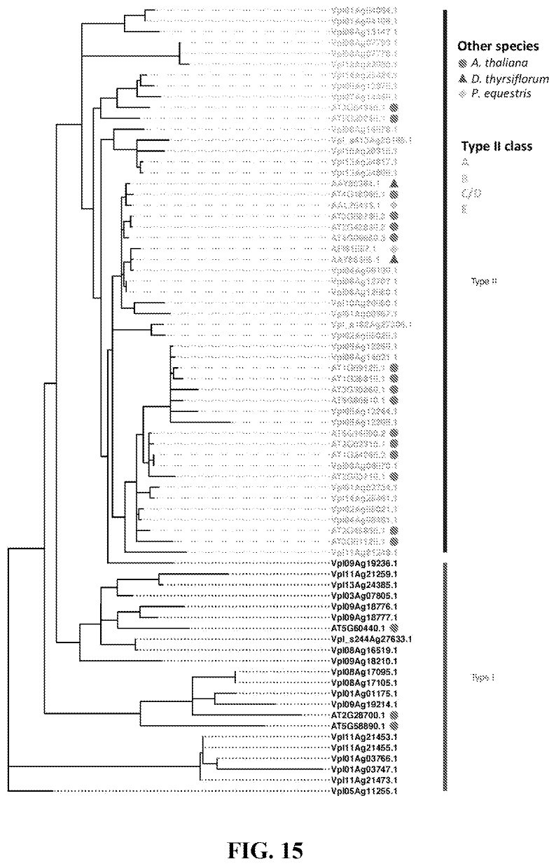

MADS-box transcriptional regulators are central to flower development as part of the ABCDE model where regulators are responsible for initiating specific parts of the flower (sepals, petals, ovules, etc) (Chen et al. (2012) Plant and Cell Physiol. 53:1053-1067). Many vanilla species cannot naturally self pollinate due to the rostellum that physically separates the male and female parts of the flower. Eliminating the rostellum is one route to eliminate the need for manual pollination and remove this tedious and expensive practice in commercial production. Previous work indicated that AGAMOUS, SEEDSTICK, and C- and D-class MADS-box genes were associated with orchid flower development and specifically associated with rostellum tissues in Dendrobium thyrsiflorum and Phaleanopsis equestris (Chen et al. (2012); Skipper et al. (2006) Gene 366:266-274). The V. planifolia genome was screened for master regulators, including various MADS-box genes, that might have an impact on flower and rostellum development (FIG. 15). ‘Daphna’ Vpl04Ag09199 was most similar to PeMADS7 from P. equestris and SEEDSTICK-like from D. thyrsiflorum, though five genes from ‘Daphna’ in total were associated with the clade that included AGAMOUS, SEEDSTICK, and C- and D-class MADS-box from previous orchid studies and Arabidopsis AT4G18960, AT3G58780, and AT2G42830 that are associated with floral organ identity (arabidopsis.org). V. mexicana could also play a role in resolving MADS-box gene function in vanilla flower development. V. mexicana is native to Florida and some Caribbean islands, and has distinct morphological characteristics including the absence of a rostellum. Further analysis of this trait and the impact of MADS-box genes on plant development in vanilla will require the development of an efficient vanilla transformation protocol and dedicated studies to dissect this trait in vanilla.

### 1.9 Cataloguing Disease Resistance Genes

Plant disease resistance genes naturally protect plant health and are critical for defense against plant pathogens. Differences in disease resistance within and among species can be used to identify causal genetic variants that impart disease resistance. For example, V. planifolia is susceptible to the fungal pathogen F. oxysporum f. sp. vanilla, but V. pompona is resistant and could be a genetic donor for Fusarium disease resistance in hybrids (Childers (1948) “Vanilla culture in Puerto Rico” US Department of Agriculture 28; Belanger & Havkin-Frenkel (2011) Handbook of Vanilla Science and Technology, Ch. 15, Wiley). The availability of a genome sequence facilitates the efficient identification of resistance genes even in complex heterozygous genomes (Li et al. (2016) BMC Genomics 17, doi:10.1186/s12864-016-3197-x; Rody et al. (2019) BMC Genomics 20, doi:10.1186/s12864-019-6207-y). Cataloging annotated resistance gene analogs (RGA) in the annotated ‘Daphna’ genome identified 1,102 and 1,119 RGAs in haplotypes A and B, respectively (Tables 9 and 10).

TABLE 9
List of identified RGA (Resistance Gene Analog) genes. The
majority of RGAs belong to three different classes: Kinases
(KIN) characterized by intracellular kinase-like and transmembrane
domains; Receptor Like Kinases (RLK) characterized by an intracellular
kinase-like domain, an extracellular leucine- rich repeat domain,
and a transmembrane domain; and Receptor Like Proteins (RLP) that
are similar to RLKs but lack the kinase-like domain. Shown are all
identified RGAs in V. planifolia ‘Daphna’ genome
for both haplotypes.

| Class | Domains | Haplotype A | Haplotype B |
|---|---|---|---|
| KIN | Kin + TM | 642 | 663 |
| RLK | LRR + Kin + TM | 155 | 147 |
| RLP | TM + LRR | 116 | 122 |
| CK | CC + Kin + TM | 64 | 65 |
| N | NBS + TM | 64 | 60 |
| L | LRR | 23 | 21 |
| CN | CC + NBS + TM | 11 | 14 |
| CNL | CC + NBS + LRR + TM | 8 | 7 |
| CLK | CC + LRR + Kin + TM | 6 | 5 |
| NL | NBS + LRR + TM | 3 | 1 |
| TRAN | TM | 3 | 3 |
| T | TIR + TM | 2 | 2 |
| CL | CC + LRR + TM | 2 | 6 |
| C | CC + TM | 2 | 2 |
| NK | NBS + Kin + TM | 1 | 1 |
| Total | | 1102 | 1119 |

CC: Coiled Coil,
Kin: Kinase,
LRR: Leucine rich region,
NBS: nucleotide binding site,
TIR: Toll-interleukin region,
TM: transmembrane

TABLE 10
Resistance gene analogs (RGAs) identified in the V. planifolia genome. Included are RGA variants identified in V.
pompona and transcript support from a previous study (Rao et al. (2014) BMC Genomics 15, doi: 10.1186/1471-2164-
15-964). Light, dark, mesocarp, placenta, leaf, root, and stem tissues are as previously reported (Rao et al. (2014)).

| | | | | Expression (FPKM) | | | | | | |
|---|---|---|---|---|---|---|---|---|---|---|
| | RGA | Variants in V. pompona | | 8 weeks | | 6 months | | Young | | |
| Gene | class | Moderate¹ | High² | Light | Dark | Meso | Plac | Leaf | Root | Stem |
| Vpl01Ag02213.1 | CNL | 0 | 0 | 857.5 | 148.3 | 87.7 | 883.6 | 18.1 | 225.8 | 71.3 |
| Vpl02Ag05172.1 | CNL | 24 | 1 | 1.8 | 1.7 | 2.2 | 0.8 | 5.4 | 15.3 | 33.3 |
| Vpl03Ag07482.1 | NL | 0 | 0 | 4.2 | 3.9 | 3.7 | 3.0 | 1.9 | 3.3 | 2.8 |
| Vpl04Ag08516.1 | NL | 0 | 0 | 0.0 | 0.0 | 0.0 | 0.0 | 0.0 | 0.0 | 0.0 |
| Vpl04Ag08520.1 | CNL | 0 | 0 | 0.0 | 0.0 | 0.0 | 0.0 | 0.0 | 0.0 | 0.0 |
| Vpl04Ag08522.1 | CNL | 0 | 0 | 0.2 | 0.2 | 0.1 | 0.0 | 0.1 | 0.1 | 0.0 |
| Vpl04Ag08525.1 | NL | 0 | 0 | 0.7 | 0.3 | 0.1 | 0.2 | 0.4 | 10.2 | 0.2 |
| Vpl06Ag14231.1 | CNL | 0 | 0 | 74.3 | 45.7 | 26.1 | 22.8 | 20.8 | 33.1 | 24.3 |
| Vpl14Ag25867.1 | CNL | 17 | 0 | 28.2 | 28.2 | 42.6 | 32.4 | 29.5 | 45.6 | 35.7 |
| Vpl_s049Ag26492 | CNL | 0 | 0 | 21.6 | 11.1 | 6.6 | 8.7 | 5.3 | 9.3 | 6.2 |
| Vpl_s056Ag26537 | CNL | 12 | 0 | 9.5 | 2.3 | 1.2 | 5.5 | 1.8 | 11.1 | 3.3 |

¹Moderate impact as defined by snpEff.
²High impact as defined by snpEff.

Further, a motif search (NBS, LRR, TM) using protein sequences of known Fusarium resistance genes from other plant species identified eleven putative RGA proteins in V. planifolia (Table 10). Of the eleven putative RGAs, nine had transcript support in V. planifolia roots, and three (Vpl02Ag05172.1, Vpl14Ag25867.1, and Vpl_s056Ag26537.1) also had sequence variants in V. pompona that could be responsible for conferring resistance against Fusarium. Ongoing research with segregating populations will aid in identifying the genetic factors controlling Fusarium resistance in V. pompona which will be fundamental to integrating this trait into the commercial species, V. planifolia.

Table 11 provides information regarding all the accessions used in these studies.

TABLE 11
List of accessions in this study including species, accession name, and accession numbers from Hu
et al (2019) Sci. Rep. 9: 3416). Reads (in millions), bases sequenced (Gb), GC content (%), accession
origin, and breeding utility are shown.

| | | | Reads (in | Bases | | | |
|---|---|---|---|---|---|---|---|
| Species | Name | Accession # | millions) | (Gb) | GC Content (%) | Origin | Breeding utility |
| V. planifolia | ‘Daphna’ | AC173 | 1,182.10 | 177.3 | 31.4 | Madagascar | Accession represents |
| | | | | | | | common Madagascar |
| | | | | | | | commercial clones. |
| V. planifolia | ‘Guy 1’ | AC184 | 559.2 | 81.3 | 32.8 | Hawaii, USA | Bean-producing accession in |
| | | | | | | | commercial production. |
| V. planifolia | ‘Hawaii’ | AC199 | 586.3 | 85.0 | 33.2 | Hawaii, USA | Kadooka's vanilla, this clone |
| | | | | | | | represents vanilla on its |
| | | | | | | | trans-Pacific migratory |
| | | | | | | | route. |
| V. planifolia | ‘Painter’ | AC198 | 595.8 | 85.1 | 33.0 | Florida, USA | Clone produces long (~21 |
| | | | | | | | cm) beans and is adapted to |
| | | | | | | | Florida. |
| V. pompona | ‘King’ | AC200 | 515.3 | 73.7 | 33.9 | Florida, USA | Fusarium disease-resistant |
| | | | | | | | species that is adapted to |
| | | | | | | | Florida. |
| V. x tahitensis | ‘Haapape’ | — | 580.0 | 84.4 | 33.5 | Tahiti | Commercial clone with |
| | | | | | | | distinct aroma and non- |
| | | | | | | | splitting beans. |
| V. mexicana | ‘Sheila’ | AC191 | 749.5 | 108.3 | 36.9 | Florida, USA | Florida native species that is |
| | | | | | | | genetically distant from V. |
| | | | | | | | planifolia and lacks a |
| | | | | | | | rostellum. |

### Example 2. Increased Vanillin Biosynthesis Through Overexpression of Phenylalanine Ammonia Lyase (PAL)

Phenylalanine ammonia lyase (PAL) is the first enzyme of the phenylpropanoid pathway in the proposed route for vanillin bio-synthesis. As described in Example 1, four PAL homologs were identified in the genome assembly, two in each haplotype: Vpl_s453Bg28354, Vpl03Ag07441, Vpl03Ag07445, and Vpl03Bg07223. Only the expression of Vpl_s453Bg28354 was detected in the developing pod tissues of the Vanilla planifolia cultivar ‘Daphna’. This allele may be responsible for generating most of the influx in the vanillin pathway. The goal of these studies is to create vanilla accessions with higher vanillin content. One method of doing so is by increasing the influx to the vanillin pathway by creating homozygous accessions with the PAL allele Vpl_s453Bg28354. In this method, genome assembly is used to create markers specific to the Vpl_s453Bg28354 allele and a conventional breeding approach is used by self-pollinating V. planifolia cultivar ‘Daphna’. If inbreeding depression is a concern, multiple accessions of V. planifolia are screened with markers for Vpl_s453Bg28354 and crosses are made based on marker segregation results. Markers are then used to screen F1 for homozygous individuals.

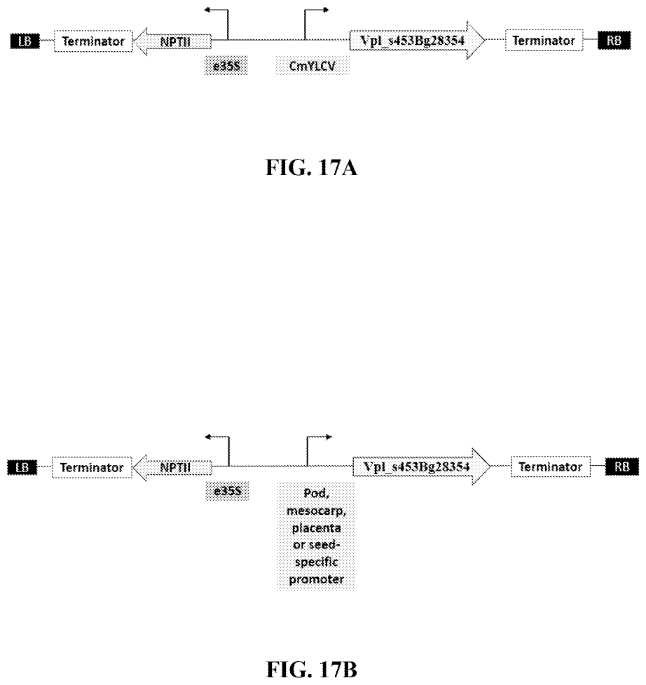

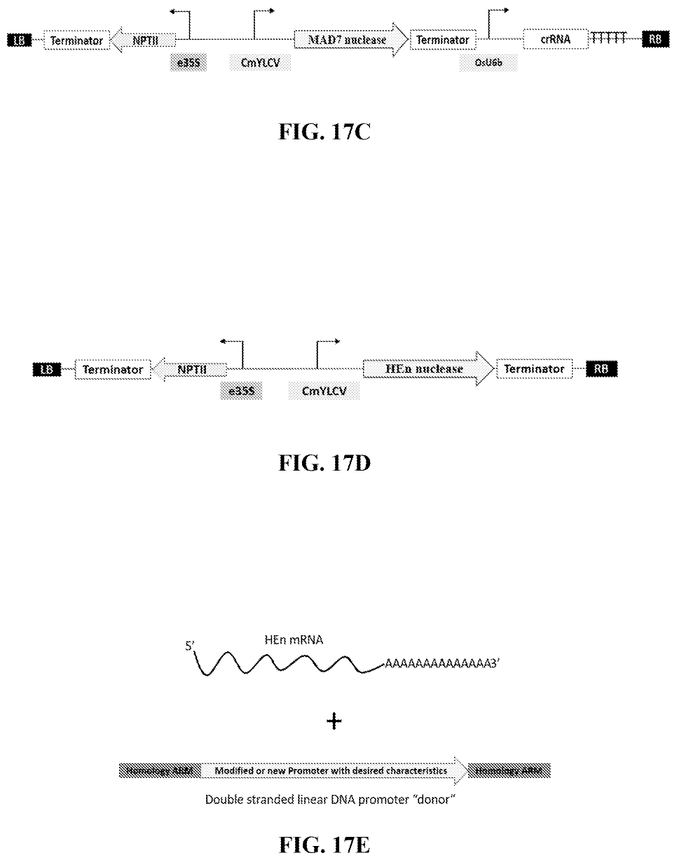

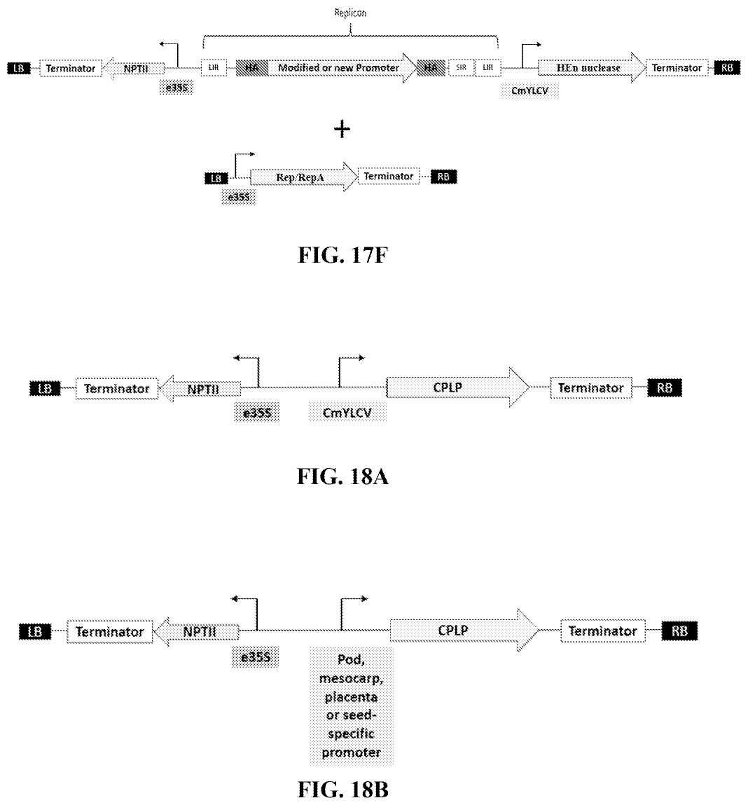

Another method of increasing vanillin content is by using a transgenic/genome editing approach to increase the expression of Vpl_s453Bg28354. One such approach, outlined in FIG. 17A, involves Agrobacterium-mediated transformation of V. planifolia using a binary payload containing the neomycin phosphotransferase II (NPTII) gene driven by a strong, viral constitutive promoter in monocots, e.g., 35S, as well as an expression cassette containing the Vpl_s453Bg28354 allele driving by another strong, viral constitutive promoter, e.g., the Cestrum yellow leaf curling virus (CmYLCV) promoter. As a variation to this approach, expression of the Vpl_s453Bg28354 allele is restricted to the vanilla pod or sub-tissues, e.g., mesocarp, placenta, hair cells, seeds. This is done using RNAseq data and identifying genes in addition to PAL exhibiting this tissue restriction, but high expression, and these promoters are used in a very similar payload, as exemplified in FIG. 17B.

In another approach outlined in FIG. 17C, gene editing is used to modify the sequence of the Vpl_s453Bg28354 allele promoter in such a way that results in increased expression. In one instance, this is done using the MAD7 nuclease. V. planifolia are transformed by Agrobacterium using a binary payload containing the NPTII gene driven by a strong, viral constitutive promoter in monocots, e.g., 355, as well as an expression cassette containing the MAD7 nuclease driven by another strong, viral constitutive promoter, e.g., CmYLCV. The payload also contains a cassette expressing a crRNA specific for targeting MAD7 to a region in the Vpl_s453Bg28354 allele promoter. This is driven by the constitutive rice Pol III promoter, OsU6b. Similarly, the Vpl_s453Bg28354 allele promoter is targeted using engineered homing endonucleases (HEn) using a similar construct. In this case shown in FIG. 17D, the MAD7 nuclease is exchanged for a HEn nuclease and the crRNA would no longer be needed.

In another gene editing approach, the endogenous Vpl_s453Bg28354 allele promoter is exchanged with a modified version that has higher expression levels. This is due to an exchange with an entirely transgenic promoter or a pre-modified variant of the Vpl_s453Bg28354 promoter. This sort of homology-directed recombination (HDR) shown in FIG. 17E can be achieved at the protoplast level using mRNA for either MAD7 or HEn targeting the 5′ and 3′ boundaries of the native promoter while adding a double stranded donor DNA for the targeted exchange. Additionally, the same is achieved in V. planifolia tissue using a geminiviral payload in a binary backbone. In this case illustrated in FIG. 17F, the binary payload contains the NPTII selection marker and one or more HEn nuclease(s) targeting the DNA flanking the native Vpl_s453Bg28354 allele promoter. Between the long inverted repeats (LIR) of the Geminivirus (e.g., wheat dwarf virus), is the modified or transgenic promoter with homology arm sequences identical to the flanking sequences of the Vpl_s453Bg28354 allele promoter. Delivery of this payload via Agrobacterium in trans with an additional payload consisting of just the Geminiviral Rep/RepA protein results in the replacement of the original Vpl_s453Bg28354 allele promoter with the intended modified or transgenic version.

### Example 3. Increased Vanillin Biosynthesis Through Overexpression of Cysteine Protease-Like Protein (CPLP)

Cysteine protease-like protein (CPLP) is a critical enzyme in the proposed route for vanillin bio-synthesis. As described in Example 1, three CPLP homologs were identified in the genome assembly: Vpl_s027Ag26221, Vpls027Bg25938, and Vpls027Bg25947. Expression of Vpl_s027Bg25947 and Vpl_s027Bg25938 was detected in the developing pod tissues of the Vanilla planifolia cultivar ‘Daphna’. These alleles may be responsible for generating much of the influx in the vanillin pathway. The goal of these studies is to create vanilla accessions with higher vanillin content. One method of doing so is by increasing the influx to the vanillin pathway by creating homozygous accessions with the CPLP allele Vpl_s027Bg25947 and/or Vpl_s027Bg25938. In this method, genome assembly is used to create markers specific to the Vpl_s027Bg25947 and Vpl_s027Bg25938 alleles and a conventional breeding approach is used by self-pollinating V. planifolia cultivar ‘Daphna’. If inbreeding depression is a concern, multiple accessions of V. planifolia are screened with markers for Vpl_s027Bg25947 and/or Vpl_s027Bg25938 and crosses are made based on marker segregation results. Markers are then used to screen F1 for homozygous individuals.

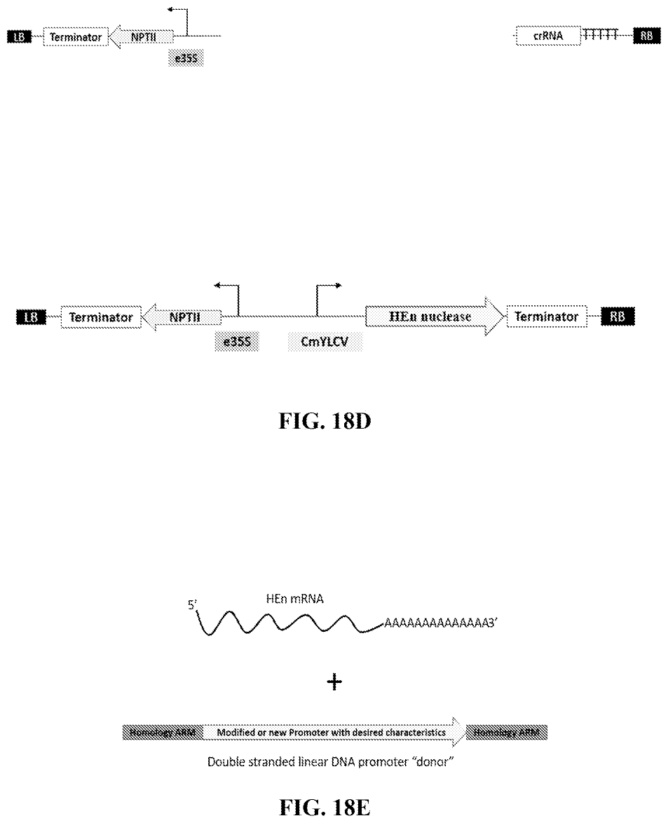

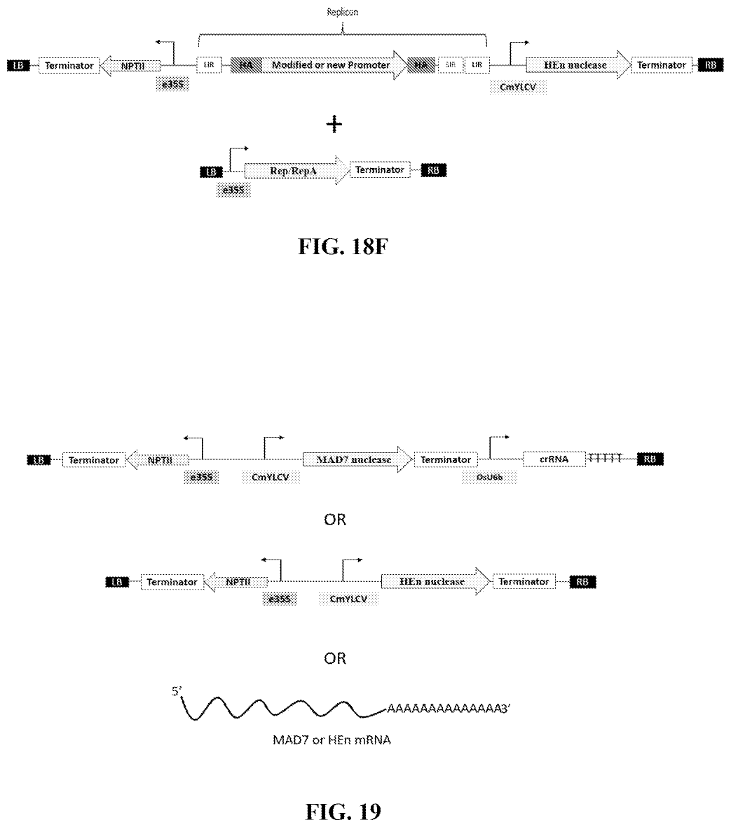

Another method of increasing vanillin content is by using a transgenic/genome editing approach to increase the expression of Vpl_s027Bg25947 and/or Vpl_s027Bg25938. One such approach, outlined in FIG. 18A, involves Agrobacterium-mediated transformation of V. planifolia using a binary payload containing the neomycin phosphotransferase II (NPTII) gene driven by a strong, viral constitutive promoter in monocots, e.g., 35S, as well as an expression cassette containing the Vpl_s027Bg25947 and/or Vpl_s027Bg25938 allele driving by another strong, viral constitutive promoter, e.g., the Cestrum yellow leaf curling virus (CmYLCV) promoter. As a variation to this approach, expression of the Vpl_s027Bg25947 and/or Vpl_s027Bg25938 allele is restricted to the vanilla pod or sub-tissues, e.g., mesocarp, placenta, hair cells, seeds. This is done using RNAseq data and identifying genes exhibiting this tissue restriction, but high expression, and these promoters are used in a very similar payload, as exemplified in FIG. 18B.

In another approach outlined in FIG. 18C, gene editing is used to modify the sequence of the Vpl_s027Bg25947 and/or Vpl_s027Bg25938 allele promoter in such a way that results in increased expression. In one instance, this is done using the MAD7 nuclease. V. planifolia are transformed by Agrobacterium using a binary payload containing the NPTII gene driven by a strong, viral constitutive promoter in monocots, e.g., 355, as well as an expression cassette containing the MAD7 nuclease driven by another strong, viral constitutive promoter, e.g., CmYLCV. The payload also contains a cassette expressing a crRNA specific for targeting MAD7 to a region in the Vpl_s027Bg25947 and/or Vpl_s027Bg25938 allele promoter. This is driven by the constitutive rice Pol III promoter, OsU6b. Similarly, the Vpl_s027Bg25947 and/or Vpl_s027Bg25938 allele promoter is targeted using engineered homing endonucleases (HEn) using a similar construct. In this case shown in FIG. 18D, the MAD7 nuclease is exchanged for a HEn nuclease and the crRNA would no longer be needed.

In another gene editing approach, the endogenous Vpl_s027Bg25947 and/or Vpl_s027Bg25938 allele promoter is exchanged with a modified version that has higher expression levels. This is due to an exchange with an entirely transgenic promoter or a pre-modified variant of the Vpl_s027Bg25947 and/or Vpl_s027Bg25938 promoter. This sort of homology-directed recombination (HDR) shown in FIG. 18E can be achieved at the protoplast level using mRNA for either MAD7 or HEn targeting the 5′ and 3′ boundaries of the native promoter while adding a double stranded donor DNA for the targeted exchange. Additionally, the same is achieved in V. planifolia tissue using a geminiviral payload in a binary backbone. In this case illustrated in FIG. 18F, the binary payload contains the NPTII selection marker and one or more HEn nuclease(s) targeting the DNA flanking the native Vpl_s027Bg25947 and/or Vpl_s027Bg25938 allele promoter. Between the long inverted repeats (LIR) of the Geminivirus (e.g., wheat dwarf virus), is the modified or transgenic promoter with homology arm sequences identical to the flanking sequences of the Vpl_s027Bg25947 and/or Vpl_s027Bg25938 allele promoter. Delivery of this payload via Agrobacterium in trans with an additional payload consisting of just the Geminiviral Rep/RepA protein results in the replacement of the original Vpl_s027Bg25947 and/or Vpl_s027Bg25938 allele promoter with the intended modified or transgenic version.

### Example 4. Reduced Pod Shattering by Disrupting Indehiscent Genes

V. planifolia pods are likely to split at maturity, causing yield loss. Example 1 describes the identification of vanilla homologs of known dehiscence and seed shattering genes from other species. The following homologs contain missense mutations in indehiscent V. x. tahitensis compared to dehiscent V. planifolia ‘Daphna’. These genes are also expressed in pods during development: Vpl06Ag12707.1 (SHATTERPROOF), Vpl09Ag19274.1 (INDEHISCENT), Vp101Ag01494.1 (REPLUMLESS), Vpl06Ag13482.1 (ADPG1), and Vpl07Ag14471.1 (OsSH1). The goal of these studies is to create vanilla accessions with indehiscent beans by modifying abscission tissues in vanilla pods by creating individual knockouts for each of the five candidate genes listed above. Appropriate MAD7 or HEn target sequences are identified that should be expected, after editing, to result in a frame shift or nonsense mutation, disrupting normal gene function. This is obtained in transgenic plants via Agrobacterium transformation of V. planifolia using binary payloads containing either MAD7 targeted to the gene of interest or a HEn nuclease. Alternatively, transgenic plants are achieved by transfection of protoplasts with mRNA coding for the MAD7 or HEn nuclease, followed by regeneration of plants from protoplasts. These methods are outlined in FIG. 19.

### Example 5. Self-Pollination of Vanilla Plants by Disrupting MADS-Box Genes

Vanilla flowers must be hand pollinated to circumvent the rostellum that physically separates the male and female parts of the flower. AGAMOUS, SEEDSTICK, and C- and D-class MADS-box genes are associated with orchid flower development, and specifically with rostellum tissues in D. thyrsiflorum and P. equestris. Five genes were identified in Example 1 from V. planifolia ‘Daphna’ that are associated with the clade that includes AGAMOUS and SEEDSTICK from D. thyrsiflorum and P. equestris, as well as other C- and D-class MADS-box genes from Arabidopsis: Vpl04Ag09199.1, Vpl06Ag12707.1, Vpl06Ag12680.1, Vp110Ag20060.1, and Vp101Ag00567.1. In particular, Vpl04Ag09199.1 is most similar to PeMADS7 from P. equestris and SEEDSTICK-like from D. thyrsiflorum. The goal of these studies is to create self-pollinating accessions of vanilla that require no intervention from humans or natural pollinators by removing or modifying the vanilla flower's rostellum by creating individual knockouts for each of the five candidate MADS-box genes listed above. Appropriate MAD7 or HEn target sequences are identified that should be expected, after editing, to result in a frame shift or nonsense mutation, disrupting normal gene function. This is obtained in transgenic plants via Agrobacterium transformation of V. planifolia using binary payloads containing either MAD7 targeted to the gene of interest or a HEn nuclease. Alternatively, transgenic plants are achieved by transfection of protoplasts with mRNA coding for the MAD7 or HEn nuclease, followed by regeneration of plants from protoplasts. These methods are outlined in FIG. 19.

The contents of all references, patents, pending patent applications, and publications cited throughout this application are hereby expressly incorporated by reference herein in their entirety.

Those skilled in the art will recognize, or be able to ascertain using no more than routine experimentation, many equivalents to the specific embodiments of the invention described herein. Such equivalents are intended to be encompassed by the following claims.

1. A method for producing a Vanilla sp. plant having improved flavor or agronomic production, comprising:
a) increasing expression of a phenylalanine ammonia lyase (PAL) gene in said Vanilla sp. plant;
b) increasing expression of a cysteine protease-like protein (CPLP) gene in said Vanilla sp. plant;
c) reducing dehiscence in said Vanilla sp. plant;
d) reducing expression of at least one MADS-box gene in said Vanilla sp. plant; or
e) increasing fungal resistance in said Vanilla sp. plant.

2. The method of claim 1, wherein increasing expression of said PAL gene comprises:
a) introducing into said Vanilla sp. plant a nucleic acid molecule comprising a nucleotide sequence encoding a PAL polypeptide;
b) genetically-modifying an endogenous promoter of said PAL gene; or
c) genetically-modifying the genome of said Vanilla sp. plant to comprise at least two copies of said PAL gene.

3. The method of claim 1, wherein:
a) said PAL gene encodes a polypeptide having an amino acid sequence having at least 95% sequence identity to SEQ ID NO: 1 or 2; or
b) said Vanilla sp. plant produces increased levels of cinnamic acid or vanillin, or one or more precursors of vanillin.

4. (canceled)

5. The method of claim 1, wherein increasing expression of said CPLP gene comprises:
a) introducing into said Vanilla sp. plant a nucleic acid molecule comprising a nucleotide sequence encoding a CPLP polypeptide;
b) genetically-modifying an endogenous promoter of said CPLP gene; or
c) genetically-modifying the genome of said Vanilla sp. plant to comprise at least two copies of said CPLP gene.

6. (canceled)

7. The method of claim 5, wherein:
a) said CPLP gene encodes a CPLP transcript comprising exons 1-3;
b) said CPLP gene encodes an amino acid sequence having at least 95% sequence identity to SEQ ID NO: 9 or 11;
c) said CPLP gene encodes amino acid residues 1-144, and a serine at a position corresponding to 151 of SEQ ID NO: 9 or 11;
d) said CPLP gene has at least 95% sequence identity to SEQ ID NO: 10 or 12 or
e) said Vanilla sp. plant produces increased levels of 4-hydroxybenzaldehyde or vanillin, or one or more precursors of vanillin.

8. (canceled)

9. (canceled)

10. (canceled)

11. (canceled)

12. The method of claim 1, wherein said Vanilla sp. plant is a Vanilla planifolia plant, a Vanilla x tahitensis plant, a Vanilla odorata plant, or a Vanilla pompona plant.

13. (canceled)

14. (canceled)

15. A genetically-modified Vanilla sp. plant comprising:
a) increased expression of a phenylalanine ammonia lyase (PAL) gene;
b) increased expression of a cysteine protease-like protein (CPLP) gene;
c) reduced dehiscence;
d) reduced expression of at least one MADS-box gene; or
e) increased fungal resistance,
as compared to a non-genetically-modified Vanilla sp. plant.

16. The genetically-modified Vanilla sp. plant of claim 15, wherein:
a) a nucleic acid molecule comprising a nucleotide sequence encoding a PAL polypeptide has been stably integrated into the genome of the Vanilla sp. plant;
b) an endogenous promoter of said PAL gene has been genetically-modified to increase expression of said PAL gene; or
c) said Vanilla sp. plant comprises said at least two copies of said PAL gene.

17. (canceled)

18. (canceled)

19. (canceled)

20. (canceled)

21. The genetically-modified Vanilla sp. plant of claim 15, wherein:
a) a nucleic acid molecule comprising a nucleotide sequence encoding a CPLP polypeptide has been stably integrated into the genome of the Vanilla sp. plant;
b) an endogenous promoter of said CPLP gene has been genetically-modified to increase expression of said CPLP gene; or
c) said genetically-modified Vanilla sp. plant comprises at least two copies of said CPLP gene.

22. (canceled)

23. (canceled)

24. (canceled)

25. (canceled)

26. (canceled)

27. (canceled)

28. (canceled)

29. (canceled)

30. A Vanilla sp. plant part of the genetically-modified Vanilla sp. plant of claim 15.

31. (canceled)

32. The Vanilla sp. plant part of claim 30, wherein said plant part comprises a bean, a seed, a seed a seed capsule or a cell.

33. The method of claim 1, comprising:
a) introducing at least one indehiscence-associated mutation into at least one dehiscent gene in said Vanilla sp. plant;
b) reducing the expression of at least one dehiscent gene in said Vanilla sp. plant; or
c) at least one copy of at least one dehiscent gene is disrupted or knocked out,
wherein said dehiscent gene encodes a Shatterproof, Indehiscent, Replumless, Adpg1, or Sh1 protein.

34. (canceled)

35. (canceled)

36. (canceled)

37. The method of claim 33, wherein said at least one indehiscence-associated mutation into at least one dehiscent gene:
a) reduces the activity of the encoded protein; or
b) introduces at least one missense mutation or at least one nonsense mutation such that said dehiscent gene encodes a truncated protein.

38. (canceled)

39. The method of claim 33, wherein said dehiscent gene encodes:
a) a Shatterproof protein, wherein said indehiscence-associated mutation is selected from one that results in at least one of:
i) a leucine at a position corresponding to 149 of SEQ ID NO: 15; and
ii) a tyrosine at a position corresponding to 165 of SEQ ID NO: 15;
b) an Indehiscent protein, wherein said indehiscence-associated mutation is selected from one that results in at least one of:
i) a serine inserted in between positions corresponding to 45 and 46 of SEQ ID NO: 17; and
ii) a proline at a position corresponding to 35 of SEQ ID NO: 17;
c) a Replumless protein, wherein said indehiscence-associated mutation results in a glycine at a position corresponding to 10 of SEQ ID NO: 19;
d) an Adpg1 protein, wherein said indehiscence-associated mutation is selected from one that results in at least one of:
i) a tryptophan at a position corresponding to 29 of SEQ ID NO: 21;
ii) a serine at a position corresponding to 15 of SEQ ID NO: 21; and
iii) an aspartic acid at a position corresponding to 12 of SEQ ID NO: 21; or
e) a Sh1 protein, wherein said indehiscence-associated mutation results in a threonine at a position corresponding to 113 of SEQ ID NO: 23.

40. (canceled)

41. (canceled)

42. The genetically-modified Vanilla sp. plant of claim 15, comprising:
a) at least one indehiscence-associated mutation in at least one dehiscent gene; or
b) reduced expression of at least one dehiscent gene
wherein said dehiscent gene encodes a Shatterproof, Indehiscent, Replumless, Adpg1, or Sh1 protein.

43. (canceled)

44. (canceled)

45. (canceled)

46. (canceled)

47. (canceled)

48. (canceled)

49. (canceled)

50. (canceled)

51. (canceled)

52. The method of claim 1, wherein reducing the expression of said at least one MADS-box gene in said Vanilla sp. plant comprises genetically-modifying the genome of said Vanilla sp. plant to reduce the expression of said at least one MADS-box gene to generate a genetically-modified Vanilla sp. plant cell.

53. The method of claim 52, wherein said MADS-box gene encodes a MADS-box protein comprising a sequence selected from the group consisting of SEQ ID NOs: 26, 28, 30, 32, and 34.

54. The method of claim 52, wherein genetically-modifying the Vanilla sp. genome comprises:
a) disrupting or knocking out at least one copy of at least one MADS-box gene;
b) disrupting or knocking out all copies of at least one MADS-box gene: or
c) mutating at least one MADS-box gene such that the activity of the encoded protein is reduced.

55. (canceled)

56. (canceled)

57. The method of claim 54, wherein mutating said gene comprises introducing at least one missense mutation or at least one nonsense mutation such that said MADS-box gene encodes a truncated protein.

58. (canceled)

59. (canceled)

60. (canceled)

61. The genetically-modified Vanilla sp. plant of claim 15, wherein the genome of the genetically-modified Vanilla sp. plant comprises at least one genetic modification that reduces the expression of at least one gene encoding a MADS-box protein compared to a non-genetically-modified Vanilla sp. plant cell.

62. (canceled)

63. (canceled)

64. (canceled)

65. (canceled)

66. (canceled)

67. (canceled)

68. (canceled)

69. The Vanilla sp. plant of claim 61, wherein said Vanilla sp. plant has a flower comprising a rostellum of reduced size compared to a control plant not comprising said genetic modification or wherein said Vanilla sp. plant lacks a rostellum.

70. The Vanilla sp. plant of claim 69, wherein said genetically-modified Vanilla sp. plant is capable of self-pollination.

71. The method of claim 1, said method comprising genetically-modifying the genome of said Vanilla sp. plant to introduce at least one pompona-associated mutation within at least one endogenous inactive fungal resistance gene such that the introduction of said at least one pompona-associated mutation in said endogenous inactive fungal resistance gene generates an active fungal resistance gene that encodes a fungal resistance protein.

72. (canceled)

73. The method of claim 71, wherein said inactive fungal resistance gene encodes a fungal resistance protein that has an amino acid sequence having at least 95% sequence identity to any one of SEQ ID NOs: 36, 38, and 40.

74. The method of claim 73, wherein said inactive fungal resistance protein is mutated to comprise at least one of the amino acid residues selected from the group consisting of:
a) any one of a glycine, glutamic acid, histidine, glutamic acid, threonine, serine, lysine, histidine, leucine, isoleucine, glycine, arginine, leucine, aspartic acid, aspartic acid, glycine, asparagine, methionine, methionine, aspartic acid, glutamine, aspartic acid, asparagine, alanine, and glycine at positions corresponding to 28, 82, 91, 113, 131, 132, 147, 193, 199, 207, 227, 246, 271, 318, 324, 333, 336, 367, 379, 380, 408, 433, 443, 460, and 462, respectively of SEQ ID NO: 36;
b) any one of a glutamic acid, aspartic acid, glycine, methionine, methionine, threonine, isoleucine, lysine, arginine, lysine, asparagine, phenylalanine, lysine, proline, phenylalanine, lysine, and alanine at positions corresponding to 29, 107, 216, 229, 362, 404, 547, 574, 610, 638, 695, 706, 773, 840, 860, 870, and 889, respectively of SEQ ID NO: 38; and
c) any one of an alanine, glycine, isoleucine, glutamic acid, alanine, serine, tyrosine, methionine, lysine, glutamine, lysine, and serine at positions corresponding to 91, 124, 227, 333, 381, 537, 555, 703, 716, 754, 758, and 768, respectively of SEQ ID NO: 40.

75. (canceled)

76. The method of claim 71, wherein said fungus is a Fusarium sp.

77. The Vanilla sp. plant of claim 15, wherein the genome of the Vanilla sp. plant is genetically-modified to comprise a pompona-associated mutation within at least one endogenous fungal resistance gene.

78. (canceled)

79. (canceled)

80. (canceled)

81. (canceled)

82. (canceled)

83. (canceled)

84. An extract from the Vanilla sp. plant or plant part of claim 30.


SEQUENCE LISTING (41 sequences)

SEQ ID NO: 1 - protein, 712 aa, Vanilla planifolia
  MISC_FEATURE (1)..(712): Vpl_s453Bg28354 allele gene product
  MISC_FEATURE (1)..(712): Vanilla planifolia ′Daphna′
MEHSKQNGHANGIVEGLCIPTPVRDPLNWGVAAAGMQGSHLEEVKRMVEEFRRPVVRLEGAELKISQVAAVAAGAVGLVELEEAARAGVKASSEWVLDSMNRGTDSYGVTTGFGATSHRRTKQGGALQKELIRFLNAGIFGSGTQNTLPCPVARAAMLVRINTLLQGYSGIRFEILEAITSLINSNITPCLPTRGTITASGDLVPLSYIAGILTGRPNAKAVTAEGEIVDASEAFRLAGIPDGFFELQPKEGLALVNGTAVGSGLASIVLFDANILTVMAVVLSALFCEVMQGKPEYTDHLTHKLKHHPGQIEAAAIMEHILEGSSYMKMAKKLHEMDPLQKPKQDRYALRTSPQWLGPQIEVIRAANKSIEREINSVNDNPLIDVSRNKALHGGNFQGTPIGVSMDNTRLAVAAIGKLMFAQFSELVNDFYNNGLPSNLSGGRNPSLDYGFKGAEIAMASYCSELQALANPVTNHVQSAEQHNQDVNSLGLISARKTAEAVEILKLMTTTFLVGLCQAVDLRHLEENLKFAVKNTVSQVAKRILTMGVNGELHPSRFCEKDLLNVIEREYVFAYADDPCNSVYPLMPKLRQVLVEHAINNGEKEKDPNTSIFQKITAFEEELKMALPKEVEAVRVAFEMGSSAIENRIRDCRSYPLYLFVKEVGAGFLSGDKTISPGEEFDKVFIAISKGKAIDPLLKCLQDWNGAPLPIC

SEQ ID NO: 2 - DNA, 2139 bp, Vanilla planifolia
  misc_feature (1)..(2139): Vpl_s453Bg28354 allele
  misc_feature (1)..(2139): Vanilla planifolia ′Daphna′
atggagcattctaagcagaacgggcacgcgaatgggatcgtggaggggctgtgtattccgaccccggtgagggatccactaaactggggagtggcggcggcggggatgcaaggaagccaccttgaggaggtgaagaggatggtggaggagttccgacgtccggtggtgagactcgagggagcggagctgaagatatctcaggtggcagcggtggccgccggcgccgtcggtcttgtcgagctcgaagaggccgcccgcgctggggtaaaggccagcagtgagtgggttctcgatagcatgaaccgaggaactgacagctatggggtaaccaccggcttcggggccacctcccaccgccgtaccaagcagggcggcgccttgcagaaggagctcatcagattcctcaatgctgggattttcggctctgggacgcagaacaccctgccttgtccggtggcaagggcggcgatgctcgtcagaatcaacactctgcttcagggctactccggcatccgcttcgagatcctggaggctatcaccagcctcatcaacagcaacatcacgccttgcctgcctactcgagggaccatcacggcctccggcgacctcgtccctctgtcctacattgctggaatcctgacgggtcggccgaacgcgaaggccgtcactgcagaaggcgagatcgtggacgcttccgaggcattccgcctcgccggcatcccagatgggttcttcgagttgcagcccaaagaaggcctcgccctcgtcaacggaacagccgtcggctcgggccttgcctccattgtcctcttcgatgccaacattctgacggtcatggcggttgttctctccgctctgttctgcgaggtgatgcaggggaagccagagtataccgaccacctcacccacaagctgaagcaccatcctggacagatcgaggcggcggcgatcatggagcacatacttgaaggtagctcatacatgaagatggcgaagaaactccacgagatggatccactgcagaaaccaaagcaggacagatacgctctccgcacttcgccccaatggctcggtcctcagattgaggtcatccgagctgcaaacaagtccatcgagagggagatcaactccgttaacgataaccccctcatcgacgtctcacggaacaaagctctccatggaggcaacttccaagggacacccattggcgtctccatggacaacaccaggctcgccgttgccgccattgggaagctcatgtttgcccagttctccgagctcgtcaacgatttctacaacaatggcctgccttcgaatctctccggagggcgaaacccgagccttgactacggtttcaagggcgcagagatcgccatggcttcctactgctccgagctgcaagccctcgccaatccggtgaccaaccacgtccagagcgccgagcagcacaaccaggatgttaactctcttgggctgatctctgcaagaaagacagccgaagccgtcgaaatactgaagcttatgaccaccactttcttggttgggctctgccaagccgtggatttgagacatctggaggaaaatttgaagtttgctgtgaagaacaccgtgagtcaggtcgccaagcgaatcctcaccatgggggttaatggcgagctccacccctcgaggttctgcgagaaggaccttctcaacgtgatcgagagggagtatgttttcgcctatgccgacgacccatgcaactccgtctaccctctgatgccaaagctgaggcaggtactggtagagcacgcaatcaacaacggcgagaaggaaaaggaccccaacacttccatcttccagaaaataaccgccttcgaagaggagctcaaaatggcactgcccaaggaggtggaggccgtaagagttgccttcgagatgggctcctcggcgatcgaaaacagaatcagggactgcaggtcctatcctctgtacttattcgtgaaagaagtgggcgccggcttcctctctggcgacaagacaatctccccgggagaagagttcgacaaggtcttcatcgccatctcaaaggggaaggcgatcgatcccctgctcaaatgcttgcaggactggaacggagctcctcttcccatctgttag

SEQ ID NO: 3 - protein, 751 aa, Vanilla planifolia
  MISC_FEATURE (1)..(751): Vpl03Ag07441.1 allele gene product
  MISC_FEATURE (1)..(751): Vanilla planifolia ′Daphna′
MVRISPQLTRTAARYINPIPSSQVLQVGKPKLNFLSSESSRQLLCIKMENVLDMEGLCQKGSDPLGWGAAAAVVGGSHLDEVKRMVQEFRQPVVSLRGAELKISQVAAVAAGAASAVELAEAARAGVKASSDWVAESMNAGGDHYGVTTGFGSTSHRRTKMGGALQKELIRFLNAGIFGSGKDNTLPPPAVRAAMLVRINTLLQGYSGIRFEILEAITSLLNSKVSPCLPLRGTITASGDLVPLSYIAGILTGRPNARAITADGTTVDAAEAFRLAGISNGFFDLQPKEGLALVNGTAVGSGLASMVLFEANILAVMAEVLSAVFCEVMQGKPEYTDHLTHKLKHHPGQIEAAAIMEHVLEGSSYMKMAKKLHEMDPLQKPKQDRYALRTSPQWLGPQIEVIRAATKSIEREINSVNDNPLIDVSRNKALHGGNFQGTPIGVSMDNTRLAIAAIGKLMFAQISELVNDNYNNGLPSNLSGGRNPSLDYGFKGAEIAMAAYCSELQYLANPVTNHVQSAEQHNQDVNSLGLISSRKTAEAVEILKLMTSTFLMALCQAIDLRHLEENLKNSVKSAVSLAARRILTMGSNGELHPSRFCEKDLIKVIEREFVFAYADDPCSSTYPLMQKLRQVLVEHALGNGDKEKSPNSSIFHKIADFEEELKATLPKEVEATRVAFEKGLSAIPNRINECRSFPLYRFVREELGAGFLTGEKAKSPGEEFDKVFNAISEGKIIDPLLECLKEWNGAPLPIS

SEQ ID NO: 4 - DNA, 2256 bp, Vanilla planifolia
  misc_feature (1)..(2256): Vpl03Ag07441.1 allele
  misc_feature (1)..(2256): Vanilla planifolia ′Daphna′
atggtcaggatatctcctcaactaacccggactgcagctcgctatataaaccccatcccctcttcccaagttctccaagtcgggaagccaaagcttaatttcctgtcttcagaatcttcgcgacagcttctctgcattaaaatggagaatgtgcttgacatggaaggtttgtgccagaaggggtcggaccctttgggttggggagcagcagcggcagtggttggggggagccatttggacgaggtgaagcggatggtgcaggaatttcgccagccggtggtgagtctccgaggggccgagctaaagatatcgcaggtcgcggccgtggccgctggagcagcctccgccgttgaactcgcggaggcagcacgtgccggcgtcaaggccagcagcgattgggtggcagaaagcatgaatgcgggtggtgaccactacggcgtaaccactgggtttggctccacctcccaccgccgcaccaagatgggaggcgcccttcagaaagagctcatcagattcctgaatgccggcatcttcggctccgggaaagataacaccctgccgccaccggctgtccgagcagccatgcttgtgaggatcaacacactgcttcagggctactccggcatccggttcgagatcctcgaagccatcacaagtctcctcaacagcaaggtctctccttgcctccccttaagaggcaccatcaccgcctccggagacctcgtcccgctctcttatatcgctggaatcttgactggccgccctaacgccagggccatcactgctgatggcaccactgtcgacgccgcagaggctttccgcctggccggaatctcaaacgggttcttcgacctgcagcccaaggaaggtctagctctagtcaacggtaccgccgttgggtccgggctggcttccatggttctgtttgaggcaaacatccttgcggtcatggcggaggttctctcagctgtgttctgcgaggtgatgcaggggaagccggagtacaccgaccacctcactcacaaactgaagcaccatcccggccaaatcgaggctgccgccatcatggagcacgtgctcgaaggcagctcttacatgaaaatggcgaagaagctccacgagatggatcctttgcagaagccgaaacaggatcgctatgctctccgcacgtcgcctcagtggctgggccctcagattgaagtaatccgcgccgcaaccaagtcgatagagagggagataaattctgtgaacgacaaccctttgattgacgtttcaagaaacaaggctctccatggaggcaacttccagggaacaccaatcggcgtctccatggacaatactaggctcgccattgcagcgattgggaagctcatgttcgctcaaatctcagagctggtcaacgacaactataacaatggcctgccgtcgaatttatccggcggacgaaacccgagcttggattacggtttcaaaggcgcggaaattgccatggccgcctactgctccgagcttcagtaccttgccaatccggtgactaaccatgtgcaaagcgcagagcagcacaaccaggacgtgaactcgttgggactgatctcttcgaggaagacggctgaagcagtcgagattctaaagctcatgacttccaccttcttgatggcgctctgccaagccattgacctccggcatctggaggagaacctgaagaactctgtaaagagcgcggttagcctggcggcgagaaggatactaaccatggggtctaatggcgagctgcacccctcgaggttctgcgaaaaagacttaattaaggtgatcgagagggagttcgtgttcgcctatgccgacgatccctgcagctccacctatcccttaatgcagaagctaaggcaggtcctcgtcgagcatgctctcggcaatggagataaggagaagagtcccaactcctccatcttccataaaattgctgattttgaggaggagctgaaggccacgctaccaaaggaggtggaagcaacaagggtcgctttcgagaaaggcttatcggcgataccgaacaggatcaacgaatgcaggtcttttcctctttacaggtttgtcagggaggagctgggcgcggggttcctcaccggggagaaggccaagtcgccgggggaggagtttgacaaggtattcaacgccatcagcgagggcaagatcatcgatcccctcctcgagtgtttgaaggagtggaatggagctcctctccccattagctag

SEQ ID NO: 5 - protein, 708 aa, Vanilla planifolia
  MISC_FEATURE (1)..(708): Vpl03Ag07445.1 allele gene product
  MISC_FEATURE (1)..(708): Vanilla planifolia ′Daphna′
MEHARENVLEMEGLCQKGWDPLGWGEAAAAVEGSHFDEVKRMVQEFRQPVVSLRGAELKISQVAAVAAGAASAVELADEARAGVKASSDWVAESMSAGGDHYGVTTGFGSTSHRRTKLGGALQKELIRFLNAGIFGSGKDNTLPPPAVRAAMLVRINTLLQGYSGIRFEILEAITSLLNSKVSPCLPLRGTITASGDLVPLSYIAGILTGRPNARAITADGTTVDAAEAFRLAGISNGFFDLQPKEGLALVNGTAVGSGLASMVLFEANILAVMAEVLSAVFCEVMQGKPEYTDHLTHKLKHHPGQIEAAAIMEHVLEGSSYMKMAKKLHEMDPLQKPKQDRYALRTSPQWLGPQIEVIRAATKSIEREINSVNDNPLIDVSRNKALHGGNFQGTPIGVSMDNTRLAIAAIGKLMFAQISELVNDNYNNGLPSNLSGGRNPSLDYGFKGAEIAMAAYCSELQYLANPVTNHVQSAEQHNQDVNSLGLISSRKTAEAVEILKLMTSTFLVALCQAIDLRHLEENLKNSVKSAVILAAKRILTVGSNGELHPSRFCEKDLIKVIEREYVFAYADDPCSSTYPLMQKLRQVLVEHALRNGEKEKSPNSSIFHKIAAFEEELKATLPKEVEATRVAFEKGLSVVPNRINECRSFPLYRFVREELAAGFLTGEKARSPGEEFDKVFNAISEGKVIDPLLECLKEWNGAPLPIS

SEQ ID NO: 6 - DNA, 2127 bp, Vanilla planifolia
  misc_feature (1)..(2127): Vpl03Ag07445.1 allele
  misc_feature (1)..(2127): Vanilla planifolia ′Daphna′
atggagcacgcgagggagaatgtgcttgaaatggaaggtttgtgccagaaaggatgggatcctctgggttggggagaagcggcggcggcggtagagggaagccatttcgacgaggtgaagcggatggtgcaggagttccgccagccggtggtgagtctcagaggggccgagctaaagatatcgcaggtcgccgccgtggccgccggagcagcctccgccgttgagctagcggatgaagcacgtgccggcgtcaaggccagcagcgattgggtggcagagagcatgagtgcgggtggcgaccactacggcgtaaccactggctttggctccacctcccaccgccgcaccaagttgggaggcgcccttcagaaagagctcatcagattcttgaacgccggcatcttcggctcggggaaagataacacgctaccgccaccggctgtccgagcagccatgctcgtgaggatcaacacactgcttcagggctactccggcatccggttcgagatcctcgaagccatcacaagtctcctcaacagcaaggtctctccttgcctccccctaagaggcaccatcaccgcctccggcgacctcgtcccgctctcttatatcgctggaatcttgactggccgccctaacgccagggccatcacagctgatggcaccactgtcgacgccgcagaggctttccgcctcgccggaatctcaaatgggttcttcgacctgcagcccaaggaaggtctagctctagtcaacggtaccgccgttgggtccgggctggcttccatggttctgtttgaggcaaacatccttgcggtcatggcggaagttctctctgctgtgttctgcgaggtgatgcaggggaagccggagtacaccgaccacctcactcacaagctgaagcaccatcccggccaaatcgaggccgccgccatcatggagcacgtgctcgaaggcagctcttacatgaaaatggcgaagaagctccacgaaatggatcctctgcagaagccgaaacaggatcgctatgctctccgcacgtcgcctcagtggctgggccctcagattgaagtaatccgcgccgcaaccaagtcgatagagagggagataaattctgtgaacgacaaccctttgattgacgtttcaagaaacaaggctctccatggaggcaacttccagggaacacccatcggcgtctccatggacaacactaggctcgccattgccgcgattgggaagctcatgtttgctcaaatctcggagctggtcaacgacaactataacaacggcctgccgtcgaatctatccggcggacgaaacccgagcttggattacggtttcaaaggcgcggagattgccatggccgcttactgctccgagcttcagtaccttgccaatccggtgactaaccatgtgcaaagcgcggagcagcacaaccaggacgtgaactcgttgggattgatctcttccagaaagacggctgaggcggtagagattctaaagctcatgacttccaccttcttggtggcgctatgtcaagccattgacctcaggcatctggaggagaacctgaagaactctgtaaagagcgcggttatccttgcggccaagaggatactaaccgtgggatctaatggcgagctgcacccctcgaggttctgcgaaaaggacttgattaaggtgatcgagagggagtacgtgttcgcctatgccgacgatccctgtagctccacctatcctttgatgcagaagctaaggcaggtactcgtcgagcatgctctccgcaatggcgagaaggagaagagccccaactcctccatcttccacaaaattgcagcttttgaggaggagctgaaggccacgctaccaaaggaggtggaagcaacaagggtcgctttcgagaaaggcttatcggtggtaccgaacaggatcaacgaatgcaggtcttttcctctttacaggtttgtgagggaggagctggccgcggggttcctcaccggggagaaggccaggtcgccgggggaggagttcgacaaggtattcaacgccatcagtgagggcaaggtcatcgatcccctcctcgagtgtttgaaggagtggaatggagctcctctccccattagctag

SEQ ID NO: 7 - protein, 701 aa, Vanilla planifolia
  MISC_FEATURE (1)..(701): Vpl03Bg07223.1 allele gene product
  MISC_FEATURE (1)..(701): Vanilla planifolia ′Daphna′
MCLTWKVCARRVGPFGLGSSSGSGWGSHLDEVKRMVQEFRQPVVSLRGAELKISQVAAVAAGAASAVELAEAARAGVKASSDWVAESMNAGGDHYGVTTGFGSTSHRRTKMGGALQKELIRFLNAGIFGSGKDNTLPPPAVRAAMLVRINTLLQGYSGIRFEILEAITSLLNSKVSPCLPLRGTITASGDLVPLSYIAGILTGRPNARAITADGTTVDAAEAFRLAGISNGFFDLQPKEGLALVNGTAVGSGLASMVLFEANILAVMAEVLSAVFCEVMQGKPEYTDHLTHKLKHHPGQIEAAAIMEHVLEGSSYMKMAKKLHEMDPLQKPKQDRYALRTSPQWLGPQIEVIRAATKSIEREINSVNDNPLIDVSRNKALHGGNFQGTPIGVSMDNTRLAIAAIGKLMFAQISELVNDNYNNGLPSNLSGGRNPSLDYGFKGAEIAMAAYCSELQYLANPVTNHVQSAEQHNQDVNSLGLISSRKTAEAVEILKLMTSTFLMALCQAIDLRHLEENLKNSVKSAVSLAARRILTMGSNGELHPSRFCEKDLIKVIEREYVFAYADDPCSSTYPLMQKLRQVLVEHALGNGDKEKSPNSSIFHKIADFEEELKATLPKEVEATRVAFEKGLSAIPNRINECRSFPLYRFVREELGAGFLTGEKAKSPGEEFDKLFNAISEGKIIDPLLECLKEWNGAPLPIS

SEQ ID NO: 8 - DNA, 2106 bp, Vanilla planifolia
  misc_feature (1)..(2106): Vpl03Bg07223.1 allele
  misc_feature (1)..(2106): Vanilla planifolia ′Daphna′
atgtgcttgacatggaaggtttgtgccagaagggtcggaccctttgggttggggagcagcagcggcagtggttgggggagccatttggacgaggtgaagcggatggtgcaggaatttcgccagccggtggtgagtctccgaggggccgagctaaagatatcgcaggtcgcggccgtggccgctggagcagcctccgccgttgaactcgcggaggcagcacgtgccggcgtcaaggccagcagcgattgggtggcagaaagcatgaatgcgggtggtgaccactacggcgtaaccactgggtttggctccacctcccaccgccgcaccaagatgggaggcgcccttcagaaagagctcatcagattcctgaatgccggcatcttcggctccgggaaagataacaccctgccgccaccggctgtccgagcagccatgcttgtgaggatcaacacactgcttcagggctactccggcatccggttcgagatcctcgaagccatcactagtctcctcaacagcaaggtctctccttgcctccccctaagaggcaccatcaccgcctccggagacctcgtcccgctctcttatatcgctggaatcttgactggccgccctaacgccagggccatcactgctgatggcaccactgtcgacgccgcagaggctttccgcctcgccggaatctcaaacgggttcttcgacctgcagcccaaggaaggtctagctctagtcaacggtaccgccgttgggtccgggttggcttccatggttctgtttgaggcaaatatccttgcggtcatggcggaggttctctcagctgtgttctgcgaggtgatgcaggggaagccggagtacaccgaccacctcactcacaaactgaagcaccatcccggccaaatcgaggccgccgccatcatggagcacgtgctcgaaggcagctcttacatgaaaatggcgaagaagctccacgagatggatcctttgcagaagccgaaacaggatcgctatgctctccgcacgtcgcctcagtggctgggccctcagattgaagtaatccgcgccgcaaccaagtcgatagagagggagataaattctgtgaacgacaaccctttgattgacgtttcaagaaacaaggctctccatggaggcaacttccagggaacaccaattggcgtttccatggacaatactaggcttgccattgcagcgattgggaagctcatgttcgctcaaatctcagagctggtcaacgacaactataacaatggcctgccatcgaatttatccggcggacgaaacccgagcttggattacggtttcaaaggcgcggaaattgccatggccgcctactgctccgagcttcagtaccttgccaatccggtgactaaccatgtgcaaagcgcagagcagcacaaccaggacgtgaactcgttgggactgatctcttccaggaagacggctgaagcagtcgagattctaaagctcatgacttccaccttcttgatggcgctctgccaagccattgacctccggcatctggaggagaacctgaagaactctgtaaagagcgcggttagccttgcggcgagaaggatactaaccatggggtctaatggcgagctgcacccctcgaggttctgcgaaaaagacttaattaaggtgatcgagagggagtacgtgttcgcctatgccgacgatccctgcagctccacctatcccttaatgcagaagctaaggcaggtcctcgtcgagcatgctctcggcaatggtgataaggagaagagtcccaactcctccatcttccataaaattgctgattttgaggaggagctgaaggccacgctaccaaaggaggtggaagcaacaagggtcgctttcgagaaaggcttatcggcgataccgaacaggatcaacgaatgcaggtcatttcctctttacaggtttgtaagggaggagctgggcgcggggttcctcaccggggagaaggccaagtcgccgggggaggagtttgacaagttattcaacgccatcagcgagggcaagatcatcgatcccctcctcgagtgtttgaaggagtggaatggagctcctctccccattagctag

SEQ ID NO: 9 - protein, 356 aa, Vanilla planifolia
  MISC_FEATURE (1)..(356): Vpl_s027Bg25947 allele gene product
  MISC_FEATURE (1)..(356): Vanilla planifolia ′Daphna′
MAAKLLFFLLFLVSALSVALAGFEEDNPIRSVTQRPDSIEPAILGVLGSCRHAFHFARFARRYGKSYGSEEEIKKRFGIFVENLAFIRSTNRKDLSYTLGINQFADLTWEEFRTNRLGAAQNCSATAKGNHRFVDGVLPVTRDWREQGIVSPVKDQGSCGSCWTFSTTGALEAAYTQLTGKSISLSEQQLVDCASAFNNFGCNGGLPSQAFEYVKYNGGIDTEQTYPYLGVNGICNFKQENVGVKVIDSINITLGAEDELKHAVGLVRPVSVAFEVVKGFNLYKKGVYSSDTCGRDPMDVNHAVLAVGYGVEDGIPYWLIKNSWGTNWGDNGYFKMELGKNMCGVATCASYPIVAV

SEQ ID NO: 10 - DNA, 1071 bp, Vanilla planifolia
  misc_feature (1)..(1071): Vpl_s027Bg25947 allele
  misc_feature (1)..(1071): Vanilla planifolia ′Daphna′
atggcagctaagctcctcttcttcctactcttcctggtctccgccctctccgtcgcgctcgccggtttcgaagaagacaatccaatccggtccgttacacaaaggcctgactcgatcgagcctgccatcctcggcgtccttggcagctgccgccacgccttccacttcgcacggttcgcccgcaggtacgggaagagctacggatcggaggaggagatcaagaagaggttcgggatcttcgtggagaatctagcgtttatccggtccactaatcggaaggatctgtcgtataccctaggaatcaaccaattcgccgacttgacctgggaggaattccggaccaatcgccttggtgcggcgcagaactgctcggcgactgcgaagggaaaccaccggtttgtcgatggcgtgcttcctgtaacgagggattggagggagcaagggatagtgagccctgtaaaggaccaaggaagctgtggatcttgctggactttcagtactactggagcactagaggctgcatatacacagctaactggaaagagcatatcattatctgaacagcaacttgtggactgtgcctcagcattcaataactttggatgcaatggaggtttgccttcccaagcctttgaatacgttaagtacaatggaggcatcgacacagaacagacttatccataccttggtgtcaatggtatctgcaacttcaagcaggagaatgttggtgtcaaggtcattgattcgataaacatcaccctgggtgctgaggatgagttgaagcatgcagtgggcttggtgcgtccagttagcgttgcatttgaggttgtgaaaggtttcaatctgtacaagaaaggtgtatacagcagtgacacctgtggaagagatccaatggatgtgaaccacgcagttcttgccgtcggttatggagtcgaggacgggattccttattggctcatcaagaactcatggggtacaaattggggtgacaatggctactttaagatggagctcggcaagaacatgtgtggtgttgcaacttgcgcatcttatcccattgtggctgtgtag

SEQ ID NO: 11 - protein, 356 aa, Vanilla planifolia
  MISC_FEATURE (1)..(356): Vpl_s027Bg25938 allele gene product
  MISC_FEATURE (1)..(356): Vanilla planifolia ′Daphna′
MAAKLLFFLLFLVSALSVALAGFEEDNPIRSVTQRPDSIEPAILGVLGSCRHAFHFARFARRYGKSYGSEEEIKKRFGIFVENLAFIRSTNRKDLSYTLGINQFADLTWEEFRTNRLGAAQNCSATAHGNHRFVDGVLPVTRDWREQGIVSPVKDQGSCGSCWTFSTTGALEAAYTQLTGKSTSLSEQQLVDCASAFNNFGCNGGLPSQAFEYVKYNGGIDTEQTYPYLGVNGICNFKQENVGVKVIDSINITLGAEDELKHAVGLVRPVSVAFEVVKGFNLYKKGVYSSDTCGRDPMDVNHAVLAVGYGVEDGIPYWLIKNSWGTNWGDNGYFKMELGKNMCGVATCASYPIVAV

SEQ ID NO: 12 - DNA, 1071 bp, Vanilla planifolia
  misc_feature (1)..(1071): Vpl_s027Bg25938 allele
  misc_feature (1)..(1071): Vanilla planifolia ′Daphna′
atggcagctaagctcctcttcttcctactcttcctggtctccgccctctccgtcgcgctcgccggtttcgaagaagacaatccaatccggtccgttacacaaaggcctgactcgattgagcctgccatcctcggcgtccttggcagttgccgccacgccttccacttcgcacggttcgcccgcaggtacgggaagagctacggatcggaggaggagatcaagaagaggttcgggatcttcgtggagaatctagcgtttatccggtccactaatcggaaggatctgtcgtataccctaggaatcaaccaattcgccgacctgacctgggaggaattccggaccaatcgccttggtgcggcgcagaactgctcggcgactgcgcatggaaaccaccggtttgtcgatggcgtgcttcctgtaacgagggattggagggagcaagggatagtgagccctgtaaaggaccaaggaagctgtggatcttgctggactttcagtactactggagcactagaggctgcatatacacagctaactggaaagagcacatcattatctgaacagcaacttgtggactgtgcctcagcattcaataactttggatgcaatggaggtttgccttcccaagcctttgaatacgttaagtacaatggaggcatcgacacagaacagacttatccataccttggtgtcaatggtatctgcaacttcaagcaggagaatgttggtgtcaaggtcattgattcgataaacatcaccctgggtgctgaggatgagttgaagcatgcagtgggcttggtgcgtccagttagcgttgcatttgaggttgtgaaaggtttcaatctgtacaagaaaggtgtatacagcagtgacacctgtggaagagatccaatggatgtgaaccacgcagttcttgccgtcggttatggagtcgaggacgggattccttattggctcatcaagaactcatggggtacaaattggggtgacaatggctactttaagatggaactcggcaagaacatgtgtggtgttgcaacttgcgcatcttatcccattgtggctgtgtag

SEQ ID NO: 13 - protein, 235 aa, Vanilla planifolia
  MISC_FEATURE (1)..(235): Vpl_s027Ag26221.1 allele gene product
  MISC_FEATURE (1)..(235): Vanilla planifolia ′Daphna′
MQWAWCVQLALHLREQGIVRPVKDQGSCGSCWTFSTTGALEAAYTQLTGKSTSLSEQQLVDCASAFNNFGCNGGLPSQAFEYVKYNGGIDTEQTYPYLGVNGICNFKQENVGVKVIDSINITLGAEDELKHAVGLVRPVSVAFEVVKGFNLYKKGVYSSDTCGRDPMDVNHAVLAVGYGVEDGIPYWLIKNSWGTNWGDNGYFKMELGKNMCGVATCILSHCGCVEAIIANNVSK

SEQ ID NO: 14 - DNA, 708 bp, Vanilla planifolia
  misc_feature (1)..(708): Vpl_s027Ag26221.1 allele
  misc_feature (1)..(708): Vanilla planifolia ′Daphna′
atgcagtgggcttggtgcgtccagttagcgttgcatttgagggagcaagggatagtgaggcctgtaaaggaccaaggaagctgtggatcttgctggactttcagtactactggagcactagaggctgcatatacacagctaactggaaagagcacatcattatctgaacagcaacttgtggactgtgcctcagcattcaataactttggatgcaatggaggtttgccttcccaagcctttgaatacgttaagtacaatggaggcatcgacacagaacagacttatccataccttggtgtcaatggtatctgcaacttcaagcaggagaatgttggtgtcaaggtcattgattcgataaacatcaccctgggtgctgaggatgagttgaagcatgcagtgggcttggtgcgtccagttagcgttgcatttgaggttgtgaaaggtttcaatctgtacaagaaaggtgtatacagcagtgacacctgtggaagagatccaatggatgtgaaccacgcagttcttgccgtcggttatggagtcgaggacgggattccttattggctcatcaagaactcatggggtacaaattggggtgacaatggctactttaagatggagctcggcaagaacatgtgtggtgttgcaacttgcatcttatcccattgtggctgtgtagaagctatcattgctaataatgtgtcaaaatag

SEQ ID NO: 15 - protein, 233 aa, Vanilla planifolia
  MISC_FEATURE (1)..(233): Vpl06Ag12707.1 allele gene product
  MISC_FEATURE (1)..(233): Vanilla planifolia ′Daphna′
MGRGKIEIKRIENTTNRQVTFCKRRNGLLKKAYELSVLCDAEVALIIFSSRGRLYEYSNSSTKSTIERYKKACANSANSFIEINSPQYFQQEAAKLRHQIQILQSANRNLMGDSLSSLSIKELKQLESRLERGISRIRSKKHEMLFAELEFMQKREAELQNENMYLRMKITENERAQANIANAALEALPTFDARNYYPINMFEPEDNYDHHQDQTALHLGYEGKGEQDARDVL

SEQ ID NO: 16 - DNA, 702 bp, Vanilla planifolia
  misc_feature (1)..(702): Vpl06Ag12707.1 allele
  misc_feature (1)..(702): Vanilla planifolia ′Daphna′
atggggagagggaagattgagataaagaggatcgagaacactacaaaccgtcaggtgactttctgcaagaggaggaacgggttgctgaagaaggcctacgaactctcagtactctgcgatgcagaggtcgccctgatcatcttttccagccgcggccgtctctatgaatactccaacagcagcacaaagtcaacaattgaaaggtacaagaaagcatgtgctaacagtgccaattcattcatcgagataaactctccgcaatatttccagcaggaagcagcaaagttgcgtcatcagatacagattttacaaagtgcaaatagaaaccttatgggcgattcgttaagttcactttccataaaggagctgaagcaactagaaagcagactagaacgaggaatctcaaggattagatcgaagaagcatgagatgctgtttgcggaacttgagttcatgcagaaaagggaagcagagctacaaaatgaaaatatgtacctcagaatgaagataacagagaacgagagagcacaagctaacatagcaaatgcagcacttgaagctcttcctacatttgatgcaagaaattattaccccatcaatatgttcgaaccggaggacaactacgatcaccaccaagaccagaccgcacttcatcttggttatgaaggcaaaggcgaacaagatgctcgagatgtgctgtaa

SEQ ID NO: 17 - protein, 216 aa, Vanilla planifolia
  MISC_FEATURE (1)..(216): Vpl09Ag19274.1 allele gene product
  MISC_FEATURE (1)..(216): Vanilla planifolia ′Daphna′
MNPNPTSPLTSTAEQQLEWMEMVMEMENLADLNDQMTLPQPPLLVSNPFSPSPIKTNTTSQPAFCTTSANAMPSADYFTTTRSWPAAADYHPPPGSVVAMRDMIFRMAAMQPVYIDPESVKAPRRRNVRISSDPQSVAARHRRERISEKIRILQHLVPGGTKMDTASMLDEAINYVKFLKLQVRSLERAASSAGSACFCFPVPLDYEYFVGVEHKV

SEQ ID NO: 18 - DNA, 651 bp, Vanilla planifolia
  misc_feature (1)..(651): Vpl09Ag19274.1 allele
  misc_feature (1)..(651): Vanilla planifolia ′Daphna′
atgaaccccaaccccacctctccattaacgtcgacggcggaacagcagttggaatggatggaaatggtaatggagatggagaacctcgctgatctcaatgaccagatgaccctgcctcagcctcctctgctggttagcaacccattctcgccatcacccatcaagaccaacacaacgtcgcagccagctttctgcaccacgtcggcaaacgcgatgccgagtgccgattattttaccaccacccggtcatggccagccgctgcggattaccaccctccgccgggctcggtggtggcgatgcgggacatgatatttcgcatggcggcgatgcagcccgtctacatcgaccctgaatcggtgaaggcgccaaggcggagaaacgtgaggatatcgagtgatccgcagagcgtggcggcgcgtcaccggagggagaggatctccgagaagatacgcatcctacagcatctagtccccggtgggaccaagatggacacggcgtccatgctcgacgaggccattaactacgtcaaatttctcaagttacaggtgcggtcgctcgagcgggcggccagcagcgcggggagcgcgtgtttctgcttccccgtgccgttggattatgagtacttcgttggagttgagcacaaagtttga

SEQ ID NO: 19 - protein, 495 aa, Vanilla planifolia
  MISC_FEATURE (1)..(495): Vpl01Ag01494.1 allele gene product
  MISC_FEATURE (1)..(495): Vanilla planifolia ′Daphna′
MSSTAQGYGRGGEEEQVLLHPPQPNLAGFICHVPQQSRREKLRFPPEESPAGATTPSIPAIYQEADNYYLPVPSFPCSSSSSMTSHDVLLGGEGVYQIQKLGFSLSLSPAVGSLGPFTGYAAVLNRSRFVEPARQLLEEICVLSRGVALGGRDLPDDLMVDGDVQEYVRDKEQQWKKTKLISMLDEVYRRYRFYYRQVQTVTTSFEAVAGLNSAAPYVFTALKSMSRHFRSLKNAIGSHLNRAAGKEDLGKEGFSGLGLINAETYSQRGNSSATFRQPPIWRPQRGLPERAVTVLRAWLFEHFLHPYPTDTDKQMLAKKTGLTRNQVSNWFINARVRLWKPMVEEVHNLDTRQSQKAVAAETSDKHALQPSSSAATSSSNPPPAPQNSSVQTNQNPYTKSSNNQLQHIAHHIQEPYNFVFDGFCSHAQTGGIPLGGTSGVSLTLGLHQHDGVGVPQPSGFGLEECNDAYVFGAMDGQDRHMGKDIGAHLLHDFVG

SEQ ID NO: 20 - DNA, 1488 bp, Vanilla planifolia
  misc_feature (1)..(1488): Vpl01Ag01494.1 allele
  misc_feature (1)..(1488): Vanilla planifolia ′Daphna′
atgtcttcgacggcacaagggtatggtagaggaggcgaggaggaacaagtccttcttcacccgccgcagcctaacctggctggtttcatctgccacgtaccgcagcagagccggagggagaagctccggttcccgccagaggaatctccggcgggagccaccacgccgtcgattcccgcgatttatcaagaagcggataactactacctcccggttccctccttcccatgctcctcctcctcgtcgatgacctcgcacgacgttctgcttggcggggaaggcgtctaccagatccagaagcttggcttctccctctcgctctcgccggcggtcggttctcttggaccgtttaccgggtacgctgccgtactgaaccggtccaggttcgtggaaccggctcggcagctgctagaggagatatgcgtgcttagtcgaggagtggccttaggtggacgcgatctgccggatgatctgatggtggatggggatgtgcaggaatatgtgcgtgataaggagcagcaatggaagaagacgaagcttatttccatgttggatgaggtctatagaaggtataggttttactaccggcaagtgcagactgtgaccacatcatttgaagcagtagctggcttaaactctgcggctccttatgtttttactgctctgaagtccatgtccaggcacttcagatctctaaagaatgcaataggaagtcacctcaacagggctgcaggaaaggaagaccttggtaaagagggcttttctgggttaggtctcatcaatgctgaaacatactcgcaaagaggaaacagctctgctactttcaggcagcccccgatttggcggcctcaaagagggcttcctgaacgcgcagtgaccgttctccgggcctggctatttgaacatttcttgcatccgtaccctacggatacagacaagcaaatgttggccaagaaaactggcctgacaagaaaccaggtttctaactggtttataaatgcaagggtaaggctgtggaagccaatggtggaggaagtacacaacctcgatacgagacaatcacaaaaggctgtggctgctgaaaccagtgacaagcatgctctgcagccatcgtcttctgctgcaacatcttccagtaatccgccacccgcaccccaaaacagcagcgttcaaacaaaccaaaacccatatacgaaatcatcaaacaatcagctccagcacattgctcaccacatccaagagccgtataactttgtttttgatgggttctgcagccacgctcagactggtggtattccccttgggggaacctccggcgtttctctaactcttggtctgcaccaacacgatggagtgggcgtgccgcagccgagcggcttcggcctggaagaatgcaacgacgcctatgtgtttggggcgatggatgggcaggacaggcacatggggaaggacattggcgcgcatctgttgcacgactttgttggttga

SEQ ID NO: 21 - protein, 459 aa, Vanilla planifolia
  MISC_FEATURE (1)..(459): Vpl06Ag13482.1 allele gene product
  MISC_FEATURE (1)..(459): Vanilla planifolia ′Daphna′
MSHVAANLSYLEPRIFLKEGEGEMDVVSLLTTILLLLIPIGADDMGCDVELAVWRSNGAGRIPVNVETFGAVGDGIADDTQAFVSAWKKACSLNDAVFLVPEQRRYKVNATRFKGPCQDKLIIQLSGTIVAPEEPDHWDPKNPRVWLVFSNLQGIRIQGGGVIDGSGSKWWASSCKLNKTNPCRGAPTALTIDSSSKVRVKGLTIQSAQQMHFTIFRSKTIRVSGVRVKSPPDSPNTDGIHISESSDVSIHNCKIGTGDDCISIVNASSNIVMKQIECGPGHGISIGSLGKDNTMAVVKGVVLDTATLQGTTNGLRIKTWQGGSGYVQSVRFENVKMEDVQNPIIIDQFYCDSPKACMNQTSAVKISQVIYRNIHGTSKTPKAMKFSCSDTVPCSNIVLNNINLETENGTAETFCNCAMGFDYGYVKPAADCLHYDGTSHCNGGSLVNTIEHRHLHTEL

SEQ ID NO: 22 - DNA, 1380 bp, Vanilla planifolia
  misc_feature (1)..(1380): Vpl06Ag13482.1 allele
  misc_feature (1)..(1380): Vanilla planifolia ′Daphna′
atgagccatgttgcagcaaatcttagttatttggagcccagaatttttctgaaagaaggagagggagaaatggatgtagtttcattgctgaccaccattctgcttttgctgatcccaattggagcagatgacatgggttgtgatgtcgaactggctgtgtggagaagcaatggggctggaagaataccagtgaacgtcgaaacctttggtgcagttggagatggcattgcggatgatacgcaggcttttgttagcgcatggaagaaggcgtgctctttgaacgatgctgtcttcttggttccagaacaacgccgttacaaagtcaatgccacaagattcaaaggaccatgccaagacaaactaatcatccaattaagtggaacaatcgtagccccagaagagcctgaccattgggatccaaaaaatccacgggtttggttagttttctcaaacctacagggcatcagaatacagggtggtggagtcattgatggttcaggcagcaaatggtgggcttcatcttgcaagctcaacaaaaccaatccatgcagaggagcaccaacggcattaaccatcgactccagctccaaggtaagagttaaaggccttaccatccagagtgctcaacagatgcatttcactatcttcagatcaaaaacaataagagtctcaggtgttcgagtaaagtcccctccagatagtcctaacacagatggtatacacatcagtgagtcctctgatgtgagcatccataattgcaagattggaacgggagatgattgtatttcaatcgtgaatgcaagctcaaacatcgtgatgaagcagattgagtgtggaccaggccatggaataagcattgggagcctggggaaagataacactatggcagtagttaaaggggttgttctagacactgcaacactccaaggaactacaaatggcttacgaatcaaaacttggcaaggaggatcaggttatgttcaatccgttcgatttgaaaatgtaaagatggaggatgtccaaaatccaatcattattgatcagttttattgtgactcgccaaaagcctgtatgaaccagacatcagcagtaaagataagtcaagtcatatatagaaacattcacggcacctcaaaaactccaaaggcaatgaaattttcatgtagtgacacagtgccatgcagcaacatagtgttgaacaatattaacttggaaacagaaaacggaacagcagagaccttctgtaactgcgctatgggttttgactatggatatgttaagccagcagcagactgcctacactacgatggtacctcccattgcaatggtggttcgcttgttaacactatagagcacaggcacctgcatacagaactatga

SEQ ID NO: 23 - protein, 227 aa, Vanilla planifolia
  MISC_FEATURE (1)..(227): Vpl07Ag14471.1 allele gene product
  MISC_FEATURE (1)..(227): Vanilla planifolia ′Daphna′
MSSSSASSPTFSLDHLSSTPYEQLCYVHCNFCDTVLAVSVPCSSFFKTVTVRCGHCTNLFSVNMRGLLLQSAEQLHLGHPFLSPLQHNLLDELACPSSSLVMDHPTAMANNAASQLAKATEEELPNPIPTVLNRPPEKRQRVPSAYNRFIKDEIQRIKAGNPDITHREAFSAAAKNWAHFPHIHFGLMADQTMKKSSVRQQEGGDVLLKDGFYAATAANMNMGIAPF

SEQ ID NO: 24 - DNA, 684 bp, Vanilla planifolia
  misc_feature (1)..(684): Vpl07Ag14471.1 allele
  misc_feature (1)..(684): Vanilla planifolia ′Daphna′
atgtcgtcctcctcagcatcatcacctaccttttctttggaccacctctcctccactccatatgagcaactctgttacgtccactgcaacttttgcgatactgtcctcgccgttagtgtcccctgttcgagcttcttcaagacggtgacagtgaggtgtggtcactgcaccaatctcttctccgtcaacatgagagggcttctccttcagtcggctgagcagctgcacctcggccatcctttcctttctccacttcagcataatctcttggatgagctggcttgcccgtcttcgagcttagtgatggaccatcctactgccatggccaacaacgccgcatcgcagctggccaaggcaacggaagaagaacttcctaatccgatacccacagttcttaatagaccaccggagaagaggcagagagtgccatcggcttacaaccgcttcatcaaagatgaaatccagcggatcaaagcaggcaaccccgatatcacccacagagaagccttcagtgccgcagcaaagaactgggctcacttcccacacatccatttcggcctcatggctgatcaaaccatgaagaagagcagcgtgcgacagcaggagggtggcgacgttcttctcaaggatggattctacgctgccacggcagccaatatgaacatgggaatagctcctttctaa

SEQ ID NO: 25 - protein, 48 aa, Vanilla planifolia
  MISC_FEATURE (1)..(48): MADS-box domain from Vpl04Ag08481.1 allele gene product
  MISC_FEATURE (1)..(48): Vanilla planifolia ′Daphna′
RIENKINRQVTFSKRRNGLLKKAYELSVLCDAEVALIIFSSRGKLYEF

SEQ ID NO: 26 - protein, 232 aa, Vanilla planifolia
  MISC_FEATURE (1)..(232): Vpl04Ag09199.1 allele gene product
  MISC_FEATURE (1)..(232): Vanilla planifolia ′Daphna′
MGRGKIEIKRIENTTNRQVTFCKRRNGLLKKAYELSVLCDAEIALIVFSSRGRLYEYSNNSIKATIEKYKKACADNANPGSIVEINSQQYYHQESAKLRHQIQLLQNANRHLMGDGLSSLTLKELKQLENRLERGITRIRSRKHELLFAEIEYMQKREVELQNDNMYLRAKIADNERAQQETIVQVATDFEALQTFDSRNYYHVNMLEAAPHYSHHQDQTALHLGYETKGDT

SEQ ID NO: 27 - DNA, 699 bp, Vanilla planifolia
  misc_feature (1)..(699): Vpl04Ag09199.1 allele
  misc_feature (1)..(699): Vanilla planifolia ′Daphna′
atggggagaggaaagattgagatcaagaggatcgagaacactacgaatcggcaagtgaccttctgcaagaggaggaacggtcttctgaagaaagcatatgagctttcagtcctctgtgacgctgaaatcgcactgattgttttctccagccgagggcgcctctatgagtactccaacaacagcattaaagcaacaattgagaagtataagaaggcttgtgcagacaatgcaaatcctggatccattgtggagatcaattctcagcagtactatcatcaagaatcagcaaaattgcgccatcaaattcagctattgcaaaatgcaaacaggcatttaatgggggatggactaagttcccttactttaaaagagctcaaacagcttgaaaaccgacttgaaagaggcatcacaaggattagatcaaggaagcacgagctactctttgcagaaattgagtacatgcagaaaagagaagtggaacttcaaaacgacaatatgtatcttagagccaagattgcagataatgaacgagcacaacaagagactattgtacaagtagcaactgattttgaggcccttcaaacttttgactctagaaattactaccatgtcaacatgttggaagcagcacctcattattcacaccatcaagatcaaacagctctccatcttggatatgaaacaaaaggtgacacgtaa

SEQ ID NO: 28 - protein, 233 aa, Vanilla planifolia; identical to SEQ ID NO: 15
  MISC_FEATURE (1)..(233): Vpl06Ag12707.1 allele MADS-box protein
  MISC_FEATURE (1)..(233): Vanilla planifolia ′Daphna′

SEQ ID NO: 29 - DNA, 702 bp, Vanilla planifolia; identical to SEQ ID NO: 16
  misc_feature (1)..(702): Vpl06Ag12707.1 MADS-box allele
  misc_feature (1)..(702): Vanilla planifolia ′Daphna′

SEQ ID NO: 30 - protein, 233 aa, Vanilla planifolia
  MISC_FEATURE (1)..(233): Vpl06Ag12680.1 allele gene product
  MISC_FEATURE (1)..(233): Vanilla planifolia ′Daphna′
MGRGKIEIKRIENTTNRQVTFCKRRNGLLKKAYELSVLCDAEVALIIFSSRGRLYEYSNSSTKSTIERYKKACANSANSFIEINSPQYFQQEAAKLRHQIQILQSANRNLMGDSLSSLSIKELKQLESRLERGISRIRSKKHEMLFAEIEFMQKREAELQNENMYLRMKITENERAQANIANAALEALPTFDARNYYPINMFEAEDNYDHNQDQTALHLGYEGKGEQDARDVL

SEQ ID NO: 31 - DNA, 702 bp, Vanilla planifolia
  misc_feature (1)..(702): Vpl06Ag12680.1 allele
  misc_feature (1)..(702): Vanilla planifolia ′Daphna′
atggggagagggaagattgagataaagaggatcgagaacactacaaaccgtcaggtgactttctgcaagaggaggaacgggttgctgaagaaggcctacgaactctcagtactctgcgatgcagaggtcgccctgatcatcttttccagccgcggccgcctctatgaatactccaacagcagcacaaagtcaacaattgaaaggtacaagaaagcatgtgctaacagtgccaattcattcatcgagataaactctccgcaatatttccagcaggaagcagcaaagttgcgtcatcagatacagattttacaaagtgcaaatagaaaccttatgggcgattctttaagttcactttccataaaggagctgaagcaactagaaagcagactagaacgaggaatctcaaggattagatcgaagaagcatgagatgctgtttgcggaaattgagttcatgcagaaaagggaagcagagctacaaaatgaaaatatgtacctcagaatgaagataacagagaacgagagagcacaagctaacatagcaaatgcagcacttgaagctcttcctacgtttgatgcaagaaattattaccccatcaatatgttcgaagcggaggacaattacgatcacaaccaagaccagaccgcacttcatcttggttatgaaggcaaaggcgaacaagatgctcgagatgtgctgtaa

SEQ ID NO: 32 - protein, 159 aa, Vanilla planifolia
  MISC_FEATURE (1)..(159): Vpl10Ag20060.1 allele gene product
  MISC_FEATURE (1)..(159): Vanilla planifolia ′Daphna′
MPQSICIFCMWKKTARKEDEKKAKWCLGLSSYFLLSTWAPQATVKRGQKVNLGNHKLATSQAGNTESATLEPKEKMGRGKIEIKRIENTTNRQVTFCKRRNGLLKKAYELSVLCDAEVALIIFSNRGRLYEYANNSKDHETDIFRAIFFIFLPKPYDKI

SEQ ID NO: 33 - DNA, 480 bp, Vanilla planifolia
  misc_feature (1)..(480): Vpl10Ag20060.1 allele
  misc_feature (1)..(480): Vanilla planifolia ′Daphna′
atgcctcaatcaatatgtatattctgcatgtggaagaagacagcaagaaaggaagacgaaaagaaagccaaatggtgtcttggactcagctcctacttccttctcagcacatgggctcctcaagcaacagtgaaaagagggcaaaaggtcaatctcggcaaccacaagctggctacatctcaagcagggaacacggagtctgcaaccctggagcccaaggagaagatgggtagggggaagatagagatcaaaaggattgagaacacaacaaaccggcaagttaccttctgtaaacgccgcaacggtcttctcaagaaggcctatgagctctccgtcctctgtgacgccgaggtcgccctcatcatcttctccaaccgcggccgcctctacgaatacgccaacaacagtaaggatcacgagactgacatcttccgtgccatcttcttcatttttcttcccaaaccctacgacaaaatctga

SEQ ID NO: 34 - protein, 154 aa, Vanilla planifolia
  MISC_FEATURE (1)..(154): Vpl01Ag00567.1 allele gene product
  MISC_FEATURE (1)..(154): Vanilla planifolia ′Daphna′
MTRHWAKFSGDTRRRLHPRNHKLLSKNIFDNFSLGKLKTTPKAKGGKLKDNGALHGFLPETQRSGWSPRRRSAGKKEIKRIENTTNRQVTFCKRRNGLLKKAYELSVLCDAEVALIIFSSRGRLYEYANNSFSEGSSSELQAMMDLANSEEREP

SEQ ID NO: 35 - DNA, 465 bp, Vanilla planifolia
  misc_feature (1)..(465): Vpl01Ag00567.1 allele
  misc_feature (1)..(465): Vanilla planifolia ′Daphna′
atgacacgacattgggccaaattttctggtgacacaagaaggcgactccatccaagaaaccacaagttattgagcaagaacatcttcgacaatttctccttgggaaaactgaagacaacacccaaggcaaagggaggaaaattgaaggacaatggagccctgcatgggttccttccggagacgcagagaagtggatggagcccaaggagaagatcggcagggaaaaaagagattaagaggatagagaacacgacgaacaggcaagttaccttctgcaagcgtcggaacggtctccttaagaaggcttacgagctatccgtcctatgcgacgccgaggttgccctcatcatcttctccagccgcggccgtctctatgaatacgccaacaacagcttctctgaaggatcaagctctgaacttcaagcgatgatggatctagctaacagtgaggaacgggagccttag

SEQ ID NO: 36 - protein, 907 aa, Vanilla planifolia
  MISC_FEATURE (1)..(907): Vpl02Ag05172.1 allele gene product
  MISC_FEATURE (1)..(907): Vanilla planifolia ′Daphna′
MCVNLQIDPVRAIIELFTALSEPLRYLRNPKPRLALLTDAMSQIKSKREDYNDKIHTAELAGQTSTHQIMDWLNKVAAVDSKVAKIQTDFLQQRSCRWCINRRVAKSLATIKDLIDAASKIDHVATKLAPKGVQEVSDVPPASVNKTTYLSQLQVYIQDDECRIIGIWGMGGVGKTTLLQSLNNELLPSAEADHSPQQFDHVIWAVVSKDYSQGNLRRDIARRLGLHLSDDRDAQAMEIFDFLKNKSFLLLLDDLWQPVDFAAIGIPQPLHYGATNRKVVFTTRLEGVCGQMQTQRKIKLECLNVEDAWRLFQEKVSEETLRAHARIPQLARRVAKECDGLPLALVVIGTTMSTKKTPKEWQNAITLLQKSRLYEIHGLEDKILPKLKFSYDNLANDIIRECFLLCSLWPEDFSISKTDLIECWMGHGLVGVGKLDNINEAYDSGHALIGGLKAACLLESGDDEEREVKMHDVVRDLALWIASDCGEKHNRFIVQTGEVGAEYAQSEVERISLVRSHLSDTSGLPTNCSKLETLMLQESISFKSILTGFCDGTLALTYLDLSYTAIYDLPSEIGRLGNLRYLNVSHTNIPSLPWQIGCLSELRFLLLRKLDSIVIPSGVIKKLVKLSVIDMTDTWCESWLELAALRGCLKGVGLVLEAVEDLKQLARLSNVPTWRLGIRKLVGLVEPLQLLSPLQLGSRSIRSSIQSLKIEFCESLQLVTMDGDGGIGSSSSWCLPRLEEVELRSLPELKDIVWRGVIPADSLPSLTVLKISACSKLTNISWVLRLPSLEELTVHRCGEMQQVVDEDQGGDAEENVVTNEAFPSLKKLCLSELQSLRMFSGGSLAFPSLEVLQVLRCPEMKKLPFGAEMAGKRLKEIWGDRAWWDALQMENEARAALQPRFKRREPR

SEQ ID NO: 37 - DNA, 2724 bp, Vanilla planifolia
  misc_feature (1)..(2724): Vpl02Ag05172.1 allele
  misc_feature (1)..(2724): Vanilla planifolia ′Daphna′
atgtgtgtcaacttacagattgatcccgtccgcgccatcatcgaattgtttacagccttgtcggaaccactgcgatatctcagaaaccccaaacctagacttgctctccttaccgacgccatgagccagatcaaaagcaagagggaggactacaacgacaagatccatacggccgagctcgctggacaaactagtacccaccagataatggactggttgaacaaggtcgccgccgtggattccaaggtcgccaaaatccagactgacttcctccagcagcgttcttgtcgatggtgcatcaaccggcgagtggcgaagagcctcgccaccataaaggatttgatcgacgcagccagcaaaatcgaccacgtggccacgaagttggctccgaaaggcgtgcaggaggtgtccgacgtcccgcccgcttcggtgaacaagacgacttacctgtcccaactccaggtctacatccaagacgacgaatgccggatcatcggaatatggggcatgggaggtgtaggaaagacgacgcttttacaaagcctcaataacgagctgctgccctctgcagaagccgaccatagtccacaacaattcgatcacgtcatctgggctgtagtttccaaggactacagccaaggaaatctgcggcgtgatatcgcgcggcggctgggtcttcacttgagcgacgatagagacgcacaagccatggagatattcgacttcttgaagaacaagagcttccttctcctcctcgacgacctgtggcagcccgtggactttgccgcgataggcatccctcaacctctccactatggggccaccaaccgtaaggtggtgttcaccactcggctagagggcgtctgtgggcagatgcagacacagcggaagatcaagttggagtgccttaacgtggaggatgcatggcggctctttcaggagaaggtcagcgaggagaccctccgtgcccacgccagaatcccacagcttgctcggcgtgtcgcaaaggagtgtgacggcctcccgctcgccctcgtggtcatcggtaccaccatgagcacgaagaagactcctaaggagtggcagaacgccatcaccctgctgcagaaatcccggctgtacgagatccacggcctggaggacaagatcctgcccaagctcaaattcagctatgacaatcttgccaacgacatcatccgcgaatgtttcctcctctgctcgctgtggcctgaggacttttccatctctaagaccgatctcatcgaatgctggatgggtcacgggcttgtgggagtgggcaaactggacaacatcaacgaagcgtacgacagtggacacgcgctcatcggaggactcaaggctgcttgcctccttgagtccggagacgacgaggaaagggaggtcaagatgcacgatgttgttcgcgacctcgcactctggatcgcctccgattgcggcgagaagcacaataggttcatcgttcaaactggtgaggttggcgctgaatatgctcagagcgaggtggagaggatttctcttgtccgcagccatctaagtgacacctccggtttgccgaccaattgttccaagctcgagactctgatgctccaagaaagtatctccttcaagtctatcttgacaggcttttgtgatggaacattggccttgacgtaccttgatctctcttataccgccatctacgacctgccctctgaaatcggccgtttgggtaatctccggtacctcaacgtctcccacaccaacatcccatctcttccatggcagatcggttgtctcagtgagctgaggtttctcttgttgcggaagctagattctatcgttatacctagcggggtaattaagaaactcgtgaagctctctgtgatcgacatgaccgatacttggtgcgagagctggctggaactcgctgcgctgcggggttgtttgaaaggcgtgggactcgtcctggaggcggttgaagaccttaagcagcttgcccggctctcaaatgtgccgacttggcgcttgggtataaggaagctagtgggattggtggagcctctgcaattgctttcaccattacagctaggtagccgcagcattagatcgagcatccagtcacttaaaatcgagttctgcgagagcttgcagttggtgacaatggacggtgatggtggcatcggcagcagctcaagctggtgtcttccgaggctggaggaagtcgagctcaggagcctcccggagttgaaggacattgtctggagaggagttattcctgcggactcgttgccgagccttacagtgctgaagatttccgcttgcagcaaattgacgaatatctcatgggtgctccgactcccgagcctagaggagctcacggtgcaccgttgcggcgagatgcagcaagttgtcgatgaggaccaaggcggtgatgcagaggaaaatgttgtgaccaatgaggcatttcccagccttaagaagctctgtttgagcgaattgcaaagtctgagaatgttcagcggtggatctctagcgtttccatcgcttgaggtgttgcaggtactgaggtgcccggagatgaagaagcttccttttggggcggaaatggccggcaaaaggctgaaggaaatttggggggatagggcgtggtgggatgcgttgcagatggagaacgaagcaagggccgctcttcagccgcgtttcaagagaagggagccacggtag

SEQ ID NO: 38 - protein, 1059 aa, Vanilla planifolia
  MISC_FEATURE (1)..(1059): Vpl14Ag25867.1 allele gene product
  MISC_FEATURE (1)..(1059): Vanilla planifolia ′Daphna′
MAMILDAFVGICLEKLAVIVEEKATTVMGVKDELQKLQRRLGKIHTALEDAERRRLENAAINLWVLELKDILYDADDIIDLCLVKSPHLLSDGGDRLSSFSPTVCCHFLKFLCCFTSLSDRYAIGKGIQNLNERLKEIYEEKGIFDCVPKKPIIEKTLVNIGDTSPMLKFVVGRAIKEATKKLVEEMSFGFNEKKSYRVFAITGMSGIGKTTLAQRIFNNDKMKSYFQIKSWVCVSPTYSEIELLKQIIRDASGVYGDAKTKSELLPILGSALAGKSLFLVLDDVWRADVWIELLQNPIQMAGIEARILITTRDVNIAKLMRSFYIHPVTQLPDETGWEMLCRILFQEEQTEDMHRLREIGIKIVKKCHGLPLAIKAIAGVLSKREPNNKEWEEVLSRDAWSNSNLPEELRGPLYLSYDDLPSPLKQCFLYCSLWTEDWIIGREDLIRMWIAEGFIKVNGGDSTLMEDTGMDYYNELIRRNLLQLDPRFVDGSICSMHDMLRSLAEFLCEGERPRNFHGRNDKSSTKVRRMIISRPSELPNGQGDPTLQNRYLRTLICLDGAQFKGTSSLGRLECLRVLLINDVEIGSLPHNIGNLIHLRFLDLARTDIKELPKSIELLVNLQFLILSGCTALSALPQNITKLYNLRCLNLDDTPLIDFIPKGIGMLRQLNDLSGFVVGDDNIANPPEKQHSSLDEMNSLIKLRVLHVRKIELAQPRAVNLEKFSHLRELMLSCSPCSSQKTDEQIKQIEALFDEFKPTERLELLHISNYFGTQYPRWLMSSSLVTRFTYLTIVNLEGNESCLQLPPLGQLPNLKSLEITNAFAIKVIGQDFLVGGAGTSSHANKNTAFPKLELLKIKNLRDMEEWCASEEFKNGNESQQLLLWPCLNTLYLDNCNKLRSLPVGLKHAPLTMLKIRHAYSIKQVCHLSSLAGQLTLHCNRSLERVSDLPDLKYLIIEGSPNLKHVENLGSLQKIELHEDSMEFFLDWLPGLVQKKAAVDNDDNGLFVRLFCKEEKFRCCMQGGSYWSNMKQISRFEVTLLGKNGYIKYSKNPFSYNTNF

SEQ ID NO: 39 - DNA, 3180 bp, Vanilla planifolia
  misc_feature (1)..(3180): Vpl14Ag25867.1 allele
  misc_feature (1)..(3180): Vanilla planifolia ′Daphna′
atggccatgatcttggatgcattcgtcggcatatgcctcgagaagctcgccgttatcgtcgaggaaaaggcgaccacggtaatgggagtgaaagatgagctccaaaagcttcagagaaggctggggaagatccacacggcgctcgaggatgccgagaggaggagactcgaaaacgccgccatcaacctctgggtcctggagctcaaggacatactttacgacgccgacgacattattgacctctgtttggttaaatcccctcatttgctgtcggatggcggcgatcggctctcgtccttttctcctacggtatgctgccattttctcaaatttctctgctgctttacttctctctctgatcgctatgcgattggtaagggaatacaaaacctaaacgagagattgaaagagatttatgaagaaaagggaatctttgattgtgtacctaagaaacctattatagagaaaacgctggttaacatcggtgatacatctcccatgttgaagtttgtcgtggggagagccattaaggaagctactaagaaactggtggaggagatgtcttttgggtttaatgaaaagaaatcctaccgtgtctttgccattacagggatgagtgggattgggaagaccacgttggctcaaaggatttttaacaatgataaaatgaagtcatatttccaaataaaatcatgggtctgcgtttctccaacatattctgaaatcgagttgctgaagcaaatcataagagatgcttcaggggtctacggagatgctaaaacaaagtcggagctcctaccaattcttggatctgccttagctggaaagagtctatttcttgttcttgacgatgtttggagggcagatgtgtggattgagttgcttcaaaatccaatacaaatggctgggattgaagcgaggatcttgatcacaaccagagatgttaatattgctaagctaatgaggagtttttatattcatccagtaacgcagcttccagatgaaaccggttgggaaatgctttgcaggatactttttcaggaggagcaaacagaggacatgcaccgcttgagagagattgggatcaaaattgttaaaaaatgccatggtcttcctcttgccatcaaagccattgctggggttctatcaaagagggaacctaacaacaaagagtgggaagaagtccttagtcgtgatgcctggtccaactccaaccttcccgaagagcttagaggtccattataccttagctatgacgatcttccatctcctctcaagcagtgcttcctttattgctccttgtggactgaggattggattattggtcgtgaagaccttattcgaatgtggattgcggaaggctttataaaggtgaatggaggtgattcaacattgatggaagacacgggaatggattactacaatgagttgataagaagaaatcttctgcagctagatcctcgctttgttgatggctctatatgtagtatgcatgatatgttgcgatctcttgcagaatttctttgtgaaggggaaagacctagaaatttccatggccgaaatgataaatcatcaacgaaagttcgccgcatgatcatttccagaccaagtgaattaccaaatggacagggtgatccaacattgcaaaacagatatttaaggacactaatttgcttagatggcgctcaatttaaaggtactagttcacttggaagacttgaatgccttcgagttcttctcattaatgatgtggagatcggaagccttcctcataatataggtaatctaatccacctaaggtttttggatttggctcgtaccgacataaaggagctcccaaaatccattgagcttttagtaaatctacaattcttgatactctcaggatgtacagctttgtcagctctcccacaaaacattaccaagttatacaatcttagatgtctaaatcttgatgacacgccacttattgattttatcccaaaaggcatagggatgttacgacaacttaatgacctcagtggttttgttgtaggggatgacaacattgctaatccaccagagaaacaacatagtagtttggacgagatgaactctcttatcaaacttagagtcttgcatgtacgcaaaatagaacttgcacaaccaagagctgtgaatcttgaaaagttttcacatctgagagagttgatgttgtcttgtagcccttgcagctcccaaaaaactgatgaacaaatcaagcagattgaagcactctttgatgaatttaaacccactgaacgtttagaattacttcatatctcaaactattttggtacacaatacccaaggtggttgatgtcttcatccttagtgacaagatttacttacttgacgattgttaatttagagggaaatgaatcatgtcttcaacttccaccactgggacaattacccaatttgaaatctcttgagataactaatgcttttgctattaaagtcataggccaagatttccttgttggtggagcaggcacttcttctcatgcaaacaaaaacacagctttccctaagcttgagctgcttaagatcaagaacttgcgagacatggaagaatggtgtgcaagtgaagagtttaaaaatggaaatgagtcccaacaacttttgttgtggccttgtctcaacacactctacttggataattgcaacaaattgagatcacttccagttggtttaaagcatgctcccttgactatgttgaagattagacatgcttatagtatcaaacaagtttgccacctttcctccctcgctggtcagcttaccctccattgcaatcgtagtttggagagagtctctgatctacccgatcttaagtacttgataatcgaaggttctcctaatttgaaacatgttgagaatttgggttcattgcaaaagatagaactccatgaggattccatggaattcttcttagattggttaccaggtcttgtccaaaagaaagcagcagttgataatgatgataatggtctttttgttcgcctattctgcaaagaagagaagtttagatgttgtatgcaaggaggctcttattggtccaacatgaagcagatttctagatttgaagttaccctattagggaaaaatggatatattaagtactccaagaatccttttagttacaacaccaacttttga

SEQ ID NO: 40 - protein, 1055 aa, Vanilla planifolia
  MISC_FEATURE (1)..(1055): Vpl_s056Ag26537.1 allele gene product
  MISC_FEATURE (1)..(1055): Vanilla planifolia ′Daphna′
MAMVLDAFVGICVDKMTGLITEKVVMVLGVKDDLKRLQRRMTRIHKFLEAAERRRLDDPTLDHWVAELKDVAYDADDIIDLCRVHGARLLNDHRPSPPLRRLACFNFLFSCLSSVPHRYQIGESIRTLNDKLEEIDKDRVLLNLEESSIRGQQIVTTVNIRQTSSMAEFDVVGEEIEEATNKLVKLIVDEQGNTCRVSAITGMGGIGKTTLAQKVFNNKKIMAEFQVRIWVCVSQTYNEIDVLQQVIRAAGGHHGEARIKSELQPILKSAVFGKSVFLVLDDVWRADVWLSLLRNPLRSAEAMVRVLVTTRDENVAREMGAVHIHRVQQLSNDASWAMLCRKVFNEGQQEEISNVFDIGLLIVARCCGLPLAIKAIAGVLSKKGSNRKEWEKVLNNEAWSMSRLPEELRGALYLSFDDLPSPLKQCFVYCSLFPEDESLSRSDLVRLWVAEGFVKEQGDAFIEDTAKEYYGELIRRNLLQPDPMHYDESECIMHDLMRSLAKFLSQDETFTGNVTNTAASPAKLRRLAVENQETISTVASLVVEQKCLRTLLAFHGTRLFDNSDQLAGLTRLRVLRINSVDINRLPNSIGDLIHLRYLDLDDTDIEELPESIGSLTNLQFLNLRACEFLTKLPDNIMQLHNIRRLGLYNTPLRFIPKGIKNLGKLNDLSGFVVADPGEQSKVGYSSLGELGSLHQLRMLSIAKLERAQKGDLSLANLSHLVDLKLFYSKVDALTVKTEDISRVENVLEELCPPHCLEELCIRKYFGHRYPTWMQSPSFRAHFPFLTTLELSKNKFCTQLPPAGELPQLKLLFIKRADAVKRIGPEFLGIGTHPAIRTAFPKLEILKLYRILHLEEWTFGETEEAQVLPNLVLLPRLSSLEIEDCPMLASLPKGLEQSSMKRLYIAGALSLKAVSNLPNLTELVLIGNASLETISHLPALQVLAVISCPAFKLVEKVDALQRLNLVDHFMEAIPEWLVKFLRERKAGEGEGEVSADDFLLEMLCTEKALWGCLKGGSDWPIVETIPSFIAYDNVGKGFMRYTRSPFYYYTNVIMAN

SEQ ID NO: 41 - DNA, 3168 bp, Vanilla planifolia
  misc_feature (1)..(3168): Vpl_s056Ag26537.1 allele
  misc_feature (1)..(3168): Vanilla planifolia ′Daphna′
atggcgatggtcctcgatgcattcgtgggcatctgcgtggataagatgacgggattgatcaccgagaaggtggtcatggttctgggagtcaaggacgacctcaaaaggcttcagagaaggatgacgaggatacacaagttcctggaggccgccgagaggcggaggctcgatgacccaactctcgatcactgggtcgccgagctcaaggacgtcgcctacgacgccgatgacataatcgacctttgccgagtgcacggtgctcgcctcctcaacgatcaccgaccgtccccacccttgagaagactggcatgctttaacttcctgttttcctgcttatccagcgttccacatcgctaccaaatcggtgaaagcattagaactcttaacgacaaactagaggagatcgataaagacagggtgctgctcaacttagaagagagctcaatacgaggccagcaaattgtgaccacggtgaacatacgccaaacttcctctatggcggagttcgacgtggtgggagaagagatcgaagaggctacgaacaagttagtgaagctgatagttgatgaacagggtaatacatgtcgcgtatctgccatcacggggatgggaggaatagggaagaccaccttagctcaaaaagtgttcaataacaagaagatcatggcggaattccaggtaaggatttgggtttgcgtttcgcagacgtataacgaaatagacgtgctgcaacaagtcattagagcggccggaggtcaccacggcgaggctcgaatcaagtccgagctgcaaccaattcttaagtcggccgtgtttgggaagagcgttttcctcgtcttggatgacgtttggagggcagatgtgtggttgagtttgcttcgtaatccgcttcgaagcgcagaagcaatggtaagggtcttggtcacaacgagagacgagaatgttgcgagggagatgggggctgtgcacatacaccgcgtgcagcagttatcgaacgatgcgagttgggcgatgctctgtcgtaaggtgttcaacgagggacagcaggaggagataagcaacgtgttcgatatcggccttctgatagttgcaaggtgttgcggtcttcctcttgcgatcaaagccattgcaggtgttttatccaagaagggatcaaatagaaaagaatgggagaaggttctcaacaatgaggcatggtcaatgagcaggcttcccgaagagcttaggggtgccttgtaccttagctttgacgaccttccctctccactcaagcaatgcttcgtgtactgctccctctttccagaagacgaatcgcttagccgcagtgacctcgttcggttatgggtcgcagaaggcttcgtgaaggagcaaggagatgcctttatagaagacacggcaaaggaatactacggtgagttgatacgaaggaaccttctgcagcccgatccgatgcattacgatgaaagcgaatgcatcatgcatgatctgatgagatctcttgccaagtttttgtcccaggatgaaacctttactggaaacgtgacgaacacagctgcctcgcccgcaaagcttcgtcgtcttgctgtggaaaaccaagaaaccatatcaactgtagcgagtttggttgtcgagcagaagtgtttgagaactttgctcgcttttcatggcactcggttgtttgataacagtgatcagttggcagggcttacacgcctgcgtgtattgcgaatcaacagcgtggatatcaatagacttcccaactccataggtgatctgatacacttgcggtaccttgatctggatgacactgatatcgaagagttgcccgagtctattggatccctcactaaccttcagttcctcaacctccgtgcatgcgagttcttgacgaagctccccgataacatcatgcagctgcacaacataaggcgtctaggcctctacaatactccccttcgtttcataccaaaaggaatcaagaatctggggaagctcaacgatctcagtgggttcgttgtggctgatccaggggagcaatcaaaagttggctacagcagcttgggtgagcttggttctcttcatcagctcagaatgctcagcattgcaaagctcgagagggcgcagaaaggagacctatcccttgcaaacctaagtcatcttgtcgacttgaagctcttttactccaaagttgatgctcttacagtcaaaacagaggacatatcacgagttgagaatgtcttggaagagctttgtcctccacactgccttgaagaactctgtatcagaaaatactttgggcacagatatccaacgtggatgcaatctccatcttttcgagcacactttcccttcttaacaaccctggagctatcaaaaaacaaattttgcacacagctcccacctgcaggcgaattgcctcagttgaaactgcttttcattaaacgtgcagatgcagtaaaacgcataggacccgaattccttggcatcgggacgcatcccgccattagaacagccttccccaagctcgaaatcctcaaactttatcgtatccttcatctggaagagtggacctttggagaaacagaggaggcacaagttctccccaacctggttctgctacctcgcctgtcctctctggagattgaagactgccccatgcttgcatcgcttccaaagggcttggaacagtcttccatgaagcggttgtacatcgccggagctctcagtctgaaggcagtgagcaatctgcccaatttaacggagcttgtgctgatcggcaatgctagcttggaaaccatttctcatctccctgcgttgcaagtccttgctgttataagttgcccagcttttaagcttgtggagaaggtggacgctctgcaaagactgaatctcgttgaccatttcatggaagcgattccagaatggttggtgaagtttcttcgggagaggaaggcgggtgaaggtgaaggagaagttagtgcggatgacttcctgctggaaatgctgtgcaccgagaaagctctgtggggatgcctcaaaggaggttccgactggcccatcgttgaaaccattccatcctttatcgcgtatgacaacgtggggaaggggtttatgcgctacacgagaagccctttctactattatacaaacgtgatcatggcgaattga